SEQUENCE LISTING (2 sequences)

SEQ ID NO: 1 - DNA, 25 bp, Artificial sequence
  Synthetic oligonucleotide
gucucagccucuucucauuccugct

SEQ ID NO: 2 - DNA, 27 bp, Artificial sequence
  Synthetic oligonucleotide
agcaggaaugagaagaggcugagacau

(19) United States
(12) Patent Application Publication
(10) Pub. No.: US 2022/0023204 A1
(43) Pub. Date: Jan. 27, 2022

(54) BIOLOGICALLY ACTIVE DRY POWDER COMPOSITIONS AND METHOD OF THEIR MANUFACTURE AND USE

(71) Applicant: BOARD OF REGENTS, THE UNIVERSITY OF TEXAS SYSTEM, Austin, TX (US)

(72) Inventors: Hugh D.C. SMYTH, Austin, TX (US); Hairui ZHANG, Austin, TX (US); Zhengrong CUI, Austin, TX (US); Jieliang WANG, Austin, TX (US); Haiyue XU, Austin, TX (US); Yajie ZHANG, Austin, TX (US); Debadyuti GHOSH, Austin, TX (US); Jasmim LEAL, Austin, TX (US); Melissa SOTO, Austin, TX (US); Robert O. WILLIAMS, III, Austin, TX (US); Chaeho MOON, Austin, TX (US); Sawittree SAHAKIJPIJARN, Austin, TX (US)

(73) Assignee: BOARD OF REGENTS, THE UNIVERSITY OF TEXAS SYSTEM, Austin, TX (US)

(21) Appl. No.: 17/235,771

(22) Filed: Apr. 20, 2021

Related U.S. Application Data
(60) Provisional application No. 63/012,792, filed on Apr. 20, 2020.

(51) Int. Cl. A61K 9/00; A61K 47/46; A61K 45/06

(52) U.S. Cl. CPC A61K 9/0075; A61K 45/06; A61K 47/46

(57) ABSTRACT

Dry powder compositions comprising biologically active polynucleotide molecules and methods for the manufacture of such dry powders are provided. In some aspects, dry powders of the embodiment comprise expressible or regulatory (e.g., siRNA) polynucleotide molecules complexed with nanoparticles. Dry powders comprising viable virus and bacteria are also provided.

This application claims the benefit of priority to U.S. Provisional Application No. 63/012,792, filed on Apr. 20, 2020, the entire contents of which are hereby incorporated by reference.

### INCORPORATION OF SEQUENCE LISTING

The sequence listing that is contained in the file named “UTSBP1238US ST25.txt”, which is 1 KB (as measured in Microsoft Windows®) and was created on Apr. 20, 2021, is filed herewith by electronic submission and is incorporated by reference herein.

### BACKGROUND OF THE INVENTION

### 1. Field of the Invention

The present disclosure relates generally to the field of pharmaceutical formulation, biologics and the manufacture of the same. More particularly, it concerns dry powder compositions that include, viruses, bacteria and polynucleotide molecules and methods of preparing powder compositions, such as by thin-film freezing.

### 2. Description of Related Art

Recent drug development has employed to new treatment moieties, such as compositions that include biologically active polynucleotides. For example, mRNA is being studies for delivery of therapeutic proteins and antigens. Likewise, CRISPR technology is being explored for gene replacement and small interfering RNA (siRNA) is being developed for knock-down of undesirable gene activities. Additionally, whole cell (e.g., bacterial cell) and viral compositions offer potential new therapeutic and vaccination moieties. However, in all of these cases, new formulations and formulation methods are required that allow for the compositions to be stabilized and to maintain biological activity. Likewise, new formulations and methodologies are required to provide efficient ways to delivery therapies to patients in need.

### SUMMARY OF THE INVENTION

In some embodiments, the present disclosure provides dry powder compositions comprising biologically active polynucleotide molecules and at least a first excipient, said dry powder having been produced by an ultra-rapid freezing process (URF), wherein the polynucleotide molecules retain substantial biological activity and/or have been stabilized by the URF process. In some aspects, the polynucleotide molecules retain at least about 0.1%, 0.2%, 0.3%, 0.4%, 0.5%, 1%, 2%, 3%, 4%, 5%, 10%, 15%, 20%, 25%, 30%, 40% or 50% of a biological activity compared to an equal amount of the polynucleotide molecule in solution prior to the URF process. In some aspects, the polynucleotide molecules have been stabilized such that at least 50% more of the molecules in the powder are undegraded relative the same polynucleotide molecules in a solution. In some aspects, the URF process comprises thin film freezing (TFF). In some aspects, the polynucleotide molecules are double-stranded molecules. In some aspects, the polynucleotide molecules are single-stranded molecules or a mix of double-stranded and single-stranded molecules. In some aspects, the polynucleotide molecules comprise siRNA, shRNA, dsRNA, ssRNA, mRNA, plasmid DNA and/or DNA oligonucleotides.

In some aspects, the powder has a geometric particle size distribution Dv50, measured by dry Rodos method, of less than about 100 μm, 50 μm, 30 μm, 20 μm, 15 μm or 12 μm. In further aspects, the powder has a geometric particle size distribution Dv50, measured by dry Rodos method, of about 1 to 50 μm or 3 to 50 μm. In some aspects, the powder has a density of about 1.0 to g/cm³; 2.0 1.4 to 1.9 g/cm³; 1.4 to 1.9 g/cm³; or 1.5 to 1.7 g/cm³. In some aspects, the powder has a surface area of about 2.0 to 8.5 m²/g; 2.0 to 7.5 m²/g; 3.0 to 7.5 m²/g; 2.0 to 5.0 m²/g; 2.5 to 4.5 m²/g; or 3.0 to 4.0 m²/g. In some aspects, the first excipient comprises a sugar, or sugar alcohol. In further aspects, the sugar is a disaccharide. In some aspects, first excipient comprises lactose, trehalose, sucrose, mannitol or sorbitol. In some aspects, the first excipient comprises at least about 50% of the powder by weight. In further aspects, the first excipient comprises from about 50%, 55%, 60%, 65%, 70%, 75%, 80%, 85%, 90%, 95%, 99%, 99.1%, 99.2%, 99.3%, 99.4%, to about 99.5% of the powder by weight. In some aspects, the first excipient comprises a sugar, or sugar alcohol.

In some aspects, the dry powder compositions further comprise a pH buffering agent. In some aspects, the pH buffering agent comprises phosphate buffered saline (PBS), sodium acetate, or Mg²⁺ storage (SM) buffer. In some aspects, the pharmaceutical dry powder composition has a water content of less than 20%, 15% or 10%. In some aspects, the pharmaceutical dry powder composition has a water content of from about 0.5% to 10%, 1% to 10%, 1.5% to 8% or 2% to 5%. In some aspects, the dry powder compositions further comprise at least a second, third and/or fourth excipient. In further aspects, the second, third and/or fourth excipient comprises an amino acid or protein. In still further aspects, the second, third and/or fourth excipient comprises leucine or glycine. In some aspects, the second, third and/or fourth excipient comprises a polymer. In further aspects, the polymer comprises PEG, HPMC, PLGA, PVA, dextran, sodium alginate or PVP. In some aspects, the second, third and/or fourth comprises a sugar, or sugar alcohol. In further aspects, the powder comprises a mixture of two, three or more different sugars or sugar alcohols. In some aspects, the dry powder compositions further comprise a protein or a surfactant. In some aspects, the dry powder compositions further comprise casein, lactoferrin, Pluronic F68, Tyloxapol, or ammonium bicarbonate. In some aspects, the excipient comprises about 20%, 25%, 30%, 35%, 40%, 45%, 50%, 55%, 60%, 65%, 70%, 75%, 80%, 85%, 90%, 95%, 96%, 97%, 98%, 99%, 99.1%, 99.2%, 99.3%, 99.4%, 99.5%, 99.6%, 99.7%, 99.8%, to about 99.9% of the powder, such as from about 20% w/w to about 99.9% w/w of the powder.

In some aspects, the biologically active polynucleotide molecule comprises a virus or a virus-like particle (VLP). In further aspects, the virus is a non-enveloped virus. In still further aspects, the virus comprises an adeno-associated virus, adenovirus, an adeno-associated virus vector or an adenovirus vector. In some aspects, the virus comprises bacteriophage. In further aspects, the bacteriophage infects S. aureus and/or P. aeruginosa. In some aspects, the bacteriophage particles comprise phage PEV2 or T7 phage. In some aspects, the powder has a geometric particle size distribution Dv50, measured by dry Rodos method, of less than 15 μm. In some aspects, the powder has a geometric particle size distribution Dv50, measured by dry Rodos method, of less than about 20 μm, 15 μm or 12 μm. In some aspects, the powder has a geometric particle size distribution Dv50, measured by dry Rodos method, of about 3 to 15 μm, 4 to 12 μm or 5 to 10 μm. In some aspects, at least about 20%, 25%, 30%, 35%, 40%, 45%, to about 50%, of the particles have a size of 1-5 μm, such as about 20%. In some aspects, the first excipient comprises a sugar or sugar alcohol. In further aspects, the first excipient comprises lactose, trehalose, sucrose, mannitol or sorbitol. In some aspects, the dry power further comprises an amino acid. In further aspects, the amino acid comprises leucine or glycine. In some aspects, the dry powder compositions comprise sucrose and leucine. In further aspects, sucrose and leucine are present in a ratio of from about 50:50, 55:45, 60:40, 65:35, 70:30, 75:25, 80:20, 85:15, 90:10, to about 95:5, such as from about 50:50 to about 95:5, about 60:40, from about 70:30 to about 90:10; or from about 75:25 to about 80:20 (sucrose:leucine).

In some aspects, the biologically active polynucleotide molecules comprise polynucleotide molecules encapsulated in lipid nanoparticles (LNPs). In some aspects, the biologically active polynucleotide molecule comprises a mRNA. In further aspects, the mRNA encodes an antigen. In some aspects, the dry powder composition further comprises an adjuvant. In some aspects, the adjuvant comprises aluminum salts, such as alum. In some aspects, the LNPs comprise ionizable lipids, phospholipids, cholesterol, lecithin and/or poly-(ethylene) glycol (PEG)-lipid. In some aspects, the LNPs comprise cationic lipids; DOPE; DPPC; DSPC; DMPE-PEG; DMG-PEG; DSPE-PEG; Dlin-MC3-DMA; phospholipids; PEG-lipid and/or cholesterol. In some aspects, the LNPs have an average particle size of between about 25 nm and 1000 nm, 50 nm and 1000 nm; 50 nm and 600 nm, or 80 nm and 200 nm. In some aspects, the first excipient comprises a sugar or sugar alcohol. In further aspects, the first excipient comprises lactose, trehalose, sucrose, mannitol or sorbitol. In some aspects, the dry powder compositions comprise from about 10%, 20%, 30%, 40%, 50%, 60%, 70%, 80%, 90%, 95%, 96%, 97%, 98%, to about 99% lactose, trehalose, sucrose, mannitol or sorbitol, such as from about 10% to about 99% or from about 50% to about 99.5% lactose, trehalose, sucrose, mannitol or sorbitol. In some aspects, the dry powder compositions comprise from about 80% to about 99% or from about 90% to about 99% sucrose.

In some aspects, the biologically active polynucleotide molecule comprises siRNA. In some aspects, the LNPs comprise ionizable lipids, phospholipids, cholesterol, lecithin and/or poly-(ethylene) glycol (PEG)-lipid. In some aspects, the LNPs comprise lecithin, cholesterol and/or polyethylene glycol (2000)-hydrazone-stearic acid. In some aspects, the LNPs comprise cationic lipids. In some aspects, the LNPs have an average particle size of about 50 nm, about 75 nm, about 100 nm, about 125 nm, about 150 nm, about 175 nm, about 200 nm, about 225 nm, about 250 nm, about 275 nm, about 300 nm, about 325 nm, about 350 nm, about 375 nm, about 400 nm, about 425 nm, about 450 nm, about 475 nm, or about 500 nm, such as between about 50 nm and about 500 nm, about 75 nm and about 250 nm, about 80 nm and about 200 nm, about 90 nm and about 175 nm, or about 100 nm and about 150 nm. In some aspects, the powder has a geometric particle size distribution Dv50, measured by dry Rodos method, of less than 15 μm. In some aspects, the powder has a geometric particle size distribution Dv50, measured by dry Rodos method, of less than about 20 μm, 15 μm or 12 μm. In further aspects, the powder has a geometric particle size distribution Dv50, measured by dry Rodos method, of about 3 to 15 μm, 4 to 12 μm or 5 to 10 μm. In some aspects, the powder has a mass median aerodynamic diameter between about 2 μm and 7 μm, 3 μm and 7 μm, 3 μm and 5 μm or 3.5 μm and 4.5 μm. In some aspects, the powder has a fine particle fraction (FPF) value of between about 25% and 60%, 30% and 50%, or 35% and 40%. In some aspects, the powder has a deposition in stages 4-7 in a Next Generation Impactor (NGI) of at least 10%, 15% or 20%. In further aspects, the powder has a deposition in stages 4-7 in a Next Generation Impactor (NGI) of between about 10% and 25%; 15% and 25%; 10% and 20% or 15% and 22%. In some aspects, the siRNA is less than 30 nucleotides in length. In some aspects, the siRNA is targeted to a human gene or a pathogen gene. In some aspects, the siRNA is targeted to TNF-α.

In some aspects, the biologically active polynucleotide molecules comprise polynucleotide molecules complexed with chitosan. In further aspects, the chitosan is PEGylated. In some aspects, the biologically active polynucleotide molecules comprise DNA complexed with chitosan. In some aspects, the DNA molecules have been stabilized such that at least 50% more of the molecules in the powder are undegraded relative the same polynucleotide molecules in a solution. In some aspects, the DNA comprises plasmid DNA. In some aspects, the dry powder compositions comprise DNA encoding CRISPR/Cas9 elements complexed with chitosan. In some aspects, the dry powder compositions comprise DNA encoding a guide RNA complexed with chitosan. In some aspects, the chitosan complexes have an average size of about 100 nm to 2000 nm. In some aspects, the chitosan complexes have an average size of about 100 nm to 1000 nm; 150 nm to 800 nm or 200 nm to 800 nm. In some aspects, the first excipient comprises a sugar or sugar alcohol. In some aspects, the first excipient comprises lactose, trehalose, sucrose, mannitol or sorbitol. In some aspects, the dry powder compositions comprise about 5%, 10%, 20%, 30%, 40%, 50%, 60%, 70%, 80%, to about 90%, of a sugar or sugar alcohol, such as from about 5% to 90% of a sugar or sugar alcohol. In some aspects, the dry powder compositions comprise from about 10% to about 90%, from about 10% to about 70%, or from about 10% to about 50% of a trehalose, sucrose, and/or mannitol. In some aspects, the powder has a geometric particle size distribution Dv50, measured by dry Rodos method, of less than about 100 μm, 50 μm, 30 μm, 20 μm, 15 μm or 12 μm. In some aspects, the powder has a geometric particle size distribution Dv50, measured by dry Rodos method, of about 1 to 50 μm or 3 to 50 μm. In some aspects, the powder has a density of from about 1.0 to g/cm³ to about 2.0 g/cm³, from about 1.4 to about 1.9 g/cm³, from about 1.4 to 1.9 g/cm³, or from about 1.5 to about 1.7 g/cm³. In some aspects, the powder has a surface area of about 2.0 to 8.5 m²/g; 2.0 to 7.5 m²/g; 3.0 to 7.5 m²/g; 2.0 to 5.0 m²/g; 2.5 to 4.5 m²/g; or 3.0 to 4.0 m²/g.

In some aspects, the biologically active polynucleotide molecules comprise genomic material. In some aspects, the genomic material comprises bacterial, eukaryotic or archaeal genomic material. In some aspects, the powder comprises intact cells. In some aspects, the powder comprises living cells. In some aspects, the powder comprises intact bacterial, eukaryotic or archaeal cells. In some aspects, the powder comprises intact bacterial cells. In some aspects, the powder comprises living bacterial cells. In some aspects, the bacterial cells comprise gram negative bacteria. In some aspects, the bacterial cells comprise gram positive bacteria. In some aspects, the first excipient comprises a sugar or sugar alcohol. In some aspects, the first excipient comprises lactose, trehalose, sucrose, mannitol or sorbitol. In some aspects, the first excipient comprises sucrose. In some aspects, the powder is formulated for administration via inhalation. In some aspects, the powder is formulated for use with an inhaler.

In other embodiments, the present disclosure provides inhalers comprising a dry powder composition of the present disclosure. In some aspects, the inhaler is a fixed dose combination inhaler, a single dose dry powder inhaler, a multi-dose dry powder inhaler, multi-unit dose dry powder inhaler, a metered dose inhaler, or a pressurized metered dose inhaler. In some aspects, the inhaler is a capsule-based inhaler. In some aspects, the inhaler is a low resistance inhaler. In some aspects, the inhaler is a high resistance inhaler. In some aspects, the inhaler is used with a flow rate from about 10 L/min to about 150 L/min. In some aspects, the flow rate is from about 20 L/min to about 100 L/min.

In yet other embodiments, the present disclosure provides methods of producing dry powder pharmaceutical composition comprising: (a) admixing an encapsulated biologically active polynucleotide molecule and a first excipient in a solvent to form a precursor solution; (b) depositing the precursor solution onto a surface at a temperature suitable to cause the solvent to freeze; and (c) removing the solvent to obtain the powder pharmaceutical composition. In some aspects, the methods further comprise: (d) disaggregating the powder pharmaceutical composition to reduce particle size and/or homogenize particle size.

In some aspects, the precursor solution comprises water. In some aspects, the powder pharmaceutical composition has a water content of less than 20%, 15% or 10%. In some aspects, the powder pharmaceutical composition has a water content of about 0.5% to 10%, 1% to 10%, 1.5% to 8% or 2% to 5%. In some aspects, the temperature in step (b) is about −40° C. to −180° C. In some aspects, the temperature in step (b) is about −50° C. to −150° C., −50° C. to −125° C., −55° C. to −100° C. or −65° C. to −75° C. In some aspects, the precursor solution comprises a pH buffering agent. In some aspects, the precursor solution has a pH of about 6.0 to 8.0, 6.5 to 8.0, or 7.0 to 7.8. In some aspects, the precursor solution comprises about 0.1% to 30%, 0.1% to 20%, 0.5% to 10% or 0.5% to 5% of the first excipient. In some aspects, the first excipient comprises a sugar or sugar alcohol. In some aspects, the precursor solution comprises about 0.1% to 5%; 0.1% to 3% or 0.5% to 5% of a trehalose, sucrose and/or mannitol. In some aspects, the precursor solution has a solids content of about 0.1% to 50%. In some aspects, the precursor solution has a solids content of about 0.1% to 20%. In some aspects, the precursor solution has a solids content of at least about 0.25%. In some aspects, the precursor solution has a solids content of 0.25% to 10%; 0.5% to 10%; 1% to 5% or 2% to 5%.

In some aspects, the biologically active polynucleotide molecule comprises virus or bacteriophage. In some aspects, the virus is a non-enveloped virus. In some aspects, the biologically active polynucleotide molecule comprises bacteriophage. In some aspects, the precursor solution comprises about 1×10⁶ to 1×10¹²; 1×10⁶ to 1×10¹¹; 1×10⁷ to 1×10¹⁰; or 5×10⁸ to 1×10⁹ plaque forming units/ml (PFU/mL) or focus forming units/ml (ffu/ml). In some aspects, the powder pharmaceutical composition has virus or bacteriophage particles that have lost less than 3.5 log titer (in plaque forming units/ml (PFU/mL) or focus forming units/ml (ffu/ml)) as compared to the titer in the precursor solution. In some aspects, the powder pharmaceutical composition has virus or bacteriophage particles that have lost less than 3.0, 2.5, 2.0, 1.5, 1.0 or 0.5 log titer (in PFU/mLor FFU/ml) as compared to the titer in the precursor solution. In some aspects, the temperature in step (b) is about −40° C. to −150° C., −50° C. to −125° C., −55° C. to −100° C. or −65° C. to −75° C. In some aspects, the temperature in step (b) is about −40° C. to −100° C., −40° C. to −90° C., −40° C. to −80° C. or −50° C. to −75° C. In some aspects, the precursor solution comprises leucine. In some aspects, the precursor solution comprises leucine and sucrose. In some aspects, the precursor solution comprises sucrose and leucine in a ratio of about 50:50 to 95:5; 60:40; 70:30 to 90:10; or 75:25 to 80:20 (sucrose:leucine). In some aspects, the powder pharmaceutical composition has a geometric particle size distribution Dv50, measured by dry Rodos method, of less than 15 μm. In some aspects, the powder pharmaceutical composition has a geometric particle size distribution Dv50, measured by dry Rodos method, of less than about 20 μm, 15 μm or 12 μm. In some aspects, at least 20% of the particles have a size of 1-5 μm. In some aspects, at least 25%, 30%, 35%, 40%, 45% or 50% of the particles have a size of 1-5 μm. In some aspects, the precursor solution comprises a pH buffering agent. In some aspects, the pH buffering agent is a PBS or SM buffer. In some aspects, the pH buffering agent is SM buffer and the precursor solution comprises trehalose and leucine.

In some aspects, the biologically active polynucleotide molecules comprise polynucleotide molecules encapsulated in a lipid nanoparticles (LNPs). In some aspects, the biologically active polynucleotide molecule comprises a mRNA. In some aspects, the LNPs comprise ionizable lipids, phospholipids, cholesterol, lecithin and/or poly-(ethylene) glycol (PEG)-lipid. In some aspects, the LNPs have an average particle size of between about 25 nm and 1000 nm, 50 nm and 1000 nm; 50 nm and 600 nm, or 80 nm and 200 nm. In some aspects, the precursor solution comprises about 10% to 30% or 15% to 25% lactose, trehalose, sucrose, mannitol or sorbitol. In some aspects, the biologically active polynucleotide molecule comprises siRNA. In some aspects, the siRNA is less than 30 nucleotides in length. In some aspects, the biologically active polynucleotide molecules comprise polynucleotide molecules complexed with chitosan. In some aspects, the chitosan is PEGylated. In some aspects, LNP comprises DNA molecules complexed with chitosan.

In some aspects, the biologically active polynucleotide molecules comprise genomic material. In some aspects, the biologically active polynucleotide molecules are comprised in intact cells. In some aspects, the intact cells comprise living cells. In some aspects, the intact cells comprise intact bacterial, eukaryotic or archaeal cells. In some aspects, the intact cells comprise intact bacterial cells. In some aspects, the intact cells comprise living bacterial cells. In some aspects, the first excipient comprises a sugar or sugar alcohol. In some aspects, the first excipient comprises lactose, trehalose, sucrose, mannitol or sorbitol. In some aspects, the first excipient comprises sucrose. In some aspects, the surface, onto which materials are deposited, is rotating. In some aspects, the solvent is removed at reduced pressure. In some aspects, the solvent is removed via lyophilization. In some aspects, the lyophilization is carried out at a lyophilization temperature from about −20° C. to about −100° C. In some aspects, the lyophilization temperature is about −40° C. In some aspects, the reduced pressure is less than 400 mTor; 350 mTorr; 300 mTorr or 250 mTorr. In some aspects, the reduced pressure is about 100 mTorr. In some aspects, the method is a GMP method.

In other embodiments, the present disclosure provides pharmaceutical compositions prepared according to the methods of the present disclosure.

In still other embodiments, the present disclosure provides methods of treating a lung disease, lung injury, or lung infection comprising administering an effective amount of a composition of the present disclosure or a composition produced by the methods of the present disclosure to a subject. In some aspects, the lung disease is interstitial lung diseases, chronic obstructive pulmonary disease (COPD), asthma, cystic fibrosis (CF), pulmonary fibrosis or primary ciliary dyskinesia (PCD). In some aspects, the lung infection is a bacterial lung infection. In some aspects, the comprises bacteriophage. In some aspects, the composition comprises LNPs. In some aspects, the composition comprises siRNA.

In yet other embodiments, the present disclosure provides methods of stimulating an immune response in a subject comprising administering an effective amount of a composition of the present disclosure or a composition produced by the methods of the present disclosure to a subject, wherein the biologically active polynucleotide molecules encode an antigen. In some aspects, the composition comprises LNPs and mRNA.

In other embodiments, the present disclosure provides methods of treating a disease in a subject comprising administering an effective amount of a composition of the present disclosure or a composition produced by the methods of the present disclosure to the subject. In some aspects, the disease is a genetic disease. In some aspects, the disease is a lung disease. In some aspects, the disease is an infection.

In still other embodiments, the present disclosure provides methods of treating a disease in a subject comprising: (i) reconstituting a composition of the present disclosure or a composition produced by the methods of the present disclosure, in a pharmaceutically acceptable vehicle; and (ii) administering an effective amount of the reconstituted composition to the subject.

Other objects, features and advantages of the present invention will become apparent from the following detailed description. It should be understood, however, that the detailed description and the specific examples, while indicating certain embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific embodiments presented herein.

FIG. 1 shows titer loss of T7 after thin film freeze-dried with different excipient matrices. Note: the two segments of Y-axis were not in the same scale.

FIG. 2 shows geometric particle size distribution of different TFFD phage formulations.

FIG. 3 shows titer loss of T7 after thin film freeze-dried with various excipient matrices in different solid contents. Note: the two segments of Y-axis were not in the same scale.

FIG. 4 shows geometric particle size distribution of TFFD processed phage formulations with different solid contents.

FIG. 5 shows titer loss of T7 after thin film freeze-dried at different temperatures.

FIG. 6 shows geometric particle size distribution of TFFD phage formulations processed at different temperatures.

FIG. 7 shows titer loss of T7 after thin film freeze-dried in formulations with different initial phage concentration. Note: 5E10, 5E09, 5E08, 5E07, and 5E06 are alternative expressions of 5×10¹⁰ PFU/mL, 5×10⁹ PFU/mL, 5×10⁸ PFU/mL, 5×10⁷ PFU/mL, and 5×10⁶ PFU/mL, respectively.

FIG. 8 shows geometric particle size distribution of TFFD phage formulations processed with different phage concentration. Note: 5E10, 5E09, 5E08, 5E07, and 5E06 are alternative expressions of 5×10¹⁰ PFU/mL, 5×10⁹ PFU/mL, 5×10⁸ PFU/mL, 5×10⁷ PFU/mL, and 5×10⁶ PFU/mL, respectively

FIG. 9 shows titer loss of T7 after thin film freeze-dried in different buffer systems.

FIG. 10 shows geometric particle size distribution of TFFD phage formulations processed with no buffer, PBS buffer, or SM buffer.

FIG. 11 shows titer loss of T7 phage in each step of thin film freeze-drying.

FIG. 12 shows X-ray diffraction patterns of TFFD phage powders.

FIG. 13 shows powder morphology images by scanning electron microscopy.

FIG. 14 shows phage morphology images by transmission election microscopy.

FIG. 15 shows thermogravimetric analysis curves of TFFD phage powders.

FIG. 16 shows water content in TFFD phage powder determined by TGA.

FIG. 17 shows intracellular uptake of LNP formulations at different N/P ratios in HEK-293 cells measured by percent GFP expression (left axis) and fluorescence intensity (right axis).

FIGS. 18A-18D shows characterization of LNP formulations. (FIG. 18A) size, (FIG. 18B) zeta-potential, (FIG. 18C) encapsulation efficiency, and (FIG. 18D) pKa. Stability of the lipid nanoparticles was evaluated by measuring size and zeta-potential at day 1 and after 14 days from preparation and storage at 4° C. (mean±SD, n=3).

FIGS. 19A-19C show stability of LNP formulations before and after nebulization in terms of (a) size, (b) zeta-potential, and (c) encapsulation efficiency. The size (***p=0.0004) and encapsulation efficiency (****p<0.0001) of nebulized formulation were significantly different from pre-nebulized formulations.

FIGS. 20A & 20B show efficiency of intracellular uptake in HEK-293 cells over 16 days after LNPs preparation. (FIG. 20A) percent GFP expression, and (FIG. 20B) fluorescence intensity.

FIGS. 21A-21D show in vitro intracellular uptake in terms of percent GFP expression (FIGS. 21A & 21C) and fluorescence intensity (FIGS. 21B & 21D) of LNP formulations before and after nebulization in HEK-293 and NuLi-1 cells.

FIGS. 22A & 22B show efficacy and biodistribution of F2, F8, F11, F17 formulations with luciferase mRNA. (FIG. 22A) Efficacy of the four lead formulations before and after nebulization in lung as measured in total flux of luminescence 6 h after intratracheal delivery of 15 μg of total mRNA. (FIG. 22B) Representative images of the luciferase expression in lungs, heart, liver, and kidneys measured by IVIS imaging.

FIGS. 23A-23D show correlation between particle size and PEG-lipid. (FIG. 23A) Effect of PEG-lipid molar ratio on particle size before nebulization. (FIG. 23B) Effect of type of PEG-lipid on particle size before nebulization. (FIG. 23C) Effect of PEG-lipid molar ratio on particle size after nebulization. (FIG. 23D) Effect of type of PEG-lipid on particle size after nebulization.

FIGS. 24A-24D show correlation between zeta potential and PEG-lipid. (FIG. 24A) Significant effects of PEG-lipid molar ratio on zeta potential before nebulization. (FIG. 24B) Significant effects of type of PEG-lipid on zeta potential before nebulization. (FIG. 24C) Significant effects of PEG-lipid molar ratio on zeta potential after nebulization. (FIG. 24D) Significant effects of type of PEG-lipid on zeta potential after nebulization.

FIGS. 25A-25D show correlation of encapsulation efficiency and cholesterol molar ratio & type of phospholipid. (FIG. 25A) Significant correlation (p<0.05) between encapsulation efficiency and cholesterol molar ratio before nebulization. (FIG. 25B) No significant effects (p>0.05) of type of phospholipid on encapsulation efficiency before nebulization. (FIG. 25C) No significant correlation between encapsulation efficiency and cholesterol molar ratio after nebulization. (FIG. 25D) Significant effects of type of phospholipid on encapsulation efficiency after nebulization. **p<0.01.

FIGS. 26A-26F show correlation analysis between intracellular uptake (percent GFP expression and fluorescence intensity) and PEG-lipid molar ratio or type of phospholipid. (FIG. 26A) Significant effect of PEG-lipid molar ratio on percent GFP expression before nebulization. (FIG. 26B) Significant effect of type of phospholipid on percent GFP expression before nebulization. (FIG. 26C) Significant effect of PEG-lipid molar ratio on percent GFP expression before nebulization. (FIG. 26D) Significant effect of PEG-lipid molar ratio on percent GFP expression after nebulization. (FIG. 26E) No significant effect of type of phospholipid on percent GFP expression after nebulization. (FIG. 26F) Significant effect of PEG-lipid molar ratio on fluorescence intensity after nebulization.

FIGS. 27A-27H show orthogonal trends of intracellular uptake in terms of percent GFP expression and fluorescence intensity, whereby dotted line represented non-significance and solid line represented significance. (FIGS. 27A-27D): Correlation between intracellular uptake and formulation properties before nebulization. (FIGS. 27E-27H): Correlation between intracellular uptake and formulation properties after nebulization.

FIGS. 28A-28C show characterization of LNP formulations. (FIG. 28A) size, (FIG. 28B) zeta-potential, and (FIG. 28C) encapsulation efficiency.

FIGS. 29A-29D show in vitro intracellular uptake in terms of percent GFP expression (FIGS. 29A & 29B) and fluorescence intensity (FIGS. 29C & 29D) of LNP formulations in HEK-293 and NuLi-1 cells.

FIGS. 30A-30F show macroscopic appearance of 42 dry powder formulations. (FIG. 30A) formulations containing mannitol, (FIG. 30B) formulations containing mannitol and leucine, (FIG. 30C) formulations containing sucrose, (FIG. 30D) formulations containing sucrose and leucine, (FIG. 30E) formulations containing trehalose, (FIG. 30F) formulations containing trehalose and leucine.

FIGS. 31A-31F show size, PDI and zeta potential of reconstituted dry powder formulations. (FIG. 31A) size and PDI of reconstituted TFF formulations containing mannitol with/without leucine, (FIG. 31B) size and PDI of reconstituted formulations containing sucrose with/without leucine, (FIG. 31C) size and PDI of reconstituted TFF formulations containing trehalose with/without leucine, (FIG. 31D) zeta potential of reconstituted TFF formulations containing mannitol with/without leucine, (FIG. 31E) zeta potential of reconstituted TFF formulations containing sucrose with/without leucine, (FIG. 31E) zeta potential of reconstituted TFF formulations containing trehalose with/without leucine.

FIG. 32 shows transfection efficiency of reconstituted formulations.

FIG. 33 shows structure of nanocomplexes.

FIG. 34 shows scanning electron microscopy images of six refined dry powder formulations.

FIGS. 35A-35C shows X-ray diffraction patterns of six refined dry powder formulations and raw mannitol, sucrose, and trehalose.

FIG. 36 shows aerodynamic particle size distribution profile of refined TFF formulations.

FIG. 37 shows Z-average size of LNP.

FIG. 38 shows transfection efficiency of LNP-mRNA dry powder formulations in HEK-293 cells.

FIGS. 39A & 39B show representative SEM micrographs of dry powders of SLNs.

FIG. 39A: spray dried SLNs; FIG. 39B: SLNs prepared by TFFD. Top images were obtained with 3K magnification (scale bar: 10 μm) and bottom images with 10.5K magnification (scale bar: 2 μm).

FIG. 40 shows deposition patterns of spray dried vs. thin-film freeze-dried (TFFD) SLNs with mannitol as the excipient. Data are mean±SD (n=3).

FIGS. 41A & 41B show a representative SEM image of thin-film freeze-dried siRNA-SLNs (FIG. 41A). (FIG. 41B) Deposition pattern of siRNA-SLNs in different stages of the Next Generation Impactor. Data are mean±SD (n=3).

FIG. 42 shows down-regulation of TNF-α release from J774A.1 cells by TNF-α-siRNA-SLNs, before (i.e. suspension) and after they were subjected to TFFD (i.e. Powder). TNF-α-siRNA complexed with Lipofectamine was used a control. Data rare mean±SD (n=4). Groups labeled with a, b, and d are different from groups labeled in c (p<0.05).

FIG. 43 shows penetration of the siRNA-SLNs through simulated mucus. Data are mean±SD (n=3).

FIG. 44 shows evaluation of the function of the TFN-α siRNA in down-regulating TNF-α release.

FIG. 45 shows Next Gen impaction data for TopFluor-cholesterol labeled solid lipid nanoparticles dry powder. The fraction of nanoparticles recovered from each stage in the NGI is plotted. MOC is the micro-orifice collector and IP is the induction port. Error bars are the standard deviation for two trials.

FIGS. 46A & 46B show physical characterization of the acid-sensitive-TNF-α siRNA-SLNs. (FIG. 46A) TEM image of the SLN. (FIG. 46B) in vitro release of the fluorescently labeled siRNA from acid-sensitive-TNF-α siRNA-SLNs.

FIG. 47 shows physical appearance of the SLN dry powder.

FIG. 48 shows SEM images of spray dried (left) and freeze dried (right) SLN powder.

FIG. 49 show NGI deposition profile for spray-dried SLNs and freeze-dried SLNs. NGI data was collected over three independent trials and had recovery over 90%.

FIG. 50 shows comparison of SLNs size distribution before and after drying using freeze drying (left) and spray drying (right).

FIG. 51 shows a comparison of the morphology of shelf freeze-dried bacterial powder and thin-film freeze-dried bacterial powders. Left: shelf freeze-dried bacteria powder, with sucrose (10% w/v) as cryoprotectant; Right: TFFD bacteria powder with mannitol (250 μL of 5% w/w) as cryoprotectant.

FIG. 52 shows deposition profiles of thin-film freeze-dried plasmid powders in various stages after applied to NGI using Plastiape® RS00 high-resistance DPI at a flow rate of 60 L/min. Data are mean±S.D. (n=3)

FIGS. 53A-53C show representative SEM images of thin-film freeze-dried pCMV-β powder (formulation P3).

FIG. 54 shows the gel electrophoresis analysis of the plasmid before and after TFF formulation. Lane 1: pCMV-beta, Formulation 7; Lane 2: pCMV-beta, Formulation 7, Hind III & EcoR1; Lane 3: pCMV-beta, Formulation 7, EcoR I; Lane 4: GeneRuler 1 kb Plus DNA Ladder (ThermoFisher); Lane 5: pCMV-beta, Formulation 7 after TFFD; Lane 6: pCMV-beta, Formulation 7 after TFFD, Hind III & EcoR I; Lane 7: pCMV-beta, Formulation 7 after TFFD, EcoR I; Lane 8: pCMV-beta, Hind III & EcoR I; and Lane 9: pCMV-beta, EcoR I. pCMV-beta, lanes 1 and 5, loaded 500 ng of plasmid, Others, −420 ng. Digested time: 2 h, EcoR I: 7.3 kbp and Hind III and EcoR I: 4.6 plus 2.7.

FIG. 55 shows a representative TEM image of mRNA-LNPs after they were subjected to thin-film freeze-drying (formulation 5) and reconstitution.

### DESCRIPTION OF ILLUSTRATIVE EMBODIMENTS

### I. The Present Embodiments

Provided herein are dry powder formulations of biologically active polynucleotides that can be made by a URF process. It was shown that, by the use of URF, the compositions can be stabilized such that the polynucleotides are protected from excessive degradation and components retain substantial biological activity after formulation. In some cases, formulations include at least first excipient, such as sugar, to provide yet further stabilization. Thus, dry powders of the embodiments can comprise a wide variety of polynucleotide-containing compositions. Moreover, it has been demonstrated that the powders of the embodiments can be used to directly administer therapeutic agents, e.g., to the lungs. Thus, the aspects of the present invention provide new pharmaceutical formulations, formulation methods and administration modalities that demonstrate significant advantages over previously compositions and methods that have been used.

In some cases, a powder of the embodiment comprises viruses, such a bacteriophage. It has been shown that viruses processed into powders as detailed herein are able to retain substantial virus titer. Thus, methods and compositions provided herein can be used to stabilize virus, such as for storage and/or transportation. Likewise, virus-containing powders can be directly administered to patients in need thereof (or reconstituted prior to administration). For example, the virus may be an attenuated virus or virus like particles and the composition used as a vaccine to stimulate and immune response. In further aspects, the virus can be a bacteriophage and be used to treat a bacterial infection, such a lung infection. In still further aspects a virus can be gene therapy vector, for use in disease treatment.

In some cases, powders of the embodiments can comprise single stranded or double stranded RNA or DNA. Such polynucleotides can be encapsulated in or in complex with nanoparticles, such a lipid nanoparticle. For example, in some cases, polynucleotides, such as mRNAs or siRNAs are provided in complex with LNPs. For example, a mRNA-LNP complex can encode a therapeutically active protein (e.g., for gene replacement therapy) or an antigen (e.g., for vaccination). In preferred aspects, the LNP provided in dry powders of the embodiments are formed from multiple lipid types, such as cationic lipids, phospholipids and/or PEGylated lipids. In further aspects, a RNA-LNP powder further comprises at least a first excipient, such as sugar or amino acid. In some aspects, dry powders can be directly administered (e.g., by dispersion in the lungs) to subjects to treat a disease or stimulate an immune response.

In still further aspects, powders are provided with LNPs comprising siRNA. It has been demonstrated that such compositions provide a stabilized formulation that is also ideal for delivery, e.g., such as by dispersion of the powder to the lungs. Thus, siRNAs could be employed to treat a wide range of disease. For example, in the case of an over-active or aberrant immune response, siRNA could target a gene that stimulates inflammatory immune response, such a TNF-alpha. In further aspects, siRNA could be targeted to oncogenes or genes of pathogens for disease treatment.

In further aspects, polynucleotides, such as DNA, as provided in powders in complex with chitosan nanoparticles. In some aspects, the chitosan nanoparticles are further modified by PEGylation. Such DNA molecules, can be, e.g., plasmids or DNA expression vectors. In some cases, DNA can encode a CRISPR system, to provide targeted gene replacement ins a subject. Thus system, for example, is ideal for the treatment of genetic diseases, such as cystic fibrosis. In some aspects, DNA-complex containing powders can be directly administered (e.g., by dispersion in the lungs) to subjects to treat a disease.

In still further aspects, dry powder compositions of the embodiments comprise intact cells. For example, the powders can comprise eukaryotic or bacterial cells. In particular, it has been demonstrated herein that living cells can be formulated into URF powders and that such powders retain a high level of cell viability. Thus, dry powders can be used to stabilize, store and/or transport intact or living cells, such as bacterial cells. Such compositions have a wide range of potential uses. For example, attenuated or inactivated bacteria could be formulated and used to stimulate immune responses. Alternatively, beneficial bacterial could be formulated to provide probiotic compositions. Moreover cell-containing dry powders can serve as means for directly delivering cells to patients as oral and/or aerosol formulations. In some aspects, bacteria-containing dry powder may have applications in agriculture, such as a stabilized biocontrol agent. Thus, in some case, bacteria-containing powders can be aerosolized and applied to a field, e.g., of crops.

### II. Ultra-Rapid Freezing (URF) Formulation

In certain aspects, the present disclosure provides pharmaceutical compositions which may be prepared using a URF process, such as thin-film freezing process. Such methods are described in U.S. Patent Application No. 2010/0221343 and Watts, et al., 2013, both of which are incorporated herein by reference. In some cases, the methods employ an ultra-rapid freezing rate of up to 10,000 K/sec, e.g., at least 1,000, 2,000, 5,000 or 8,000 K/sec. In some embodiments, these methods involve dissolving the components of the pharmaceutical composition into a solvent to form a precursor solution. The solvents may be either water or an organic solvent. However, the in preferred aspects, the precursor solution is an aqueous solution that includes at least a first excipient and biologically active polynucleotide molecules. In some embodiments, the precursor solution may contain less than 10% w/v of the therapeutic agent and excipient. The precursor solution may contain less than 0.5%, 1%, 2%, 3%, 4%, 5%, 6%, 7%, 8%, 9%, or 10% w/v, or any range derivable therein.

This precursor solution may be deposited on a surface which is at a temperature that causes the precursor solution to freeze. In some embodiments, this temperature may be below the freezing point of the solution at ambient pressure. In other embodiments, a reduced pressure may be applied to the surface causing the solution to freeze at a temperature below the ambient pressure's freezing point. The surface may also be rotating or moving on a moving conveyer-type system thus allowing the precursor solution to distribute evenly on the surface. Alternatively, the precursor solution may be applied to surface in such a manner to generate an even surface.

After the precursor solution has been applied to the surface, the solvent may be removed to obtain a pharmaceutical composition. Any appropriate method of removing the solvent may be applied including evaporation under reduced pressure or elevated temperature or lyophilization. In some embodiments, the lyophilization may comprise a reduced pressure and/or a reduced temperature. Such a reduced temperature may be from 25° C. to about −200° C., from 20° C. to about −175° C., from about 20° C. to about −150° C., from 0° C. to about −125° C., from 20° C. to about −100° C., from −75° C. to about −175° C., or from ˜100° C. to about −160° C. The temperature is from about −20° C., −30° C., −35° C., −40° C., −45° C., −50° C., −55° C., −60° C., −70° C., −80° C., −90° C., −100° C., −110° C., −120° C., −130° C., −140° C., −150° C., −160° C., −170° C., 180° C., −190° C., to about −200° C., or any range derivable therein. Additionally, the solvent may be removed at a reduced pressure of less than 500 mTorr, 450 mTorr, 400 mTorr, 375 mTorr, 350 mTorr, 325 mTorr, 300 mTorr, 275 mTorr, 250 mTorr, 225 mTorr, 200 mTorr, 175 mTorr, 150 mTorr, 125 mTorr, 100 mTorr, 75 mTorr, 50 mTorr, or 25 mTorr.

Such as composition prepared using these methods may exhibit a brittle nature such that the composition is easily sheared into smaller particles when processed through a device. These compositions have high surface areas as well as exhibit improved flowability of the composition. Such flowability may be measured, for example, by the Carr index or other similar measurements. In particular, the Carr's index may be measured by comparing the bulk density of the powder with the tapped density of the powder. Such compounds may exhibit a favorable Carr index and may result in the particles being better sheared to give smaller particles when the composition is processed through a secondary device to further process a powder composition.

### III. Components of Compositions of the Embodiments

A. Composition Including Biologically Active Polynucleotides

Methods and composition of the embodiments concern biologically active polynucleotides. In some cases these can comprise single stranded or double stranded RNA or DNA. Such polynucleotides can be encapsulated in or in complex with nanoparticles. For example, in some cases polynucleotides, such as mRNAs or siRNAs are provided in complex with LNPs. In further aspects, polynucleotides, such as DNA, as provided in complex with chitosan nanoparticles. In still further aspects, biologically active polynucleotides are provided in viruses, such as bacteriophage, or virus like particles. In yet further aspects, biologically active polynucleotides are provided in intact cells, such as living bacterial cells.

In some aspects, a nucleic acid molecule of the embodiments encodes a therapeutic polypeptide. For example, the therapeutic protein may be a protein, such as an enzyme that is non-functional or disrupted in a particular disease state (e.g., CFTR in cystic fibrosis).

In further aspects, a polynucleotide of the embodiments encodes an antigen, such as an antigen from a pathogen or a cancer cell-associated antigen. For example, the cancer associated antigen can be CD19, CD20, ROR1, CD22, carcinoembryonic antigen, alphafetoprotein, CA-125, 5T4, MUC-1, epithelial tumor antigen, prostate-specific antigen, melanoma-associated antigen, mutated p53, mutated ras, HER2/Neu, folate binding protein, GD2, CD123, CD33, CD138, CD23, CD30, CD56, c-Met, mesothelin, GD3, HERV-K, IL-11Ralpha, kappa chain, lambda chain, CSPG4, ERBB2, EGFRvIII or VEGFR2. In some specific aspects the antigen is GP240, 5T4, HER1, CD-33, CD-38, VEGFR-1, VEGFR-2, CEA, FGFR3, IGFBP2, IGF-1R, BAFF-R, TACI, APRIL, Fn14, ERBB2 or ERBB3

Antigens useful in the present disclosure may include those derived from viruses including, but not limited to, those from the family Arenaviridae (e.g., Lymphocytic choriomeningitis virus), Arterivirus (e.g., Equine arteritis virus), Astroviridae (Human astrovirus 1), Birnaviridae (e.g., Infectious pancreatic necrosis virus, Infectious bursal disease virus), Bunyaviridae (e.g., California encephalitis virus Group), Caliciviridae (e.g., Caliciviruses), Coronaviridae (e.g., Human coronaviruses 299E and OC43), Deltavirus (e.g., Hepatitis delta virus), Filoviridae (e.g., Marburg virus, Ebola virus), Flaviviridae (e.g., Yellow fever virus group, Hepatitis C virus), Hepadnaviridae (e.g., Hepatitis B virus), Herpesviridae (e.g., Epstein-Bar virus, Simplexvirus, Varicellovirus, Cytomegalovirus, Roseolovirus, Lymphocryptovirus, Rhadinovirus), Orthomyxoviridae (e.g., Influenzavirus A, B, and C), Papovaviridae (e.g., Papillomavirus), Paramyxoviridae (e.g., Paramyxovirus such as human parainfluenza virus 1, Morbillivirus such as Measles virus, Rubulavirus such as Mumps virus, Pneumovirus such as Human respiratory syncytial virus), Picornaviridae (e.g., Rhinovirus such as Human rhinovirus 1A, Hepatovirus such Human hepatitis A virus, Human poliovirus, Cardiovirus such as Encephalomyocarditis virus, Aphthovirus such as Foot-and-mouth disease virus O, Coxsackie virus), Poxyiridae (e.g., Orthopoxvirus such as Variola virus or monkey poxvirus), Reoviridae (e.g., Rotavirus such as Groups A-F rotaviruses), Retroviridae (Primate lentivirus group such as human immunodeficiency virus 1 and 2), Rhabdoviridae (e.g., rabies virus), Togaviridae (e.g., Rubivirus such as Rubella virus), Human T-cell leukemia virus, Murine leukemia virus, Vesicular stomatitis virus, Wart virus, Blue tongue virus, Sendai virus, Feline leukemia virus, Simian virus 40, Mouse mammary tumor virus, Dengue virus, HIV-1 and HIV-2, West Nile, H1N1, SARS, 1918 Influenza, Tick-borne encephalitis virus complex (Absettarov, Hanzalova, Hypr), Russian Spring-Summer encephalitis virus, Congo-Crimean Hemorrhagic Fever virus, Junin Virus, Kumlinge Virus, Marburg Virus, Machupo Virus, Kyasanur Forest Disease Virus, Lassa Virus, Omsk Hemorrhagic Fever Virus, FIV, SIV, Herpes simplex 1 and 2, Herpes Zoster, Human parvovirus (B19), Respiratory syncytial virus, Pox viruses (all types and serotypes), Coltivirus, Reoviruses—all types, and/or Rubivirus (rubella).

Antigens useful in the present disclosure may include those derived from bacteria including, but not limited to, Streptococcus agalactiae, Legionella pneumophilia, Streptococcus pyogenes, Escherichia coli, Neisseria gonorrhosae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Candida albicans, Cryptococcus neoformans, Histoplasma capsulatum, Coccidioides immitis, Blastomyces dermatitidis, Aspergillus fumigatus, Penicillium marneffei, Bacillus anthracis, Bartonella, Bordetella pertussis, Brucella—all serotypes, Chlamydia trachomatis, Chlamydia pneumoniae, Clostridium botulinum—anything from clostridium serotypes, Haemophilus influenzae, Helicobacter pylori, Klebsiella—all serotypes, Legionella—all serotypes, Listeria, Mycobacterium—all serotypes, Mycoplasma—human and animal serotypes, Rickettsia—all serotypes, Shigella—all serotypes, Staphylococcus aureus, Streptococcus—S. pneumoniae, S. pyogenes, Vibrio cholera, Yersinia enterocolitica, and/or Yersinia pestis.

Antigens useful in the present disclosure may include those derived from parasites including, but not limited to, Ancylostomahuman hookworms, Leishmania—all strains, Microsporidium, Necator human hookworms, Onchocerca filarial worms, Plasmodium—all human strains and simian species, Toxoplasma—all strains, Trypanosoma—all serotypes, and/or Wuchereria bancrofti filarial worms.

(1) DNA Molecules

In certain aspects, a nucleic acid for delivery in accordance with the embodiments is a DNA molecule. For example, the DNA molecule may be an expression vector. The term “expression vector” refers to any type of genetic construct comprising a nucleic acid coding for a RNA capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host cell. In addition to control sequences that govern transcription and translation, vectors and expression vectors may contain nucleic acid sequences that serve other functions. In some aspects, a DNA expression vector may encode a therapeutic polypeptide or an antigen polypeptide. In further aspects, a DNA expression vector an encode the elements of CRISPR system.

CRISPR Systems

Clustered regularly interspaced short palindromic repeats (CRISPR) and CRISPR-associated (Cas) proteins can be used in accordance with the embodiments for targeted gene disruption and/or replacement. In general, “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), and/or other sequences and transcripts from a CRISPR locus.

The CRISPR/Cas nuclease or CRISPR/Cas nuclease system can include a non-coding RNA molecule (guide) RNA, which sequence-specifically binds to DNA, and a Cas protein (e.g., Cas9), with nuclease functionality (e.g., two nuclease domains). One or more elements of a CRISPR system can derive from a type I, type II, or type III CRISPR system, e.g., derived from a particular organism comprising an endogenous CRISPR system, such as Streptococcus pyogenes.

In some aspects, a Cas nuclease and gRNA (including a fusion of crRNA specific for the target sequence and fixed tracrRNA) are introduced into the cell. In general, target sites at the 5′ end of the gRNA target the Cas nuclease to the target site, e.g., the gene, using complementary base pairing. The target site may be selected based on its location immediately 5′ of a protospacer adjacent motif (PAM) sequence, such as typically NGG, or NAG. In this respect, the gRNA is targeted to the desired sequence by modifying the first 20, 19, 18, 17, 16, 15, 14, 14, 12, 11, or 10 nucleotides of the guide RNA to correspond to the target DNA sequence. In general, a CRISPR system is characterized by elements that promote the formation of a CRISPR complex at the site of a target sequence. Typically, “target sequence” generally refers to a sequence to which a guide sequence is designed to have complementarity, where hybridization between the target sequence and a guide sequence promotes the formation of a CRISPR complex. Full complementarity is not necessarily required, provided there is sufficient complementarity to cause hybridization and promote formation of a CRISPR complex.

The CRISPR system can induce double stranded breaks (DSBs) at the target site, followed by disruptions as discussed herein. In other embodiments, Cas9 variants, deemed “nickases,” are used to nick a single strand at the target site. Paired nickases can be used, e.g., to improve specificity, each directed by a pair of different gRNAs targeting sequences such that upon introduction of the nicks simultaneously, a 5′ overhang is introduced. In other embodiments, catalytically inactive Cas9 is fused to a heterologous effector domain such as a transcriptional repressor or activator, to affect gene expression.

The target sequence may comprise any polynucleotide, such as DNA or RNA polynucleotides. The target sequence may be located in the nucleus or cytoplasm of the cell, such as within an organelle of the cell. Generally, a sequence or template that may be used for recombination into the targeted locus comprising the target sequences is referred to as an “editing template” or “editing polynucleotide” or “editing sequence”. In some aspects, an exogenous template polynucleotide may be referred to as an editing template. In some aspects, the recombination is homologous recombination.

Typically, in the context of an endogenous CRISPR system, formation of the CRISPR complex (comprising the guide sequence hybridized to the target sequence and complexed with one or more Cas proteins) results in cleavage of one or both strands in or near (e.g. within 1, 2, 3, 4, 5, 6, 7, 8, 9, 10, 20, 50, or more base pairs from) the target sequence. The tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of the CRISPR complex, such as by hybridization along at least a portion of the tracr sequence to all or a portion of a tracr mate sequence that is operably linked to the guide sequence. The tracr sequence has sufficient complementarity to a tracr mate sequence to hybridize and participate in formation of the CRISPR complex, such as at least 50%, 60%, 70%, 80%, 90%, 95% or 99% of sequence complementarity along the length of the tracr mate sequence when optimally aligned.

One or more vectors driving expression of one or more elements of the CRISPR system can be introduced into the cell such that expression of the elements of the CRISPR system direct formation of the CRISPR complex at one or more target sites. Components can also be delivered to cells as proteins and/or RNA. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. The vector may comprise one or more insertion sites, such as a restriction endonuclease recognition sequence (also referred to as a “cloning site”). In some embodiments, one or more insertion sites are located upstream and/or downstream of one or more sequence elements of one or more vectors. When multiple different guide sequences are used, a single expression construct may be used to target CRISPR activity to multiple different, corresponding target sequences within a cell.

A vector may comprise a regulatory element operably linked to an enzyme-coding sequence encoding the CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

The CRISPR enzyme can be Cas9 (e.g., from S. pyogenes or S. pneumonia). The CRISPR enzyme can direct cleavage of one or both strands at the location of a target sequence, such as within the target sequence and/or within the complement of the target sequence. The vector can encode a CRISPR enzyme that is mutated with respect to a corresponding wild-type enzyme such that the mutated CRISPR enzyme lacks the ability to cleave one or both strands of a target polynucleotide containing a target sequence. For example, an aspartate-to-alanine substitution (D10A) in the RuvC I catalytic domain of Cas9 from S. pyogenes converts Cas9 from a nuclease that cleaves both strands to a nickase (cleaves a single strand). In some embodiments, a Cas9 nickase may be used in combination with guide sequence(s), e.g., two guide sequences, which target respectively sense and antisense strands of the DNA target. This combination allows both strands to be nicked and used to induce NHEJ or HDR.

In some embodiments, an enzyme coding sequence encoding the CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human primate. In general, codon optimization refers to a process of modifying a nucleic acid sequence for enhanced expression in the host cells of interest by replacing at least one codon of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization.

In general, a guide sequence is any polynucleotide sequence having sufficient complementarity with a target polynucleotide sequence to hybridize with the target sequence and direct sequence-specific binding of the CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more.

Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), Clustal W, Clustal X, BLAT, Novoalign (Novocraft Technologies, ELAND (Illumina, San Diego, Calif.), SOAP (available at soap.genomics.org.cn), and Maq (available at maq.sourceforge.net).

The CRISPR enzyme may be part of a fusion protein comprising one or more heterologous protein domains. A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-5-transferase (GST), horseradish peroxidase (HRP), chloramphenicol acetyltransferase (CAT) beta galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4A DNA binding domain fusions, and herpes simplex virus (HSV) BP16 protein fusions. Additional domains that may form part of a fusion protein comprising a CRISPR enzyme are described in US 20110059502, incorporated herein by reference.

(2) Inhibitory Nucleic Acid Molecules

Small inhibitory nucleic acid (siNA e.g., siRNA) are well known in the art. For example, siRNA and double-stranded RNA have been described in U.S. Pat. Nos. 6,506,559 and 6,573,099, as well as in U.S. Patent Applications 2003/0051263, 2003/0055020, 2004/0265839, 2002/0168707, 2003/0159161, and 2004/0064842, all of which are herein incorporated by reference in their entirety.

Within a siNA, the components of a nucleic acid need not be of the same type or homogenous throughout (e.g., a siNA may comprise a nucleotide and a nucleic acid or nucleotide analog). Typically, siNA form a double-stranded structure; the double-stranded structure may result from two separate nucleic acids that are partially or completely complementary. In certain embodiments of the present invention, the siNA may comprise only a single nucleic acid (polynucleotide) or nucleic acid analog and form a double-stranded structure by complementing with itself (e.g., forming a hairpin loop). The double-stranded structure of the siNA may comprise 16, 20, 25, 30, 35, 40, 45, 50, 60, 65, 70, 75, 80, 85, 90, 100, 150, 200, 250, 300, 350, 400, 450, 500 or more contiguous nucleobases, including all ranges therein. The siNA may comprise 17 to 35 contiguous nucleobases, more preferably 18 to 30 contiguous nucleobases, more preferably 19 to 25 nucleobases, more preferably 20 to 23 contiguous nucleobases, or 20 to 22 contiguous nucleobases, or 21 contiguous nucleobases that hybridize with a complementary nucleic acid (which may be another part of the same nucleic acid or a separate complementary nucleic acid) to form a double-stranded structure.

Agents of the present embodiments useful for practicing the methods of the present invention include, but are not limited to siRNAs. Typically, introduction of double-stranded RNA (dsRNA), which may alternatively be referred to herein as small interfering RNA (siRNA), induces potent and specific gene silencing, a phenomena called RNA interference or RNAi. This phenomenon has been extensively documented in the nematode C. elegans (Fire et al., 1998), but is widespread in other organisms, ranging from trypanosomes to humans. Depending on the organism being discussed, RNA interference has been referred to as “cosuppression,” “post-transcriptional gene silencing,” “sense suppression,” and “quelling.” RNAi is an attractive biotechnological tool because it provides a means for knocking out the activity of specific genes.

In designing RNAi there are several factors that need to be considered, such as the nature of the siRNA, the durability of the silencing effect, and the choice of delivery system. To produce an RNAi effect, the siRNA that is introduced into the organism will typically contain exonic sequences. Furthermore, the RNAi process is homology dependent, so the sequences must be carefully selected so as to maximize gene specificity, while minimizing the possibility of cross-interference between homologous, but not gene-specific sequences. Preferably the siRNA exhibits greater than 80%, 85%, 90%, 95%, 98%, or even 100% identity between the sequence of the siRNA and the gene to be inhibited. Sequences less than about 80% identical to the target gene are substantially less effective. Thus, the greater homology between the siRNA and the gene to be inhibited, the less likely expression of unrelated genes will be affected.

In addition, the size of the siRNA is an important consideration. In some embodiments, the present invention relates to siRNA molecules that include at least about 19-25 nucleotides and are able to modulate gene expression. In the context of the present invention, the siRNA is preferably less than 500, 200, 100, 50, or 25 nucleotides in length. More preferably, the siRNA is from about 19 nucleotides to about 25 nucleotides in length.

A target gene generally means a polynucleotide comprising a region that encodes a polypeptide, or a polynucleotide region that regulates replication, transcription, or translation or other processes important to expression of the polypeptide, or a polynucleotide comprising both a region that encodes a polypeptide and a region operably linked thereto that regulates expression. The targeted gene can be chromosomal (genomic) or extrachromosomal. It may be endogenous to the cell, or it may be a foreign gene (a transgene). The foreign gene can be integrated into the host genome or it may be present on an extrachromosomal genetic construct such as a plasmid or a cosmid. The targeted gene can also be derived from a pathogen, such as a virus, bacterium, fungus, or protozoan, which is capable of infecting an organism or cell. Target genes may be viral and pro-viral genes that do not elicit the interferon response, such as retroviral genes. The target gene may be a protein-coding gene or a non-protein coding gene, such as a gene that codes for ribosomal RNAs, spliceosomal RNA, tRNAs, etc.

Any gene being expressed in a cell can be targeted. Preferably, a target gene is one involved in or associated with the progression of cellular activities important to disease or of particular interest as a research object. Thus, by way of example, the following are classes of possible target genes that may be used in the methods of the present invention to modulate or attenuate target gene expression: developmental genes (e.g., adhesion molecules, cyclin kinase inhibitors, Wnt family members, Pax family members, Winged helix family members, Hox family members, cytokines/lymphokines and their receptors, growth or differentiation factors and their receptors, neurotransmitters and their receptors), tumor suppressor genes (e.g., APC, CYLD, HIN-1, KRAS2b, p16, p19, p21, p2′7, p27mt, p53, p57, p′73, PTEN, Rb, Uteroglobin, Skp2, BRCA-1, BRCA-2, CHK2, CDKN2A, DCC, DPC4, MADR2/JV18, MEN1, MEN2, MTS1, NF1, NF2, VHL, WRN, WT1, CFTR, C-CAM, CTS-1, zac1, ras, MMAC1, FCC, MCC, FUS1, Gene 26 (CACNA2D2), PL6, Beta* (BLU), Luca-1 (HYAL1), Luca-2 (HYAL2), 123F2 (RASSF1), 101F6, Gene 21 (NPRL2), or a gene encoding a SEM A3 polypeptide), pro-apoptotic genes (e.g., CD95, caspase-3, Bax, Bag-1, CRADD, TSSC3, bax, hid, Bak, MKP-7, PARP, bad, bcl-2, MST1, bbc3, Sax, BIK, and BID), cytokines (e.g., GM-CSF, G-CSF, IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, IL-19, IL-20, IL-21, IL-22, IL-23, IL-24, IL-25, IL-26, IL-27, IL-28, IL-29, IL-30, IL-31, IL-32 IFN-α, IFN-β, IFN-γ, MIP-1α, MIP-1β, TGF-β, TNF-α, TNF-β, PDGF, and mda7), oncogenes (e.g., ABLI, BLC1, BCL6, CBFA1, CBL, CSFIR, ERBA, ERBB, EBRB2, ETS1, ETS1, ETV6, FGR, FOX, FYN, HCR, HRAS, JUN, KRAS, LCK, LYN, MDM2, MLL, MYB, MYC, MYCL1, MYCN, NRAS, PIM1, PML, RET, SRC, TALI, TCL3 and YES), and enzymes (e.g., ACP desaturases and hycroxylases, ADP-glucose pyrophorylases, ATPases, alcohol dehycrogenases, amylases, amyloglucosidases, catalases, cellulases, cyclooxygenases, decarboxylases, dextrinases, esterases, DNA and RNA polymerases, galactosidases, glucanases, glucose oxidases, GTPases, helicases, hemicellulases, integrases, invertases, isomersases, kinases, lactases, lipases, lipoxygenases, lysozymes, nucleases, pectinesterases, peroxidases, phosphatases, phospholipases, phosphorylases, polygalacturonases, proteinases and peptideases, pullanases, recombinases, reverse transcriptases, topoisomerases, xylanases).

siRNA can be obtained from commercial sources, natural sources, or can be synthesized using any of a number of techniques well-known to those of ordinary skill in the art. For example, one commercial source of predesigned siRNA is Ambion®, Austin, Tex. Another is Qiagen® (Valencia, Calif.). An inhibitory nucleic acid that can be applied in the compositions and methods of the present invention may be any nucleic acid sequence that has been found by any source to be a validated downregulator of a protein of interest.

In one aspect, an isolated siRNA molecule of at least 19 nucleotides, having at least one strand that is substantially complementary to at least ten but no more than thirty consecutive nucleotides of a nucleic acid that encodes a TNF-α, and that reduces the expression of the TNF-α protein.

The siRNA may also comprise an alteration of one or more nucleotides. Such alterations can include the addition of non-nucleotide material, such as to the end(s) of the 19 to 25 nucleotide RNA or internally (at one or more nucleotides of the RNA). In certain aspects, the RNA molecule contains a 3′-hydroxyl group. Nucleotides in the RNA molecules of the present invention can also comprise non-standard nucleotides, including non-naturally occurring nucleotides or deoxyribonucleotides. The double-stranded oligonucleotide may contain a modified backbone, for example, phosphorothioate, phosphorodithioate, or other modified backbones known in the art, or may contain non-natural internucleoside linkages. Additional modifications of siRNAs (e.g., 2′-O-methyl ribonucleotides, 2′-deoxy-2′-fluoro ribonucleotides, “universal base” nucleotides, 5-C-methyl nucleotides, one or more phosphorothioate internucleotide linkages, and inverted deoxyabasic residue incorporation) can be found in U.S. Application Publication 2004/0019001 and U.S. Pat. No. 6,673,611 (each of which is incorporated by reference in its entirety). Collectively, all such altered nucleic acids or RNAs described above are referred to as modified siRNAs.

(3) Messenger RNA (mRNA) Molecules

In further aspects, a polynucleotide of the embodiments is a mRNA molecule. For example, the mRNA may encode a therapeutic polypeptide or an antigen. In some aspects, mRNA molecules comprise a 5′ cap; a 5′ UTR; a 3′UTR; and/or a poly-A tail. mRNA molecules can provide a more direct method of expressing a polypeptide of interest in a target cell. However, such molecules are typically highly liable and rapidly degraded. However, in some aspects, LNP and/or URF processing according to the embodiments can be used to substantially stabilize mRNA. In prefer aspects, mRNA is provided encapsulated in or in complex with LNPs.

(4) Intact Cells

In some aspects, compositions of the embodiments comprise intact and/or living cells. For example, the cells can be eukaryotic, archaeal cells and/or bacterial cells. For example, the cells can comprise human cells (e.g., human iPS cells), fungal cells (e.g., yeast cell), or plant cells. In some aspects, the cells comprise bacterial cells. The bacterian may be gram positive or gram negative bacteria. For example, the cells may comprise bacteria that are protective to crop plants or express proteins that help control insect damage. In further aspects, the bacteria can be bacteria that are beneficial to human subject, such healthy gut bacteria. In some aspects, the cells are engineered cells, such as engineered bacteria.

In still further aspects, a bacterial composition of the embodiments can be a probiotic composition. For example, such a probiotic composition may comprise one or more bacteria from Bacteroidetes, Firmicutes, Proteobacteria, Verrucomicrobiae, and Actinobacteria. In some aspects, comprises one or more of a Actinobacteria, Bacteroidia, Bacilli, Clostridia, Erysipelotrichi, Alphaproteobacteria, Betaproteobacteria, Gammaproteobacteria, Mollicutes, and Verrucomicrobiae.

In still further aspects, a bacterial cell can be an attenuated or inactivated bacterial cell (e.g., for use in a vaccine). For example the attenuated or inactivated bacteria can be Streptococcus agalactiae, Legionella pneumophilia, Streptococcus pyogenes, Escherichia coli, Neisseria gonorrhosae, Neisseria meningitidis, Pneumococcus, Hemophilis influenzae B, Treponema pallidum, Lyme disease spirochetes, Pseudomonas aeruginosa, Mycobacterium leprae, Brucella abortus, Mycobacterium tuberculosis, Plasmodium falciparum, Plasmodium vivax, Toxoplasma gondii, Trypanosoma rangeli, Trypanosoma cruzi, Trypanosoma rhodesiensei, Trypanosoma brucei, Schistosoma mansoni, Schistosoma japanicum, Babesia bovis, Elmeria tenella, Onchocerca volvulus, Leishmania tropica, Trichinella spiralis, Theileria parva, Taenia hydatigena, Taenia ovis, Taenia saginata, Echinococcus granulosus, Mesocestoides corti, Mycoplasma arthritidis, M. hyorhinis, M. orale, M. arginini, Acholeplasma laidlawii, M. salivarium, M. pneumoniae, Candida albicans, Cryptococcus neoformans, Histoplasma capsulatum, Coccidioides immitis, Blastomyces dermatitidis, Aspergillus fumigatus, Penicillium marneffei, Bacillus anthracis, Bartonella, Bordetella pertussis, Brucella—all serotypes, Chlamydia trachomatis, Chlamydia pneumoniae, Clostridium botulinum—anything from clostridium serotypes, Haemophilus influenzae, Helicobacter pylori, Klebsiella—all serotypes, Legionella—all serotypes, Listeria, Mycobacterium—all serotypes, Mycoplasma—human and animal serotypes, Rickettsia—all serotypes, Shigella—all serotypes, Staphylococcus aureus, Streptococcus—S. pneumoniae, S. pyogenes, Vibrio cholera, Yersinia enterocolitica, and/or Yersinia pestis.

(5) Viruses

In still further aspects, compositions of the embodiments comprise viruses, viral vector and/or VLPs. For example, the virus can be a virus that infects mammalian cells or bacterial cells (a bacteriophage). In preferred aspects, the virus comprises a bacteriophage that infects bacteria that are pathogenic to human subjects. In still more preferred aspects, the bacteriophage infects bacteria that cause lung infections.

In still further aspects, a virus can be an attenuated or inactivated virus (e.g., for use in a vaccine). For example, the attenuated or inactivated virus can be from the family Arenaviridae (e.g., Lymphocytic choriomeningitis virus), Arterivirus (e.g., Equine arteritis virus), Astroviridae (Human astrovirus 1), Birnaviridae (e.g., Infectious pancreatic necrosis virus, Infectious bursal disease virus), Bunyaviridae (e.g., California encephalitis virus Group), Caliciviridae (e.g., Caliciviruses), Coronaviridae (e.g., Human coronaviruses 299E and OC43), Deltavirus (e.g., Hepatitis delta virus), Filoviridae (e.g., Marburg virus, Ebola virus), Flaviviridae (e.g., Yellow fever virus group, Hepatitis C virus), Hepadnaviridae (e.g., Hepatitis B virus), Herpes viridae (e.g., Epstein-Bar virus, Simplexvirus, Varicellovirus, Cytomegalovirus, Roseolovirus, Lymphocryptovirus, Rhadinovirus), Orthomyxoviridae (e.g., Influenzavirus A, B, and C), Papovaviridae (e.g., Papillomavirus), Paramyxoviridae (e.g., Paramyxovirus such as human parainfluenza virus 1, Morbillivirus such as Measles virus, Rubulavirus such as Mumps virus, Pneumovirus such as Human respiratory syncytial virus), Picornaviridae (e.g., Rhinovirus such as Human rhinovirus 1A, Hepatovirus such Human hepatitis A virus, Human poliovirus, Cardiovirus such as Encephalomyocarditis virus, Aphthovirus such as Foot-and-mouth disease virus 0, Coxsackie virus), Poxyiridae (e.g., Orthopoxvirus such as Variola virus or monkey poxvirus), Reoviridae (e.g., Rotavirus such as Groups A-F rotaviruses), Retroviridae (Primate lentivirus group such as human immunodeficiency virus 1 and 2), Rhabdoviridae (e.g., rabies virus), Togaviridae (e.g., Rubivirus such as Rubella virus), Human T-cell leukemia virus, Murine leukemia virus, Vesicular stomatitis virus, Wart virus, Blue tongue virus, Sendai virus, Feline leukemia virus, Simian virus 40, Mouse mammary tumor virus, Dengue virus, HIV-1 and HIV-2, West Nile, H1N1, SARS, 1918 Influenza, Tick-borne encephalitis virus complex (Absettarov, Hanzalova, Hypr), Russian Spring-Summer encephalitis virus, Congo-Crimean Hemorrhagic Fever virus, Junin Virus, Kumlinge Virus, Marburg Virus, Machupo Virus, Kyasanur Forest Disease Virus, Lassa Virus, Omsk Hemorrhagic Fever Virus, FIV, SIV, Herpes simplex 1 and 2, Herpes Zoster, Human parvovirus (B19), Respiratory syncytial virus, Pox viruses (all types and serotypes), Coltivirus, Reoviruses—all types, and/or Rubivirus (rubella).

In yet further aspects, the virus can be viral vector, such as an engineered viral vector. Such viral vectors in include, but are not limited to adenoviral vectors, retroviral vectors and adeno-associated viral vectors.

B. Nanoparticle and Nanoparticle Complexes

As used herein, the term “nanoparticle” refers to any material having dimensions in the 1-1,000 nm range. In some embodiments, nanoparticles have dimensions in the 50-500 nm range. Nanoparticles used in the present embodiments include such nanoscale materials as a lipid-based nanoparticle, a superparamagnetic nanoparticle, a nanoshell, a semiconductor nanocrystal, a quantum dot, a polymer-based nanoparticle, a silicon-based nanoparticle, a silica-based nanoparticle, a metal-based nanoparticle, a fullerene and a nanotube (Ferrari, 2005). The conjugation of polypeptide or nucleic acids to nanoparticles provides structures with potential application for targeted delivery, controlled release, enhanced cellular uptake and intracellular trafficking, and molecular imaging of therapeutic peptides in vitro and in vivo (West, 2004; Stayton et al., 2000; Ballou et al., 2004; Frangioni, 2003; Dubertret et al., 2002; Michalet et al., 2005; Dwarakanath et al., 2004.

(1) Chitosan Nanoparticles

In some aspects, nanoparticles for use in accordance with the embodiments include chitosan as a component. Generally, chitosans are a family of cationic, binary hetero-polysaccharides composed of (1→4)-linked 2-acetamido-2-deoxy-β-D-glucose (GlcNAc, A-unit) and 2-amino-2-deoxy-β-D-glucose, (GlcN; D-unit) (Varum et al., 1991). The chitosan has a positive charge, stemming from the de-acetylated amino group (—NH₃⁺). Chitosan, chitosan derivatives, or salts (e.g., nitrate, phosphate, sulphate, hydrochloride, glutamate, lactate or acetate salts) of chitosan may be used and are included within the meaning of the term “chitosan.” As used herein, the term “chitosan derivatives” is intended to include ester, ether, or other derivatives formed by bonding of acyl and/or alkyl groups with —OH groups, but not the NH₂ groups, of chitosan. Examples are O-alkyl ethers of chitosan and O-acyl esters of chitosan. Modified chitosans, particularly those conjugated to polyethylene glycol, are also considered “chitosan derivatives.” Many chitosans and their salts and derivatives are commercially available (e.g., SigmaAldrich, Milwaukee, Wis.). In preferred aspects, chitosan nanoparticles of the embodiments are PEGylated.

Methods of preparing chitosans and their derivatives and salts are also known, such as boiling chitin in concentrated alkali (50% w/v) for several hours. This produces chitosan wherein 70%-75% of the N-acetyl groups have been removed. A non-limiting example of a chitosan, wherein all of the N-acetyl groups have been removed, is shown below:

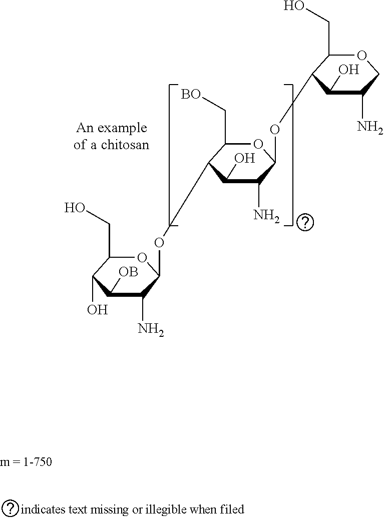

Chitosans may be obtained from any source known to those of ordinary skill in the art. For example, chitosans may be obtained from commercial sources. Chitosans may be obtained from chitin, the second most abundant biopolymer in nature. Chitosan is prepared by N-deacetylation of chitin. Chitosan is commercially available in a wide variety of molecular weight (e.g., 10-1000 kDa) and usually has a degree of deacetylation ranging between 70%-90%.

The chitosan (or chitosan derivative or salt) used preferably has a molecular weight of 4,000 Dalton or more, preferably in the range 25,000 to 2,000,000 Dalton, and most preferably about 50,000 to 300,000 Dalton. Chitosans of different molecular weights can be prepared by enzymatic degradation of high molecular weight chitosan using chitosanase or by the addition of nitrous acid. Both procedures are well known to those skilled in the art and are described in various publications (Li et al., 1995; Allan and Peyron, 1995; Domard and Cartier, 1989). The chitosan is water-soluble and may be produced from chitin by deacetylation to a degree of greater than 40%, preferably between 50% and 98%, and more preferably between 70% and 90%.

Some methods of producing chitosan involve recovery from microbial biomass, such as the methods taught by U.S. Pat. No. 4,806,474 and U.S. Patent Application No. 2005/0042735, herein incorporated by reference. Another method, taught by U.S. Pat. No. 4,282,351, teaches only how to create a chitosan-beta-glucan complex.

The chitosan, chitosan derivative, or salt used in the present invention is water soluble. Chitosan glutamate is water soluble. By “water soluble” it is meant that that the chitosan, chitosan derivative, or salt dissolves in water at an amount of at least 10 mg/ml at room temperature and atmospheric pressure. The chitosan, chitosan derivative, or salt used in the present invention has a positive charge.

Additional information regarding chitosan and chitosan derivatives can be found in U.S. Patent App. Pub. Nos. 2007/0167400, 2007/0116767, 2007/0311468, 2006/0277632, 2006/0189573, 2006/0094666, 2005/0245482, 2005/0226938, 2004/0247632, and 2003/0129730, each of which is herein specifically incorporated by reference.

In preferred aspects, Chitosan nanoparticles of the embodiments are provided in complex with a nucleic acid, such as DNA.

(2) Lipid Nanoparticles (LNPs)

Lipid-based nanoparticles include liposomes, lipid preparations and lipid-based vesicles (e.g., DOTAP:cholesterol vesicles). Lipid-based nanoparticles may be positively charged, negatively charged or neutral. In certain embodiments, the lipid-based nanoparticle is neutrally charged (e.g., a DOPC liposome).

A “liposome” is a generic term encompassing a variety of single and multilamellar lipid vehicles formed by the generation of enclosed lipid bilayers or aggregates. Liposomes may be characterized as having vesicular structures with a bilayer membrane, generally comprising a phospholipid, and an inner medium that generally comprises an aqueous composition. Liposomes provided herein include unilamellar liposomes, multilamellar liposomes and multivesicular liposomes. Liposomes provided herein may be positively charged, negatively charged or neutrally charged. In certain embodiments, the liposomes are neutral in charge.

A multilamellar liposome has multiple lipid layers separated by aqueous medium. They form spontaneously when lipids comprising phospholipids are suspended in an excess of aqueous solution. The lipid components undergo self-rearrangement before the formation of closed structures and entrap water and dissolved solutes between the lipid bilayers (Ghosh and Bachhawat, 1991). Lipophilic molecules or molecules with lipophilic regions may also dissolve in or associate with the lipid bilayer.

In specific aspects, a polypeptide or nucleic acids may be, for example, encapsulated in the aqueous interior of a liposome, interspersed within the lipid bilayer of a liposome, attached to a liposome via a linking molecule that is associated with both the liposome and the polypeptide/nucleic acid, entrapped in a liposome, complexed with a liposome, or the like.

Additional liposomes which may be useful with the present embodiments include cationic liposomes, for example, as described in WO02/100435A1, U.S. Pat. No. 5,962,016, U.S. Application 2004/0208921, WO03/015757A1, WO04029213A2, U.S. Pat. Nos. 5,030,453, and 6,680,068, all of which are hereby incorporated by reference in their entirety without disclaimer. A process of making liposomes is also described in WO04/002453A1. Neutral lipids can be incorporated into cationic liposomes (e.g., Farhood et al., 1995). Various neutral liposomes which may be used in certain embodiments are disclosed in U.S. Pat. No. 5,855,911, which is incorporated herein by reference. These methods differ in their respective abilities to entrap aqueous material and their respective aqueous space-to-lipid ratios.

The size of a liposome varies depending on the method of synthesis. Liposomes in the present embodiments can be a variety of sizes. In certain embodiments, the liposomes are small, e.g., less than about 100 nm, about 90 nm, about 80 nm, about 70 nm, about 60 nm, or less than about 50 nm in external diameter. For example, in general, prior to the incorporation of nucleic acid, a DOTAP:cholesterol liposome for use according to the present embodiments comprises a size of about 50 to 500 nm. Such liposome formulations may also be defined by particle charge (zeta potential) and/or optical density (OD). For instance, a DOTAP:cholesterol liposome formulation will typically comprise an OD₄₀₀ of less than 0.45 prior to nucleic acid incorporation. Likewise, the overall charge of such particles in solution can be defined by a zeta potential of about 50-80 mV.

In preparing such liposomes, any protocol described herein, or as would be known to one of ordinary skill in the art may be used. Additional non-limiting examples of preparing liposomes are described in U.S. Pat. Nos. 4,728,578, 4,728,575, 4,737,323, 4,533,254, 4,162,282, 4,310,505, and 4,921,706; International Applications PCT/US85/01161 and PCT/US89/05040; U.K. Patent Application GB 2193095 A; Mayer et al., 1986; Hope et al., 1985; Mayhew et al. 1987; Mayhew et al., 1984; Cheng et al., 1987; and Liposome Technology, 1984, each incorporated herein by reference).

In certain embodiments, the lipid based nanoparticle is a neutral liposome (e.g., a DOPC liposome). “Neutral liposomes” or “non-charged liposomes”, as used herein, are defined as liposomes having one or more lipid components that yield an essentially-neutral, net charge (substantially non-charged). By “essentially neutral” or “essentially non-charged”, it is meant that few, if any, lipid components within a given population (e.g., a population of liposomes) include a charge that is not canceled by an opposite charge of another component (i.e., fewer than 10% of components include a non-canceled charge, more preferably fewer than 5%, and most preferably fewer than 1%). In certain embodiments, neutral liposomes may include mostly lipids and/or phospholipids that are themselves neutral under physiological conditions (i.e., at about pH 7).

Liposomes and/or lipid-based nanoparticles of the present embodiments may comprise a phospholipid. In certain embodiments, a single kind of phospholipid may be used in the creation of liposomes (e.g., a neutral phospholipid, such as DOPC, may be used to generate neutral liposomes). In other embodiments, more than one kind of phospholipid may be used to create liposomes.

Phospholipids include, for example, phosphatidylcholines, phosphatidylglycerols, and phosphatidylethanolamines; because phosphatidylethanolamines and phosphatidyl cholines are non-charged under physiological conditions (i.e., at about pH 7), these compounds may be particularly useful for generating neutral liposomes. In certain embodiments, the phospholipid DOPC is used to produce non-charged liposomes. In certain embodiments, a lipid that is not a phospholipid (e.g., a cholesterol) may be used

Phospholipids include glycerophospholipids and certain sphingolipids. Phospholipids include, but are not limited to, dioleoylphosphatidylycholine (“DOPC”), egg phosphatidylcholine (“EPC”), dilauryloylphosphatidylcholine (“DLPC”), dimyristoylphosphatidylcholine (“DMPC”), dipalmitoylphosphatidylcholine (“DPPC”), distearoylphosphatidylcholine (“DSPC”), 1-myristoyl-2-palmitoyl phosphatidylcholine (“MPPC”), 1-palmitoyl-2-myristoyl phosphatidylcholine (“PMPC”), 1-palmitoyl-2-stearoyl phosphatidylcholine (“PSPC”), 1-stearoyl-2-palmitoyl phosphatidylcholine (“SPPC”), dilauryloylphosphatidylglycerol (“DLPG”), dimyristoylphosphatidylglycerol (“DMPG”), dipalmitoylphosphatidylglycerol (“DPPG”), distearoylphosphatidylglycerol (“DSPG”), distearoyl sphingomyelin (“DS SP”), distearoylphophatidylethanolamine (“DSPE”), dioleoylphosphatidylglycerol (“DOPG”), dimyristoyl phosphatidic acid (“DMPA”), dipalmitoyl phosphatidic acid (“DPPA”), dimyristoyl phosphatidylethanolamine (“DMPE”), dipalmitoyl phosphatidylethanolamine (“DPPE”), dimyristoyl phosphatidylserine (“DMPS”), dipalmitoyl phosphatidylserine (“DPPS”), brain phosphatidylserine (“BPS”), brain sphingomyelin (“BSP”), dipalmitoyl sphingomyelin (“DPSP”), dimyristyl phosphatidylcholine (“DMPC”), 1,2-distearoyl-sn-glycero-3-phosphocholine (“DAPC”), 1,2-diarachidoyl-sn-glycero-3-phosphocholine (“DBPC”), 1,2-dieicosenoyl-sn-glycero-3-phosphocholine (“DEPC”), dioleoylphosphatidylethanolamine (“DOPE”), palmitoyloeoyl phosphatidylcholine (“POPC”), palmitoyloeoyl phosphatidylethanolamine (“POPE”), lysophosphatidylcholine, lysophosphatidylethanolamine, and dilinoleoylphosphatidylcholine.

Phospholipids may be from natural or synthetic sources. However, phospholipids from natural sources, such as egg or soybean phosphatidylcholine, brain phosphatidic acid, brain or plant phosphatidylinositol, heart cardiolipin and plant or bacterial phosphatidylethanolamine are not used, in certain embodiments, as the primary phosphatide (i.e., constituting 50% or more of the total phosphatide composition) because this may result in instability and leakiness of the resulting liposomes.

C. Excipients

In some aspects, the present disclosure comprises one or more excipients formulated into pharmaceutical compositions. In some embodiments, the excipients used herein are water soluble excipients. These water soluble excipients include saccharides such as disaccharides. In some cases, the excipient comprises sucrose, trehalose, or lactose, a trisaccharide such as fructose, sucrose, glucose, glacatose, or raffinose, polysaccharides such as starches or cellulose, or a sugar alcohol such as xylitol, sorbitol, or mannitol. In some embodiments, these excipients are solid at room temperature. Some non-limiting examples of sugar alcohols include erythritol, threitol, arabitol, xylitol, ribitol, mannitol, sorbitol, galactitol, fucitol, iditol, inositol, volemitol, isomalt, maltitol, lactitol, maltotritol, maltotetraitol, or a polyglycitol. In some aspects, the present pharmaceutical compositions may further exclude a hydrophobic or waxy excipient such as waxes and oils. Some non-limiting examples of hydrophobic excipients include hydrogenated oils and partially hydrogenated oils, palm oil, soybean oil, castor oil, carnauba wax, beeswax, palm wax, white wax, castor wax, or lanoline. Additionally, the present disclosure may further comprise one or more amino acids or an amide or ester derivative thereof. In some embodiments, the amino acids used may be one of the 20 canonical amino acids such as glycine, alanine, valine, isoleucine, leucine, methionine, phenylalanine, tyrosine, tryptophan, serine, threonine, asparagine, glutamine, cysteine, selenocysteine, proline, arginine, histidine, lysine, aspartic acid, or glutamic acid. These amino acids may be in the D or L orientation or the amino acids may be an α-, β-, γ-, or δ-amino acids. In other embodiments, one of the common non-canonical amino acids may be used such as carnitine, GABA, carboxyglutamic acid, levothyroxine, hydroxyproline, seleonmethionine, beta alanine, ornithine, citrulline, dehydroalanine, δ-aminolevulinic acid, or 2-aminoisobutyric acid.

In some aspects, the amount of the excipient in the precursor solution for making a powder composition is from about 0.5% to about 20% w/w, from about 1% to about 10% w/w, from about 2% to about 8% w/w, or from about 2% to about 5% w/w. The amount of the excipient in the precursor solution comprises from about 0.5%, 0.75%, 1%, 1.25%, 1.5%, 1.75%, 2%, 2.5%, 3%, 3.5%, 4%, 4.5%, 5%, 6%, 7%, 8%, 9%, to about 10% w/w, or any range derivable therein. In one embodiment, the amount of the excipient in a dry powder of the embodiments is about 10% to 99.5% w/w of the total weight of the pharmaceutical composition, such as about 50% to 99%, 75% to 99% or 80% to 98%.

III. Definitions

The use of the word “a” or “an” when used in conjunction with the term “comprising” in the claims and/or the specification may mean “one,” but it is also consistent with the meaning of “one or more,” “at least one,” and “one or more than one.” As used herein “another” may mean at least a second or more.

As used herein, the terms “drug”, “pharmaceutical”, “therapeutic agent”, and “therapeutically active agent” are used interchangeably to represent a compound which invokes a therapeutic or pharmacological effect in a human or animal and is used to treat a disease, disorder, or other condition. In some embodiments, these compounds have undergone and received regulatory approval for administration to a living creature.

The use of the term “or” in the claims is used to mean “and/or” unless explicitly indicated to refer to alternatives only or the alternatives are mutually exclusive. As used herein “another” may mean at least a second or more.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used in this specification, the term “significant” (and any form of significant such as “significantly”) is not meant to imply statistical differences between two values but only to imply importance or the scope of difference of the parameter.

Throughout this application, the term “about” is used to indicate that a value includes the inherent variation of error for the device, the method being employed to determine the value, or the variation that exists among the study subjects or experimental studies. Unless another definition is applicable, the term “about” refers to ±10% of the indicated value.

As used herein, the term “substantially free of” or “substantially free” in terms of a specified component, is used herein to mean that none of the specified component has been purposefully formulated into a composition and/or is present only as a contaminant or in trace amounts. The total amount of all containments, by-products, and other material is present in that composition in an amount less than 2%. The term “more substantially free of” or “more substantially free” is used to represent that the composition contains less than 1% of the specific component. The term “essentially free of” or “essentially free” contains less than 0.5% of the specific component.

As used herein, the term “nanoparticle” has its customary and ordinary definition and refers to discrete particles which behave as a whole unit rather than as individual molecules within the particle. A nanoparticle may have a size from about 1 to about 10,000 nm with ultrafine nanoparticles having a size from 1 nm to 100 nm, fine particles having a size from 100 nm to 2,500 nm, and coarse particles having a size from 2,500 nm to 10,000 nm. In some embodiments, the nanoaggregates described herein may comprise a composition of multiple nanoparticles and have a size from about 10 nm to about 100 μm.

Notwithstanding that the numerical ranges and parameters setting forth the broad scope of the invention are approximations, the numerical values set forth in the specific examples are reported as precisely as possible. Any numerical value, however, inherently contain certain errors necessarily resulting from the standard deviation found in their respective testing measurements and parameters.

### IV. EXAMPLES

To facilitate a better understanding of the present disclosure, the following examples of specific embodiments are given. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure. In no way should the following examples be read to limit or define the entire scope of the disclosure.

### Example 1—Inhalable Bacteriophage Solid Formulations Using Thin Film Freezing Technology

### A. Material and Methods

1. Materials

D-(+)-trehalose, dihydrate, sodium chloride, magnesium sulfate, sucrose, and Lysogeny broth (LB) media, LB agar, were purchased from Thermo Fisher Scientific (Waltham, Mass., US); leucine and mannitol were purchased from Spectrum (New Brunswick, N.J., US); T7 bacteriophage and its host BL21 bacteria strain were purchased from Millipore Sigma (Burlington, Mass., US); Phosphate saline buffer (PBS), Trizma® base, Tris-HCl were purchased from Sigma-Aldrich (St. Louis, Mo., US).

2. Methods

T7 amplification and phage reconstitution. T7 phage were amplified according to manufacturer's protocol. Briefly, phage were added to BL21 liquid cultures (0D600 of 0.2-0.3) at a multiplicity of infection (MOI) 0.001-0.01 and amplified for 1-3 hours at 37° C., 250 RPM until lysis was observed. Bacterial lysate was collected, clarified with 5M NaCl/LB and spun down at 10,000 rpm in a Sorvall XFR Centrifuge (Thermo Fisher Scientific, Waltham, Mass., US) for 30 minutes at 4° C. The supernatant containing the phage was collected and phage were further precipitated by incubating phage samples with a 50% PEG 8000 solution overnight at 4° C. Once precipitated, phage were pelleted by spinning down at 14,000 rpm and resuspended in either PBS or SM buffer and collected in 1.5 mL microcentrifuge tubes. To further purify the phage, a second PEG precipitation step was performed with the resuspended phage, by precipitating with 50% PEG 8000 solution on ice for at least 30 minutes. This lysate-PEG mixture was then centrifuged at 14,000 rpm for 30 minutes and the resulting phage pellet was resuspended in 50-100 μL of either PBS or SM buffer. Amplified phage was quantified by standard double-layer plaque assay and stored at 4° C.

Phage viability test. The amount of viable phage in the solution and powder samples was determined by titering (i.e. an activity counting assay). The TFFD processed phage powders were reconstituted in sterile water to a final concentration of 10 mg/ml. In the viability test for freezing step, the frozen thin films were collected and thaw at room temperature before titering. The lytic bioactivity of phage was assayed by performing a standard double-layer plaque assay. Briefly, testing phage solution were prepared in 10-fold serial dilutions using LB media. Ten microliters of each dilution was added to 200 μL of BL21 bacteria (0D600=1.0) and 1 mL of melted LB top agar. After briefly vortexed, the mixture was plated onto pre-warmed 6-well plates that has 5 mL solidified agar bottom. Plates were incubated at 37° C. for approximately 3-4 hours, until plaques were visible for counting and quantification. Titer loss was calculated by dividing the titer of initial formulation solution by the titer of each sample.

Formulation preparation. Several excipients that were commonly used in solid phage formulation research were selected, including three disaccharides (lactose, sucrose, and trehalose), one sugar alcohol (mannitol), and one amino acid (leucine). These excipients were incorporated in the formulations either alone or combined with another one to form binary excipient matrix. The combination was sugar and mannitol or sugar and leucine in a ratio of 90:10 to 50:50. The formulation solutions were prepared in a solid content range of 0.25% to 10% which corresponds to the solution concentrations of 2.5 mg/mL to 100 mg/mL. Solid content refers to the weight to volume concentration of all components in the pre-TFFD solution formulation. The initial titers of phage stocks were in multitude of 10¹¹ PFU/mL to 10¹² PFU/mL and they were added to the formulations at 100 to 1000 folds dilution to achieve a final titer of 5×10⁸ to 10⁹ PFUl/mL, unless otherwise noted. The solutions were prepared in PBS (pH 7.4), SM buffer (pH 7.4-7.5), or water. SM buffer (without gelatin) was prepared according to the recipe provided by Cold Spring Harbor Protocol.

Manufacturing phage powder by TFFD. Aqueous phage solutions were passed through a standard 5 mL or 10 mL syringe. The droplets fell from a height of 10 cm above an absolute-flat bottom stainless-steel container which was pre-chilled by submerging it to liquid nitrogen. As a result of thermal conductivity through the steel, the resulting equilibrium surface temperatures of surface of the container's bottom were below freezing point of the solutions and could go down to as low as colder than −100° C. In this experiment, the working temperature was controlled by adjusting the height of the container in the liquid nitrogen. The temperature was controlled within −65 to −75° C. unless otherwise noted. Before and during the runs, the surface temperature of the container's bottom was verified with a thermocouple that was installed on the bottom surface with a wire. Upon touching the surface of the bottom of the stainless-steel container, droplets deformed into thin films and froze immediately. The frozen thin films were manually removed from the surface by a stainless-steel blade. The container with frozen thin films was then filled with liquid nitrogen. The films and liquid nitrogen were poured into a 20 mL lyophilization vial which was then covered with a double layer Kim-wipe to prevent particles from exiting the vial during vacuum drying. Finally, the vials were transferred directly to a −80° C. freezer to evaporate excess liquid nitrogen and hold till being placed into lyophilizer.

A Virtis Advantage Lyophilizer (The Virtis Company, Inc., Gardiner, N.Y.) was used to dry the frozen slurries. Primary drying was carried out at −40° C. for 2000 min at 100 mTorr and secondary drying at 25° C. for 1250 min at 100 mTorr. A 12-h linear ramp of the shelf temperature from −40° C. to +25° C. was used at 100 mTorr between these two drying steps. After the cycle was done, the containers were capped tightly and then stored in a vacuum chamber immediately after being removed from the lyophilizer.

Geometric particle size measurement. GPSD of TFFD processed phage powders was analyzed using a Sympatec HELOS laser diffraction instrument (Sympatec GmbH, Germany) equipped with RODOS dispersion. Measurements were taken every 10 ms following powder dispersion at 3 bar. Measurements that are between 5% and 25% optical density were then averaged to determine the particle size distribution. The particle sizes by volume were reported at the 10, 50, and 90 percentiles (e.g. Dv10/50/90), respectively, as well as the percentages of particles falling into 1-5 μm size range. Span was calculated with following equation: Span=(Dv90-Dv10)/Dv50.

Images by scanning electronic microscope (SEM). The morphology of TFFD processed phage powders was analyzed with Zeiss Supra 40VP SEM (Carl Zeiss Microscopy GmbH, Jena, Germany). Samples were mounted on aluminum SEM stubs using a carbon conductive tape and were coated with 15 nm of platinum/palladium (Pt/Pd) using a Cressington sputter coater 208 HR (Cressington Scientific Instruments Ltd., Watford, UK).

X-ray diffraction (XRD) pattern. The crystallinity of TFFD processed phage powder was detected using an X-ray diffractometer (MiniFlex 600, Rigaku Co., Japan) under ambient conditions. Powders were spread on the glass slides and were exposed to Cu Kα radiation at 15 mA and 40 kV. The scattered intensity was collected by a detector for a 20 ranging from 5 to 50° at a step size of 0.025°, and a speed of 2°/min, respectively.

Thermogravimetric analysis (TGA). Thermogravimetric analysis was conducted using the Mettler Thermogravimetric Analyzer (Mettler Toledo, Columbus, Ohio, US). Samples in a size of 1-3 mg were loaded in 70 μl alumina pans and the pans were loosely capped with a lid that has a vent hole. Samples were heated up from 35° C. to 400° C. at a rate of 10° C./min. The system was purged by nitrogen at a flow rate of 50 L/min. The percentage of change in mass over initial mass was calculated and plotted against temperature. The percent of weight loss at 120° C. was used to determine the water content in powders.

Images by transmission electronic microscope (TEM). Selected sample powder was reconstituted with sterile water to a concentration of 10¹⁰ PFU/mL. A volume of 5 μL testing solution was gently dropped on the surface of a carbon-coated copper grid (CFT300-CU, Electron Microscopy Science, Hatfield, Pa., USA) and the liquid residue was taken up by a filter paper with capillary action. The grid was then stained with 5 μl of 2% uranyl acetate negative staining solution (pH=4.3) to improve visualization of the phage. Phage were imaged using a FEI Tecnai TEM (FEI Tecnai, OR, US) at 80 kV equipped with an AMT Advantage HR 1k×1k digital camera (Advanced Microscopy Techniques, MA, US).

Excipient Screening.

The three sugars, lactose, trehalose, and sucrose, were formulated with/without mannitol or leucine at different ratios in this study. The formulations in this study were processed at −70±5° C. and the solid content was 1% (w/v).

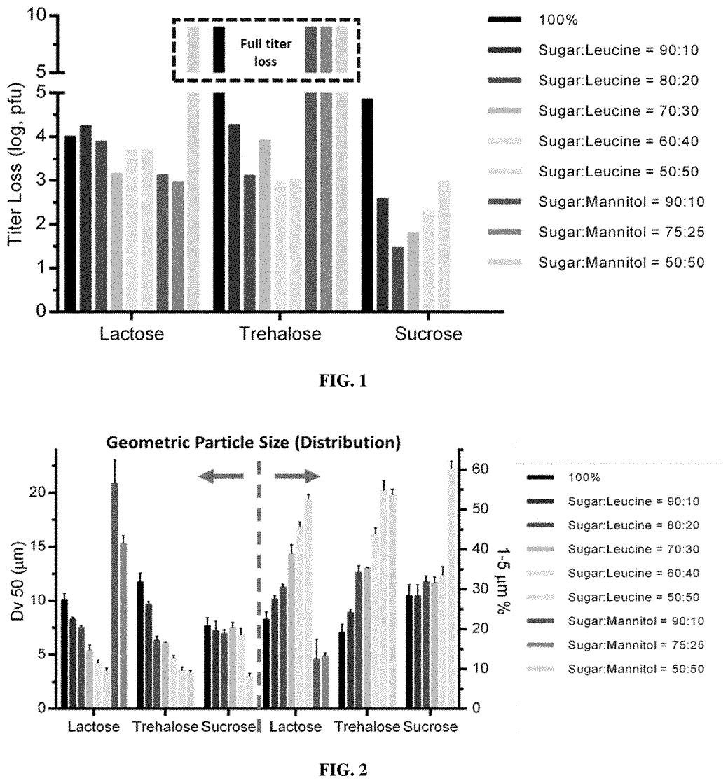

The titer loss results in FIG. 1 show that in general, sucrose containing formulations preserved the phage lytic activity better than lactose and trehalose. In addition, sugars alone could not sufficiently protect phage and has adverse effect on phage stability. Most of the mannitol containing formulations experienced full titer loss. It was obvious that mannitol was detriment to the phage. The negative impact of mannitol to phage was previously reported with lyophilized M13 phage research, in which it was observed that the titer loss increases with the increase of mannitol ratio in the mannitol-trehalose binary system. Among all formulations, sucrose:leucine 80:20, with a titer loss of 1.47 (log, PFU), was found to be the best formulation to preserve phage viability.

The effect of excipient on the pattern of GPSD was significant (FIG. 2). It was clearly demonstrated that with the increase of ratio of leucine in the excipient matrix, the particle sizes and percentile of 1-5 μm particles decreased and increased respectively. Interestingly, this trend was more obvious in lactose and trehalose groups than in sucrose group. Surprisingly, the addition of mannitol enlarged the particle sizes of lactose-mannitol samples. The Dv50 of sucrose: leucine 80:20 was 6.94±0.38 μm.

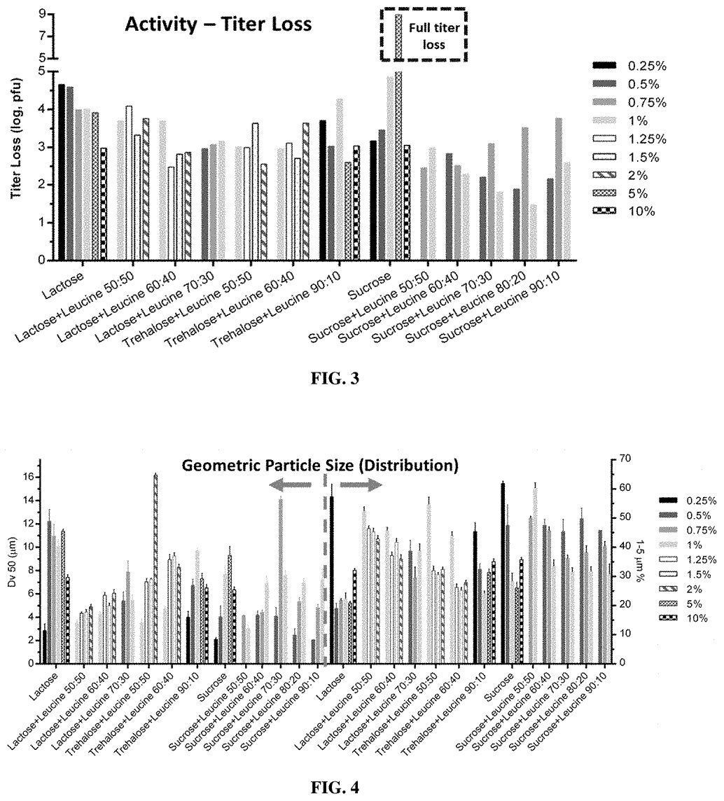

Solid content screening. Multiple excipient matrices were formulated in various levels of solid contents in formulations. The formulations in this study were processed at −70±5° C. The impact of solid content on the titer loss has no pattern although a weak of trend was observed that the titer loss was rise with more solid content in the formulation (FIG. 3). It is worth mentioning that in most of excipient matrix the powder collapsed after lyophilization when the solid content was 0.25%. Therefore, the solid content must be greater than 0.5% in formulations.

FIG. 4 shows the change of particle size distributions with the increase of solid contents in formulations. In general, particle size and solid content has a negative correlation, i.e., lower solid content generates smaller particle size. However, exceptions were seen in the tested formulation groups, for example, in lactose group the greatest particle size was when solid content was 0.5% instead of 10%.

Process temperature screening. Multiple excipient matrices were formulated in various levels of solid contents in formulations. The formulations in this study were processed at −70±5° C. and the solid content was 1% (w/v). Since excipient matrices sucrose: leucine 70:30 and 80:3 were found to be most effective in preserving the phage activity, they were used as the model formulations to explore the effect of freezing temperature on the titer and particle size of phage powders. As described in Method section, the temperature of the stainless-steel container's surface was changed by adjusting the level of liquid nitrogen contacted with the container. Temperatures were controlled to −40±5° C., −70±5° C., −100±5° C., and −120±5° C.

As the titer loss change showed in FIG. 5 indicated, the coldness has a affects phage viability negatively, meaning the lower the temperature, the greater the titer loss. Yet, the effect of temperature is limited as the difference between the highest and lowest titers were less than 0.5 (log, PFU). In addition, the trend and degree of impact can vary in different formulations.

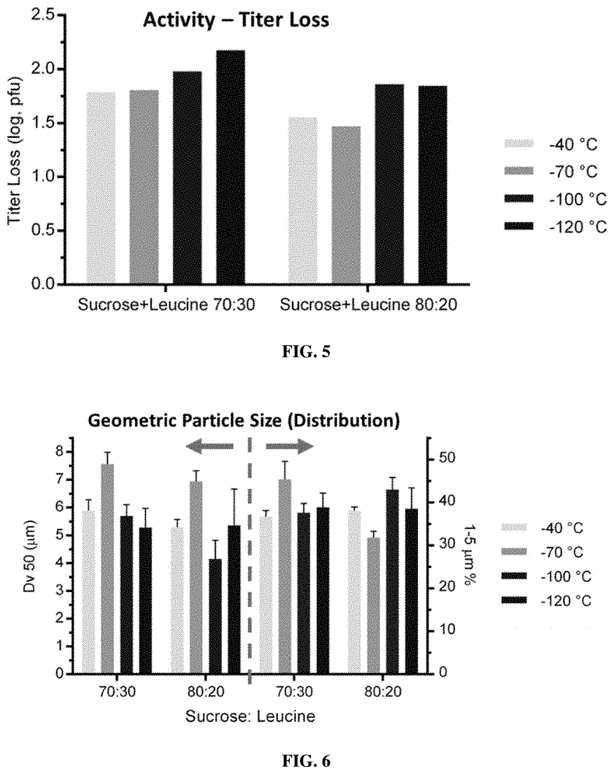

The effect of processing temperature on the particle size distribution of powders was irregular (see FIG. 6). This might be cause by the fact that the dimensions (size and thickness) of the disks (thin films) formed in different temperatures were different. With the decrease of the temperature, the disks become thicker and rounder. It was observed that the dropped liquid formed droplet shape and bounced around the stainless surface when the temperature went to lower than −125° C. In addition, disks attached tightly on the surface when process temperature went up to greater than −40° C. It is hypothesized that these observations were caused by Leidenfrost effect where a gas layer formed when the surface temperature of the stainless-steel is lower than a certain degree. The degree of the impact of gas layer highly depends on the temperature difference between the container's surface and the formulation droplets. The results became more complicated when disks' dimensions confounded other factors such as composition in the formulation because the surface tension varies with different formulation matrix.

Initial titer in formulations. The impact of initial titer of phage in the formulations were investigated by diluting the phage stock which was stored in PBS at an initial titer of 5×10¹¹ PFU/mL (also expressed as 5E11). The solid content was 0.5% (w/v) and the excipients were sucrose: leucine 80:20 formulations. The TFFD was conducted at −70±5° C.

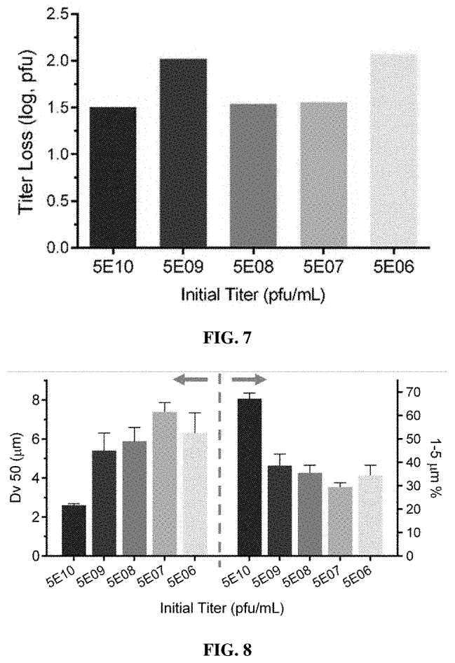

As FIG. 7 shows, the titer losses of 5×10¹⁰ PFU/mL, 5×10⁸ PFU/mL, and 5×10⁷ PFU/mL (also expressed as 5E10, 5E8, and 5E7) were in the similar level, approximately 1.50-1.55 (log, PFU) while the other initial titer levels lost 2.02 (in 5×10⁹ PFU/mL formulation) and 2.07 (in 5×10⁶ PFU/mL formulation), respectively.

The particle size was significantly impacted by the amount of phage in the formulations. The Dv50 of phage powders increased when the initial titer was reduced from 5E10 PFU/mL to 5E07 PFU/mL. The drastic change of particle sizes between 5E10 PFU/mL and 5E09 PFU/mL was likely due to the presence of residual salt molecules from the stock solution. The stock was diluted only 10 folds in 5E10 PFU/mL formulation, which can be sufficiently impactful to the crystallization behavior of the formulation during the process. It is encouraging to find out that the Dv50 was reduced to 2.61±0.07 μm and the percentile of 1-5 μm particles was improved to 67.2±2.42% (FIG. 8)

Impact of buffer system on different binary excipient matrices. Based on the previous findings that salt molecules in buffer system could affect particle size and potentially phage viability, a study was conducted to investigate this impact. PBS and SM buffer (without gelatin) has been chosen since they are both routinely used to store phages. The solid content of the formulations was 0.5% (w/v) and the TFF was conducted at −70±5° C.

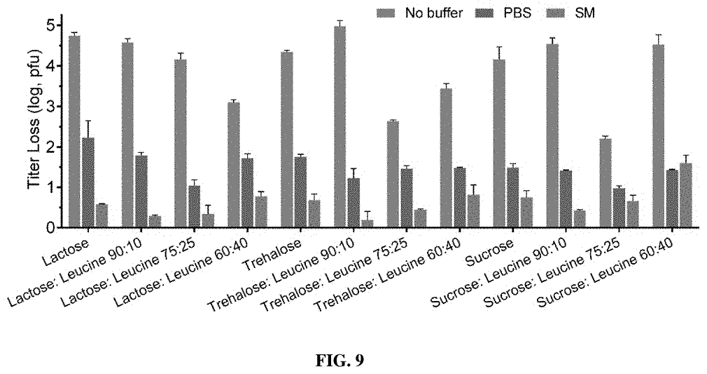

It is clearly demonstrated in FIG. 9 that the presence of buffer system has significant preservative effect on phage titers. Moreover, the levels of the impact were different between the two tested buffer systems. Generally, SM buffer samples lost less phage viability than PBS buffer samples. Among the excipient matrices, trehalose:leucine 90:10 has both the highest titer loss (no buffer sample, 4.97±0.14 log titer loss) and the lowest titer loss (SM buffer sample, 0.19±0.21 log titer loss).

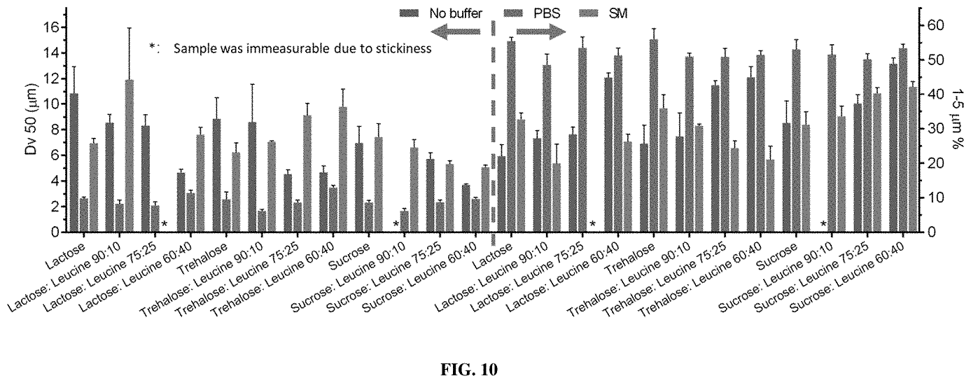

The Dv50 of the PBS containing powders was generally smaller than its no-buffer and SM buffer counterparts within each excipient matrix group. In contrast, the measurement results of SM buffer samples were significantly higher (FIG. 10). This is probably due to the fact that SM powders became very sticky when exposed to the ambient atmosphere for a certain amount of time. The similar phenomenon was observed in some of the no buffer samples but never occurred to PBS samples. The stickiness might have resulted from the high hygroscopicity of the powders. The local humidity at the testing time was 75% (data from weather.com).

Based on the results in FIG. 9 and FIG. 10, two formulation groups were selected to be investigated in the further studies: trehalose:leucine 90:10 and sucrose: leucine 75:25. Even though preserving phage viability well, sucrose: leucine 90:10 and lactose:leucine 90:10/75:25 were not chosen since the particle size of SM buffer samples in these two groups were either too high or immeasurable as a result of the stickiness.

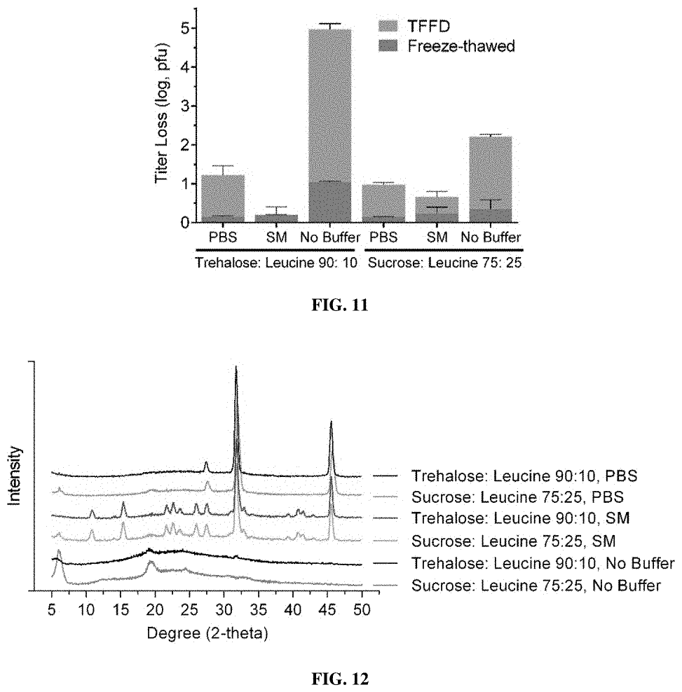

Titer loss in different process steps. TFFD involves two steps that could impair phage viabilities: freezing and drying. In order to learn the extent of activity loss in each step, titers were examined after freezing and drying, respectively. As shown in FIG. 11, incorporating buffer system reduced titer loss in both freezing and drying steps regardless of excipient compositions. Phage survived the most in PBS buffer system during the freezing step. Most titer loss occurred in drying step in PBS and no buffer samples. In contrast, no titer loss was found in the drying step in SM buffer sample when the other excipients were trehalose:leucine 90:10.

Buffer system are routinely included in solid products due to their ability to stabilize the pH during freezing process. However, this might not be the protection mechanism in this case since phage are generally insensitive to pH and pH shift can be limited in a rapid freezing process. Therefore, the protection might be a result of molecular-level interactions between phage capsid proteins and salt molecules. The protective effect of buffer system in drying step could be indirectly: the existence of salt molecules changed the crystal shapes in the frozen thin films, which ultimately lead to different drying behavior.

Geometric particle size distribution. The geometric particle size was measured using laser diffractometry (data in Table 1). The particles were generally smaller in sucrose: leucine 75:25 group than in trehalose:leucine 90:10 group. This can be attributed to the 15% more leucine in sucrose: leucine 75:25 formulation system. The addition of buffer systems generally decreased particle sizes of phage powders. Both PBS containing samples had a Dv50 smaller than 3 μm and over 50% particles fell into 1-5 μm size range. However, the span of these powders was greater than 8 due to the high Dv90. In contrast, the spans in SM samples were the smallest among all samples. The particle size was large in trehalose:leucine 90:10 SM buffer formulation. It might be a result of water absorption in the powder with 90% trehalose.

TABLE 1
Geometric particle size distribution of thin film freeze dried phage powders

| Formulation | Buffer | Dv10 (μm) | Dv50 (μm) | Dv90 (μm) | 1-5 μm % | Span |
|---|---|---|---|---|---|---|
| Trehalose: | PBS | 0.7 ± 0.0 | 1.7 ± 0.1 | 18.4 ± 4.4 | 50.9 ± 1.0 | 10.6 |
| Leucine | SM | 1.5 ± 0.0 | 7.0 ± 0.1 | 22.1 ± 0.5 | 30.8 ± 0.5 | 2.9 |
| 90:10 | No Buffer | 1.6 ± 0.4 | 8.6 ± 3.0 | 40 ± 26.6 | 27.7 ± 6.8 | 4.2 |
| Sucrose: | PBS | 0.7 ± 0.0 | 2.3 ± 0.2 | 19.5 ± 6.4 | 50.2 ± 1.7 | 8.0 |
| Leucine | SM | 1.3 ± 0 | 5.3 ± 0.2 | 15.5 ± 0.9 | 40.3 ± 1.7 | 2.7 |
| 75:25 | No Buffer | 1.1 ± 0.1 | 5.7 ± 0.5 | 25.5 ± 4.2 | 37.3 ± 2.5 | 4.3 |

Crystallinity. The physical states of TFFD phage powders were evaluation using XRD (FIG. 12). Characteristic peaks for NaCl were observed at 20 of 27.8°, 32° and 45.5° across all buffer containing formulations. In addition to NaCl characteristic peaks, SM buffer samples also have some relatively shorter peaks at 20 of 10.9°, 15.7°, and 21.8° to 23.7°, 26°, 27.5°, 39.3° to 43°. These could be contributed by the other components in the buffer, Tris and MgSO₄. The characteristic peaks for leucine, peaks at 20 of 7°, 19.2°, and 24.6°, were more pronounced in sucrose: leucine 75:25 samples as a result of the 15% more leucine comparing to trehalose:leucine 90:10 samples. No characteristic peaks for sucrose and trehalose were observed, indicating they turned to amorphous after the TFFD process. The two no-buffer powders exhibited to be amorphous as indicated by the broad ‘halo’ peak.

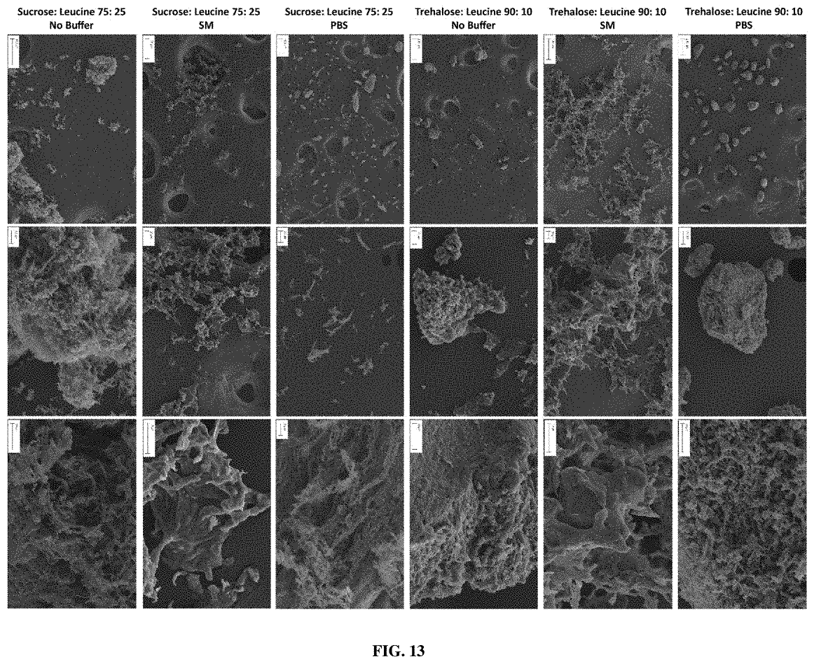

Morphology of powder. The surface morphology of the TFFD phage powders were analyzed using SEM (FIG. 13). Powders were generally highly porous, and network of nanostructured aggregates were observed in all samples. The size distributions in PBS containing samples were less homogeneous, as indicated by the high span in GPSD measurements. SM sample powders appeared most differently from the other groups. The particles exhibited branch-like structure and looks thinner. In a lower magnitude view, the particles seemed to have long extrusions and connected to each other. The surface of the SM powders was smoother, with small ‘bumps’ on the surface instead of network-like structures. The porosity of the powders can potentially improve the flowability of the powders and they tend to be broken down easily to nanoaggregates during the dispersion and impaction to lung.

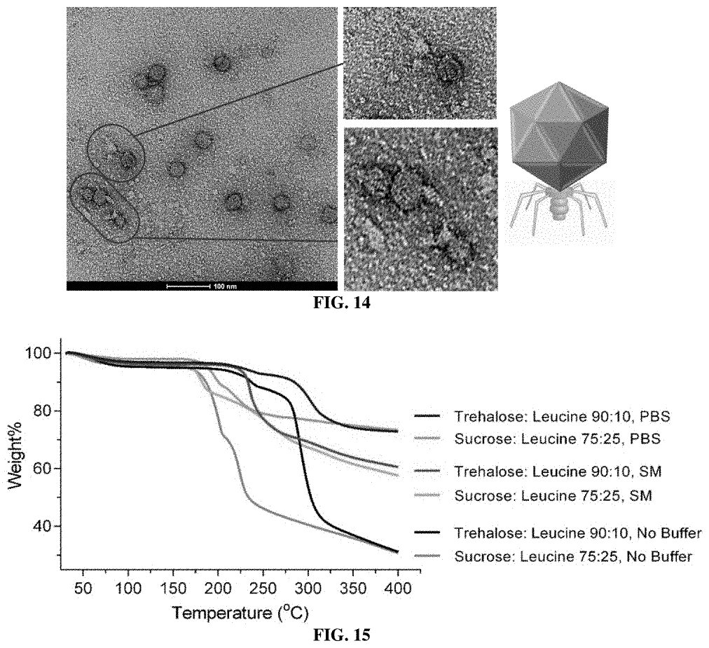

Morphology of phage. The morphology of T7 phage has already been well characterized. Basically, the phage is composed by an icosahedral (twenty faces) protein capsid with a relatively short tail, on which long tail fibers attached (as shown in the carton in FIG. 14.

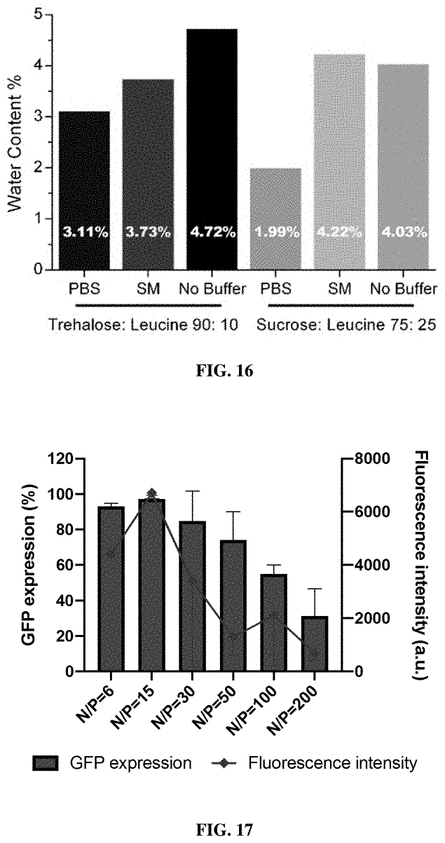

Water content and thermal analysis. TGA was used to map out the thermal stability profile (FIG. 15) of the TFFD phage powders and to determine the water content in the powders (FIG. 16). Water content was determined by identifying the weight loss at 120° C. The reliability of currently used method (ramping from 35° C. to 400° C. at a rate of 10° C./min) was confirmed by testing same sample with an isothermal heating method (ramping from 35° C. to 120° C. at a rate of 10° C./min followed by holding at 120° C. for 10 min). The results from both methods showed no significant difference (data not shown). Water contents were generally lower in buffer containing samples probably as a result of the existence of salt crystalline. PBS sample contain the least water and the moisture was high in one SM sample where sucrose: leucine was 75:25.

Conclusion. The feasibility of using TFFD technology to produce bioactive, inhalable phage powders has been demonstrated. It was proved that, with the optimal formulations, TFFD can successfully achieve micronized particles with minimum titer loss. It was demonstrated that incorporation of buffer system helps preserving phage stability as well as reducing the particle size to a more desired range.

TFFD is a desirable alternative to currently developed particle engineering methods given it eliminates stresses to phages from the vibration of nozzles in SD, SFD, and ASFD, and avoided the thermal stress in SD process. Therefore, development of bacteriophage inhalable dry powder using thin film freezing technology is a worthy strategy.

### Example 2—Development of Lipid Nanoparticles through Design of Experiments for Aerosolized Pulmonary Delivery of mRNA

### A. Material and Methods

1. Materials

DLin-MC3-DMA was purchased from Biofine International Inc., Vancouver, BC. 1,2-dipalmitoyl-sn-glycero-3-phosphocholine (DPPC), 1,2-dimyristoyl-rac-glycero-3-methoxypolyethylene glycol-2000 (DMG-PEG-2000), 1,2-distearoyl-sn-glycero-3-phosphocholine (DSPC), 1,2-distearoyl-sn-glycero-3-phosphoethanolamine-N-[Amino (Polyethylene Glycol) 2000 (DSPE-PEG 2000), and (Delta 9 cis)/1,2-dioleoyl-sn-glycero-3-phosphoethanolamine (DOPE) were purchased from Avanti Polar Lipids, AL, USA. N-(methylpolyoxyethyleneoxycarbonyl)-1,2-dimyristoyl-sn-glycero-3-phosphoethanolamine (DMPE-PEG 2000) was purchased from NOF Corporation, Tokyo, JP. Cholesterol was purchased from Sigma Aldrich, MO. Ethanol (molecular grade) was purchased from Decon Laboratories, Inc., PA. CleanCap® Enhanced Green Fluorescent Protein (EGFP) mRNA and CleanCap® Firefly luciferase (FLuc) mRNA were purchased from TriLink, San Diego, Calif., USA. Slide-A-Lyzer™ Gamma Irradiated Dialysis Cassette (10 kDa), Quanit-iT™ RiboGreen® RNA Reagent and Kit (Invitrogen), and Opti-MEM™ Reduced Serum Media (Gibco) were purchased from ThermoFisher Scientific Inc., Waltham, Mass., USA. Dulbecco's Modification of Eagle's Medium (DMEM), Fetal Bovine Serum (FBS), and Penicillin/Streptomycin (100×) were purchased from Corning, Manassas, Va., USA. Balb/c mice were purchased from Charles River Laboratories, Inc, Wilmington, Mass., USA.

2. Methods

Preparation of LNP formulations. Lipid nanoparticles containing EGFP mRNA or FLuc mRNA were prepared by combining an aqueous phase (mRNA diluted in 100 mM sodium acetate citrate buffer, pH 3.0) and an organic phase containing ethanol and lipids according to each formulation (Table 2) using a microfluidic mixer (Precision Nanosystems, Canada; Leung et al., 2015). After preparation, LNP formulations were dialyzed into 1× PBS (pH 7.4) for 2 hours in 10K MWCO Slide-A-Lyzer dialysis cassettes (Thermo Fisher Scientific, MA).

Measurements of size and zeta potential. The size and zeta potential of LNP formulations were characterized by using Zetasizer Nano-ZS (Malvern Instruments MA). Each formulation was 10-fold diluted in 0.1× PBS buffer for size measurement and 40-fold diluted in 0.1× PBS for zeta potential measurement. Dynamic light scattering was performed on diluted samples at 25° C. with 173° and the reported z-average diameter is a mean of three measurements.

mRNA Encapsulation efficiency. mRNA encapsulation efficiency was evaluated by low range Quanti-iT RiboGreen RNA reagent assay (Thermo Fisher Scientific, MA). Each LNP sample was diluted into TE buffer down to a mRNA concentration of 0.2 ng/μL. Aliquots of each LNP working solution was further diluted 1:1 in TE buffer (measuring unencapsulated mRNA) or 1:1 in TE buffer with 4% Triton-X100 (measuring total mRNA-both encapsulated within LNPs and unencapsulated free mRNA) in a 96-well plate. Samples were prepared in duplicate and 100 μL of 2000-fold diluted Quanti-iT™ RiboGreen RNA reagent was added to each sample the fluorescence intensity was measured by plate reader at excitation and emission wavelengths of 480 and 520 nm (Infinite M200, Tecan, Switzerland), respectively.

TNS assays. A series of buffers with pH ranging from 2.5 to 11 (pH 2.5, pH 3, pH 3.5, pH 4, pH 4.6, pH 5, pH 5.5, pH 5.8, pH 6, pH 6.5, pH 7, pH 7.5, pH 8, pH 8.5, pH 9, pH 9.5, pH 10, pH 10.5, pH 11) were prepared by adjusting the pH of a buffer solution consisting of 10 mM HEPES, 10 mM MES, 10 mM ammonium acetate, 130 mM NaCl with 1 N HCl. Also, 90 μL of each buffer solution was added to a 96-well plate. 2 μL of TNS stock solution (300 μM in DMSO) was added to the buffer solutions at different pH in the 96-well plate. Then 3 μL of an LNP solution (prepared with mRNA) was added to the above mixture. The fluorescence intensity was measured at an excitation wavelength of 325 nm and an emission wavelength of 435 nm. The fluorescence intensity was plotted against pH values and fitted using a three-parameter logistic equation (GraphPad Prism v.6, GraphPad Software). The pH value at which half of the maximum fluorescence is reached was regarded as the pKa of LNP formulations.

Aerosolization of LNP formulations. It has been shown that vibrating mesh nebulizers can be used to aerosolize shear susceptible formulations such as liposomes and niosomes and therefore are a good alternative to air jet and ultrasound nebulizers (Wagner et al., 2006; Elhissi et al., 2013). LNP formulations were added to the Aerogen Solo (Aerogen Ltd, Galway Ireland), which is a vibrating mesh nebulizer and the aerosol was subsequently collected by condensation in precooled tubes.

Cell culture. HEK-293 cells were cultured with Dulbecco's Modified Eagle Medium containing 10% FBS and 1% penicillin streptomycin. NuLi-1 cells (ATCC CRL-4013) were cultured in flasks pre-coated with 60 μg/mL solution of human placental collagen type IV (Sigma Aldrich, MO) and grown in bronchial epithelial growth medium (BEGM) supplemented with SingleQuot additives from Lonza (BEGM Bullet Kit, reference CC-3170) and 50 μg/mL G-418. All cell lines were maintained as monolayer cultures at 37° C. and 5% CO₂.

Intracellular uptake In vitro. Cells were seeded in 96-well plates at a cell density of 12,500 cells/well and grown for 24 hours at 37° C. and 5% CO₂. Then 10 μL of LNP at a 10 ng EGFP mRNA/μL concentration was added to cells in 0.2 mL cell culture media for 24 hours. After, the cell culture media was removed, and cells were washed with 1×PBS. To detach the cells, 100 μL of 0.25% trypsin-EDTA solution was added to each well and incubated at 37° C. for 8-10 minutes. Next, 100 μL of 1% FBS in Dulbecco's phosphate buffered saline was added, cells were spun at 125×g for 5 to 10 minutes and the supernatant was discarded. Cells were resuspended in 50 μL 1× PBS with 0.25 μL propidium iodide (PI) (1 mg/mL) solution. Cell percent GFP expression (i.e. transfection efficiency) and fluorescence intensity were analyzed by flow cytometry.

In vivo transfection. All animal protocols were approved by the Institutional Animal Care and Use Committee at the University of Texas at Austin. Balb/c mice (female, 6-8 weeks) were anesthetized under a continuous flow of 2% isoflurane, and approximately 50 μL of LNP containing 1.5 μg of FLuc mRNA/μL in PBS were administered intratracheally. After 6 hours, mice were intraperitoneally (i.p.) injected with D-Luciferin solution (30 mg/ml) to reach 150 mg Luciferin/kg body weight. After 15 minutes, mice were sacrificed and the lungs were carefully harvested and imaged by an In Vivo Imaging System (IVIS), with bioluminescence setting and a luminescent exposure time of 60 sec. Quantification of luminescence (in radiance [p/sec/cm²/sr]) was performed with Living Image 4.3 software (PerkinElmer).

Statistical analysis. The statistical analysis was performed using JMP 13. Data values are expressed as mean±standard deviations (SD). When required, one-way analysis of variance (one-way ANOVA) or Student's t-test was performed. *p-values≤0.05, **p-values≤0.01, ***p-values≤0.001, and ****p-values≤0.0001 were considered statistically significant.

### B. Results and Discussion

1. Results

Effects of N/P ratio on the efficacy of LNP formulations. To investigate the effects of the N/P ratio on intracellular uptake, six LNP formulations encapsulating EGFP mRNA were prepared by varying the N/P ratio between 6 to 200. LNP formulations were composed of DLin-MC3-DMA, a phosphatidylcholine (1,2-distearoyl-sn-glycero-3-phosphocholine, DSPC), cholesterol, and a PEG-lipid (polyethylene glycol-dimyristolglycerol, PEG-DMG) at a single molar ratio of 50:10:38.5:1.5, respectively (as previously described in Jayaraman et al., 2012). The different N/P ratios (N/P=6, 15, 30, 50, 100, and 200) were achieved by varying the relative amount of lipid composition added to the mRNA (10 ng/μl). LNPs were prepared and the intracellular uptake of each formulation was evaluated in HEK-293 cells by flow cytometry. As shown in FIG. 17, the LNP formulation with an N/P ratio=15 demonstrated both the highest percent GFP expression and mean fluorescence intensity. The intracellular uptake decreased as the N/P ratio increased from 15 to 200. The N/P ratio=15 was maintained for the following experiments, and this particular formulation is subsequently used in the experiments as the “reference formulation”.

DOE: Mixture experimental design with constraints. LNPs consist generally of four lipid components: ionizable lipid, phospholipid, PEG-lipid, and cholesterol. The different types and amount of lipids may affect the transfection efficacy of LNP formulations (Kauffman et al., 2015). One-factor-at-a-time design methods have been employed in several studies to investigate the effect of formulation composition on the efficacy of each LNP formulation (Belliveau et al., 2012; Akinc et al., 2009). However, this approach does not account for potential second-order interactions between composition parameters, which makes it less desirable for optimization of LNP formulations. Alternatively, fractional factorial design has been used to maximize the potency of LNP formulations for mRNA delivery (Kauffman et al., 2015). Although this method investigates second-order effects, the fact that not all variables can be included in the design is a major limitation. In order to systematically investigate the effects of variables on the potency of LNP formulations, a mixture design with constraints was employed in this study (Table 2). Using JMP software, a design of 18 LNP formulations was generated for testing (Table 3).

TABLE 2
Limits of experimental design space.

| | Lower limit | Upper limit |
|---|---|---|
| Component | (molar ratio) | (molar ratio) |
| Ionizable lipid | 0.4 | 0.6 |
| Phospholipid | 0.1 | 0.2 |
| PEG-lipid | 0.01 | 0.05 |
| Cholesterol | 0.15 | 0.49 |

TABLE 3
Composition of LNP formulations.

| | | | Molar composition | | | |
|---|---|---|---|---|---|---|
| | | | Dlin-MC3- | | PEG- | |
| Formulation # | Phospholipid | PEG-lipid | DMA | Phospholipid | lipid | Cholesterol |
| 1 | DOPE | DMG-PEG | 0.6 | 0.2 | 0.05 | 0.15 |
| 2 | DOPE | DMPE-PEG | 0.4 | 0.2 | 0.01 | 0.39 |
| 3 | DSPC | DMG-PEG | 0.5 | 0.14 | 0.01 | 0.35 |
| 4 | DOPE | DMPE-PEG | 0.6 | 0.15 | 0.03 | 0.22 |
| 5 | DSPC | DSPE-PEG | 0.4 | 0.2 | 0.05 | 0.35 |
| 6 | DPPC | DMG-PEG | 0.4 | 0.1 | 0.01 | 0.49 |
| 7 | DPPC | DSPE-PEG | 0.4 | 0.1 | 0.05 | 0.45 |
| 8 | DPPC | DMG-PEG | 0.6 | 0.2 | 0.01 | 0.19 |
| 9 | DOPE | DSPE-PEG | 0.6 | 0.1 | 0.05 | 0.25 |
| 10 | DOPE | DSPE-PEG | 0.4 | 0.2 | 0.03 | 0.37 |
| 11 | DPPC | DMPE-PEG | 0.6 | 0.2 | 0.01 | 0.19 |
| 12 | DSPC | DMG-PEG | 0.6 | 0.2 | 0.05 | 0.15 |
| 13 | DSPC | DSPE-PEG | 0.5 | 0.1 | 0.05 | 0.35 |
| 14 | DOPE | DSPE-PEG | 0.4 | 0.15 | 0.05 | 0.4 |
| 15 | DPPC | DSPE-PEG | 0.6 | 0.1 | 0.01 | 0.29 |
| 16 | DSPC | DMPE-PEG | 0.5 | 0.2 | 0.03 | 0.27 |
| 17 | DOPE | DMG-PEG | 0.4 | 0.16 | 0.01 | 0.43 |
| 18 | DPPC | DMPE-PEG | 0.4 | 0.1 | 0.03 | 0.47 |

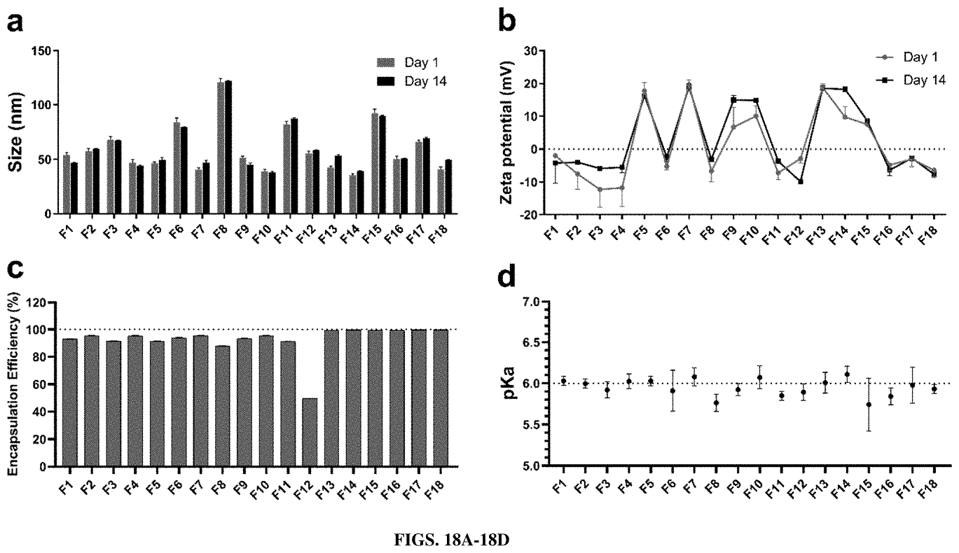

Characterization of mRNA-LNPs. Based on a mixture design with constraints, 18 formulations with an N/P ratio=15 were prepared using the NanoAssemblr® benchtop system. The size and zeta potential of the LNPs were evaluated by DLS. As shown in FIG. 18A, the particle size of the LNP formulations before nebulization varied from 35.7±1.1 nm (F14) to 120.9±3.4 nm (F8), while the zeta potential ranged from −12.2±5.5 mV (F3) to 18.8±1.2 mV (F13) (FIG. 18B). Furthermore, the size and zeta potential of the LNP formulations did not show significant changes after 14 days of storage in 4° C., which indicated that the size and surface charge of all formulations remained stable for at least 2 weeks (FIGS. 18A & 18B). The encapsulation efficiency of the formulations was evaluated by RiboGreen assay. Most of the formulations possessed a high encapsulation efficiency greater than 80%, except for F12 which showed 49% encapsulation efficiency (FIG. 18C). It has been previously reported that the pKa of LNPs may be critical for endosomal escape and has been implicated as a correlator for in vivo efficacy of gene therapy (Jayaraman et al., 2012). Therefore, the pKa of LNP formulations loaded with EGFP mRNA was measured using the TNS assay, and the pKa ranged from 5.74 (F15) to 6.11 (F14) (FIG. 18D).

To translate LNP formulations for clinical use, they must be able to be aerosolized for pulmonary delivery without significant instability. Towards that end, the effects of nebulization on the LNP formulations was investigated and the formulations that retained high intracellular uptake in vitro following nebulization were identified. LNP formulations were aerosolized by the Aerogen Solo nebulizer and the potency of each nebulized formulation was evaluated in human embryonic kidney HEK-293 and human bronchial epithelial NuLi-1 cell lines. After nebulization, the size of the LNP formulations ranged from 100.9 nm (F12) to 1480.7 nm (F7) and showed a significant increase compared to the pre-nebulized LNP formulations, while the zeta potential showed no significant changes amongst all formulations (FIGS. 19A-19C). It is worth noting that F8 had the smallest change in size upon nebulization, and F7 showed the largest change in size after nebulization. The encapsulation efficiency of the LNP formulations significantly decreased after nebulization, which indicated that the mRNA potentially leaked from the LNPs upon the nebulization process. The encapsulation efficiency of nebulized LNP formulations ranged from 15.5% (F12) to 79.9% (F17).

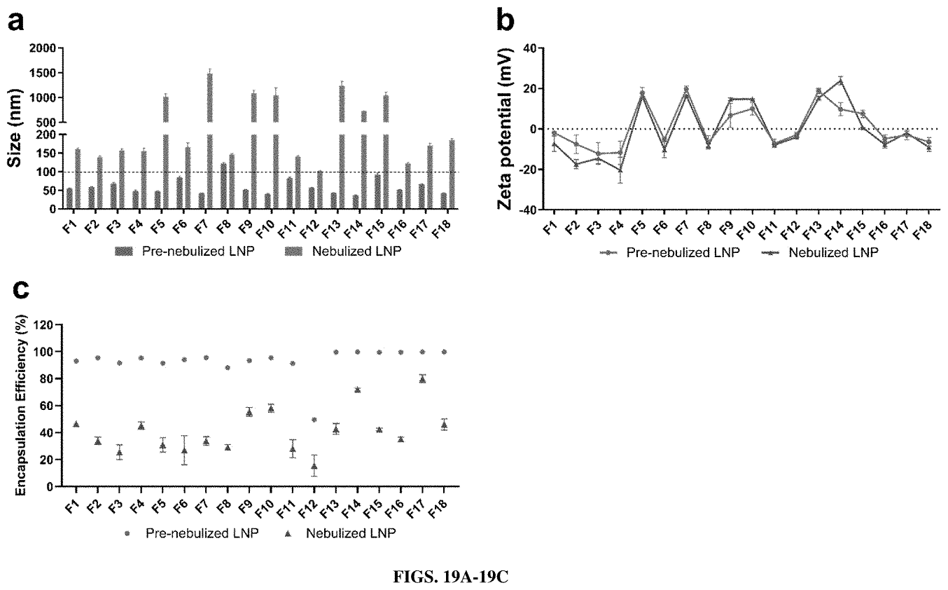

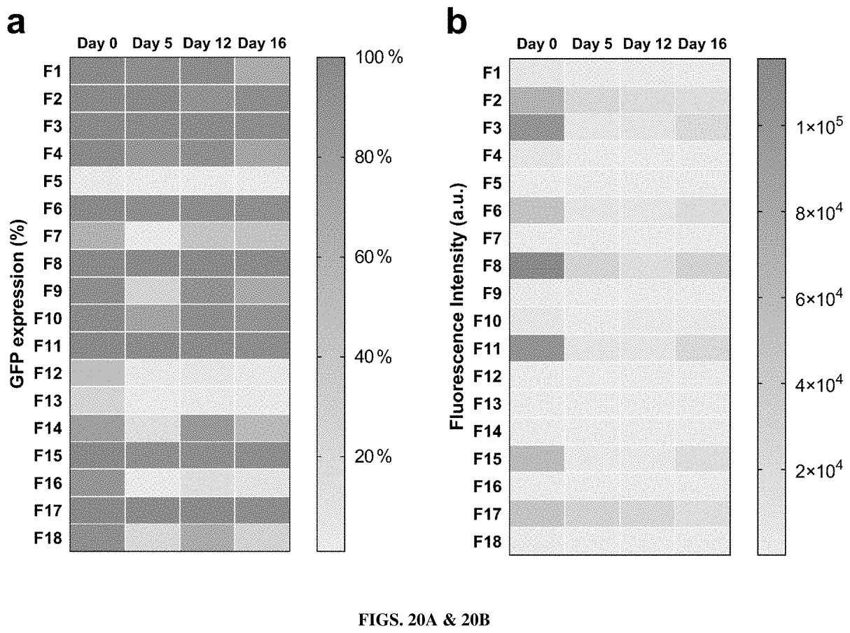

Intracellular uptake of LNP formulations in HEK-293 and NuLi-1 cells. The intracellular uptake of pre- and post-nebulized LNP mRNA formulations was assessed using flow cytometry by measuring percent GFP expression and fluorescence intensity in HEK-293 and NuLi-1 cell lines. On day 0 (i.e. incubation same day as preparation of formulations), the intracellular uptake of each mRNA-encapsulated formulation was measured in HEK-293 cells to identify formulations that exhibited higher transfection than the reference formulation (DLin-MC3-DMA:DSPC:cholesterol:PEG-DMG=50:10:38.5:1.5, N/P=15). It was found that most formulations showed over 50% GFP expression, except F5, F12, and F13. Notably, although most formulations had relatively high percent GFP expression, the intracellular uptake in terms of fluorescence intensity varied among the formulations. Eight out of 18 formulations (F2, F3, F4, F6, F8, F11, F15 and F17) showed a significantly higher fluorescence intensity compared to the reference formulation, which showed a mean fluorescence intensity of 6708 a.u. in HEK-293 cells on Day 0. The percent GFP expression of these eight formulations were as high as over 95% and showed no significant differences when compared to the reference formulation. Next, the stability of LNPs (i.e. lack of premature mRNA leakage) was tested by quantifying their intracellular uptake after 0, 5, 12, and 16 days of refrigerated storage. As shown in FIG. 20A, eight formulations (F2, F3, F6, F8, F10, F11, F15 and F17) remained stable in terms of percent GFP expression after 16 days of storage at 4° C. In contrast, the fluorescence intensity of all formulations decreased significantly after 5 days of storage at 4° C. (FIG. 20B). Specifically, F2, F3, F6, F8, F11, F15, and F17 showed a fluorescence intensity of over 18,000 a.u. which were significantly higher than the reference formulation after 16 days of storage.

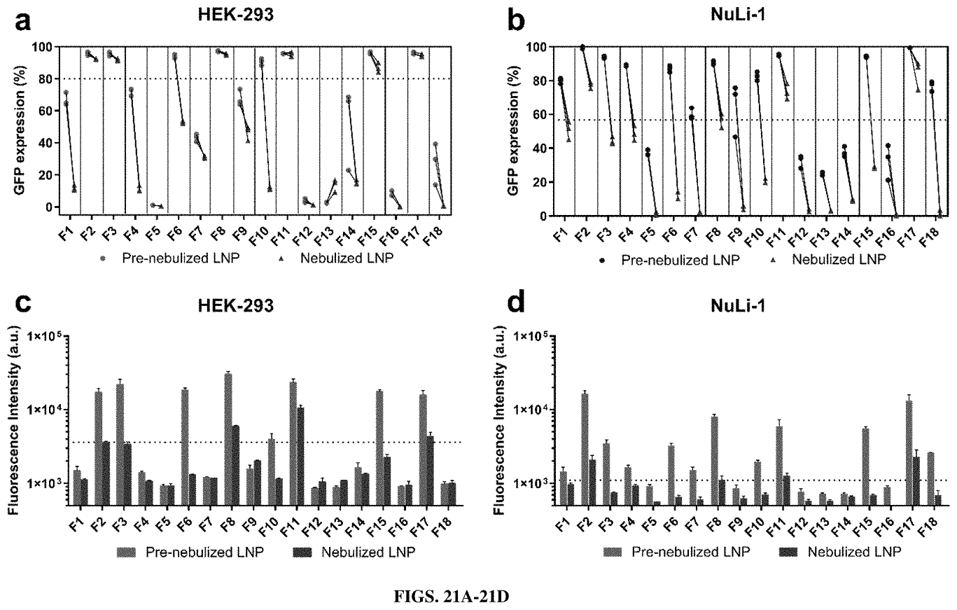

Upon nebulization, all LNP formulations showed significantly decreased fluorescence intensity compared to pre-nebulized LNP formulations in both HEK-293 cells and NuLi-1 cells. This finding indicates that the aerosolization process negatively affected the mRNA transfection in vitro. It was found that F2, F3, F8, F11, and F17 showed no significant changes in terms of percent GFP expression after nebulization compared to pre-nebulized LNPs (FIGS. 21A & 21C). Despite a significant decrease of fluorescence intensity observed in all LNP formulations, the aforementioned five formulations retained relatively high fluorescence intensity (over 3000 a.u.) upon nebulization (FIGS. 21B & 21D). In NuLi-1 cells, although F2, F8, F11, and F17 showed decreased percent GFP expression and fluorescence intensity upon nebulization, these four formulations still demonstrated relatively high GFP expression (over 50%) and fluorescence intensity (over 1000 a.u.) compared to other formulations. In summary, four formulations (F2, F8, F11 and F17) with relatively high intracellular uptake after 16 days of storage and nebulization were identified.

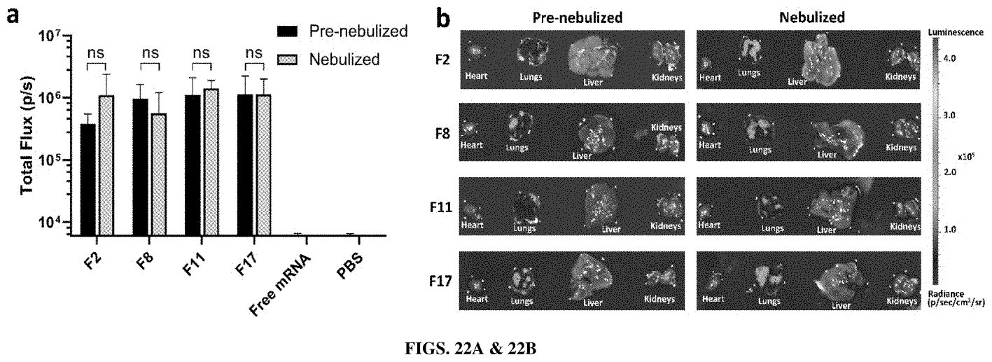

Intratracheal delivery of LNP formulations to mice. Based on intracellular uptake in vitro, four lead formulations (F2, F8, F11 and F18) were selected for further study in vivo. Specifically, firefly luciferase (Luc) mRNA was loaded into these LNP formulations and nebulized by an Aerogen Solo nebulizer. The collected nebulized dispersions were compared to pre-nebulized controls using intratracheal instillation administration to lungs of mice to investigate in vivo transfection and biodistribution. After 6 h post-administration, luciferase activity was predominantly detected in the lung compared to other organs for the four lead formulations, irrespective of the nebulization process (FIG. 22). Interestingly, there was no statistically significant difference in luminescence intensity between mice dosed with either pre-nebulized or nebulized LNP formulations, which indicated that the candidate formulations retained their function after nebulization.

2. Discussion

This work highlights a DOE approach to discover LNP formulations that are suitable for aerosolized delivery of mRNA. Using DOE, 18 formulations of various lipid compositions were prepared and characterized in terms of physicochemical properties and intracellular uptake. Four lead formulations that had relatively higher intracellular uptake before and after nebulization were identified and intratracheally delivered to mice, where they showed the ability to deliver mRNA to lungs in vivo before and after nebulization. Extensive statistical analysis of formulations helped identify certain parameters that impacted stability and intracellular delivery of nanoparticles.

Composition of LNP formulations influenced their physicochemical properties (size, zeta potential, and encapsulation efficiency) before and after nebulization. It was found that pre-nebulized dispersions had a particle size that was dependent on the molar ratio of PEG-lipid used. In these pre-nebulized formulations, it appeared that the type of PEG-lipid used did not influence particle size in a significant way. In contrast, the nebulized dispersions were significantly influenced by the type of PEG-lipid used in the formulation. These observations are discussed below.

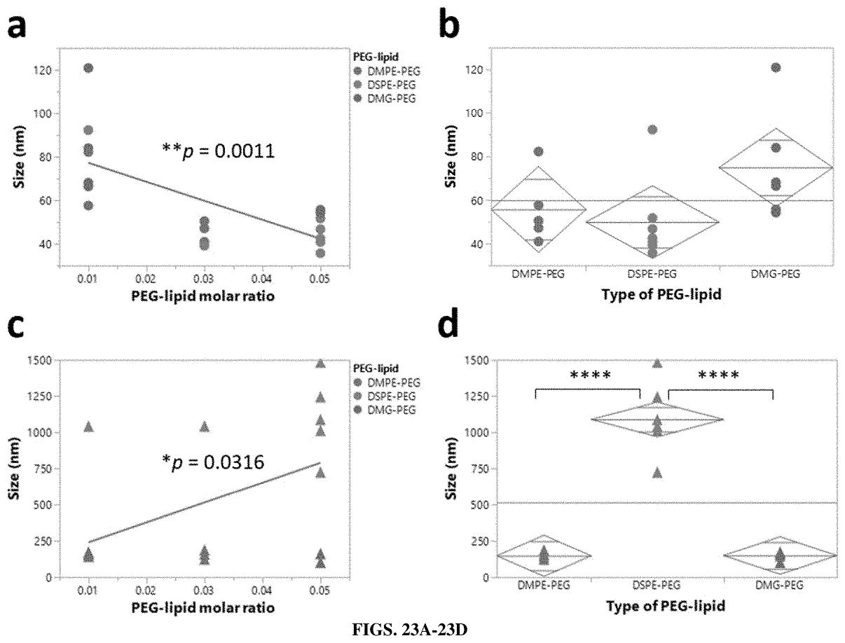

To explore the correlation between LNP size and each LNP component, the size of the LNPs before and after nebulization was plotted against each component, and the orthogonal trend was analyzed. A statistically significant (p<0.05) trend of decreasing size was observed with increasing molar PEG-lipid composition for pre-nebulized LNP formulations, independent of the other formulation parameters (FIG. 23A). The size was not significantly correlated to other components of the formulation in terms of molar amounts. Similar findings have been reported where PEGylated liposomes showed a significant decrease in size compared to conventional liposomes, and that the increasing the overall amount of DSPE-PEG led to a decrease in liposome size (Kontogiannopoulos et al., 2014; Sriwongsitanont and Ueno, 2004). A potential explanation for this finding could be due to the fact that lateral repulsion of the surface of lipid bilayers increases by extensive hydration around the head group with an increasing concentration of PEG-lipid (Akinc et al., 2009). To reduce the high lateral repulsion, particle sizes must decrease, which subsequently increases the curvature of the grafting surface (Sriwongsitanont et al., 2004). In contrast, as shown here in post-nebulization formulations, a statistically significant increase in particle size was observed with increasing molar amounts of PEG-lipid (FIG. 23C). This is likely due to the type of PEG-lipid used in the formulation rather than the PEG-lipid molar ratio. From the data in FIG. 23C rearranged by type of PEG-lipid (FIG. 23D), formulations with DSPE-PEG showed a larger size compared to formulations with the other two types of PEG-lipid, which indicated that the type of PEG-lipid significantly affected the size of LNP after nebulization (FIG. 23D). These results indicated that formulations made with DSPE-PEG had a poor ability to maintain their size after the aerosolization process.

The zeta potential of the formulations, both before and after nebulization, was also primarily driven by the type of PEG-lipid selected. A statistically significant trend of increasing LNP zeta potential was observed with an increasing molar ratio of PEG-lipid for either pre-nebulized or nebulized LNP formulations, independent of the other formulation parameters (FIGS. 24A & 24C). However, it is worth noting that this significant trend was primarily related to the type of PEG-lipid used, where formulations with DSPE-PEG showed a higher zeta potential irrespective of aerosolization process (FIGS. 24B & 24D).

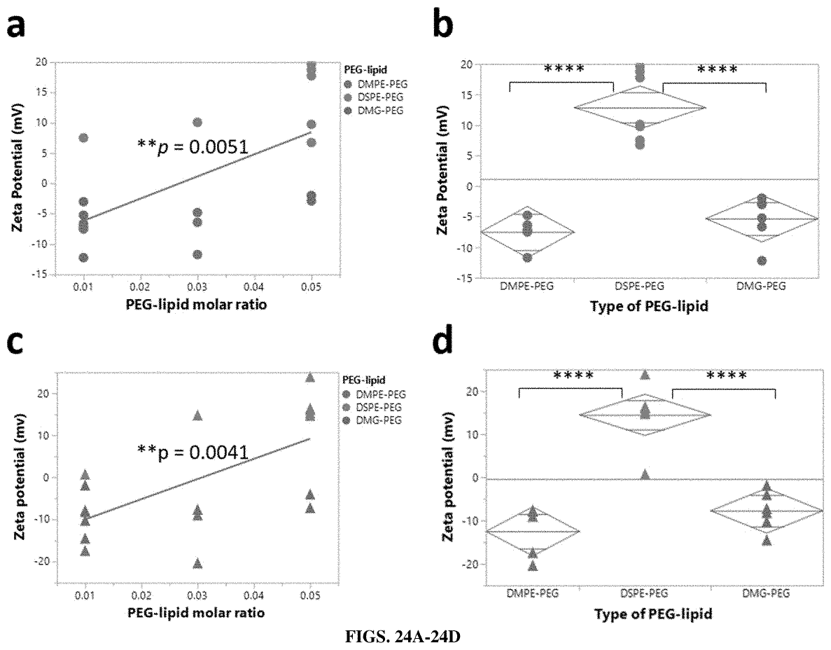

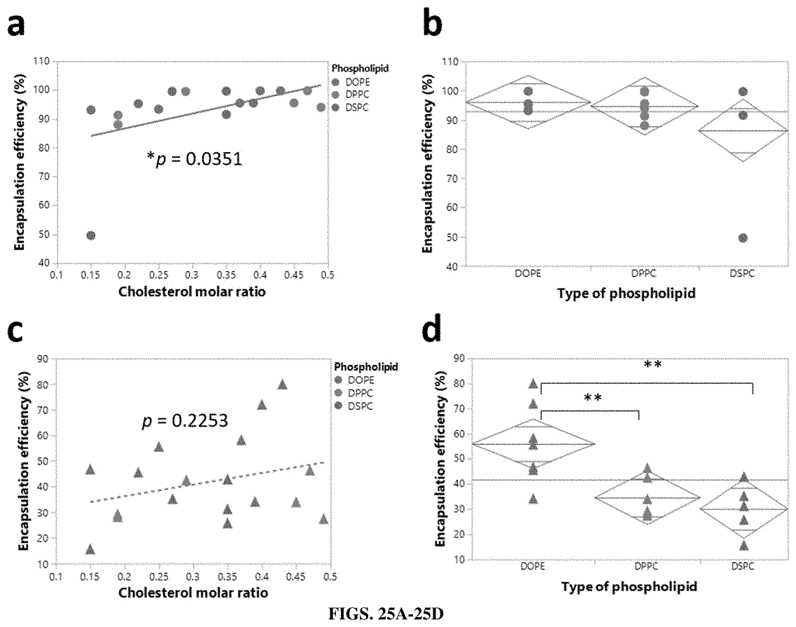

With respect to encapsulation efficiencies, almost all the formulations achieved high encapsulation efficiency. It was found that an increasing cholesterol molar ratio resulted in a statistically significant increase in the encapsulation efficiency for the pre-nebulized LNPs (FIG. 25A). This indicated that the structural cholesterol played an important role in the encapsulation efficiency of LNP formulations before aerosolization, while the type of phospholipid used did not demonstrate significant effects before nebulization (FIG. 25B). Li et al. have reported that lipid-like nanoparticles with higher molar ratios of cholesterol possessed a higher encapsulation efficiency of mRNA (Li et al., 2015). However, after nebulization, the type of phospholipid, instead of the molar amount of cholesterol, became the only factor that significantly influenced the encapsulation efficiency (FIGS. 25C & 25D). LNP formulations with DOPE showed a significantly higher encapsulation efficiency compared to LNP formulations with either DSPC or DPPC (FIG. 25D). This finding indicated that the inclusion of DOPE could significantly enhance the ability of LNPs to prevent mRNA from leaking during the aerosolization process.

PEG-lipid molar ratio negatively influenced the intracellular uptake of LNPs before and after nebulization. Formulations of the mRNA loaded LNPs must balance several performance measures, such as transfection efficiencies and nanoparticle stability. In the formulations developed in this study, PEG-lipids were used to impart physical stability on the nanoparticle dispersion. However, it has been shown that PEGylation can significantly influence transfection efficiencies (Otsuka et al., 2003; Mishra et al., 2004; Osman et al., 2018). Here, the PEG-lipid molar ratio significantly and negatively affected the intracellular uptake of LNPs both before and after nebulization.

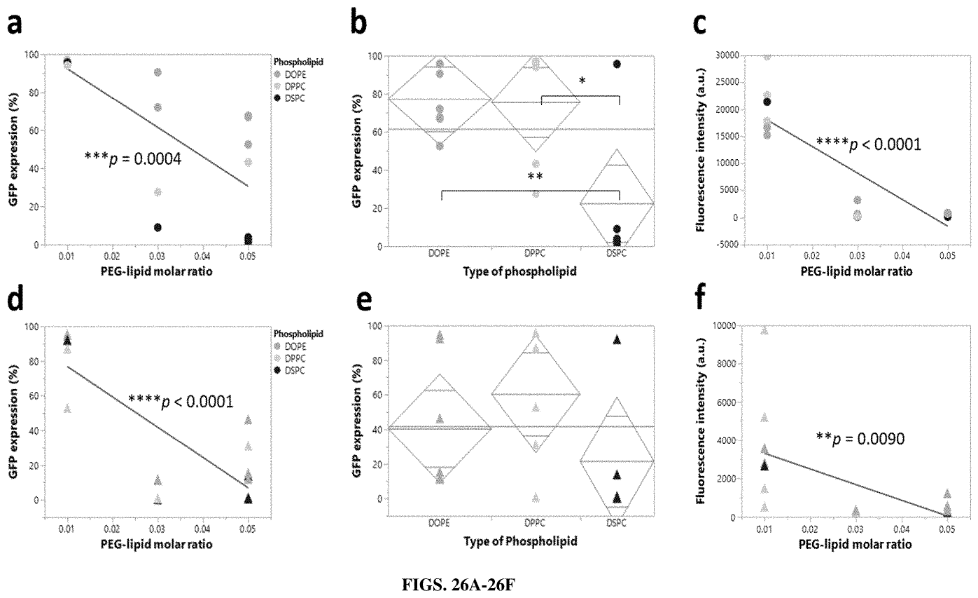

Specifically, increasing the PEG-lipid molar ratio negatively affected the intracellular uptake of pre-nebulized LNPs in HEK-293 cells (FIGS. 26A & 26C) and NuLi-1 cells (data not shown). A statistically significant trend of decreasing percent GFP expression and fluorescence intensity was observed with an increasing molar fraction of PEG-lipid, independent of the other formulation parameters; this finding was consistent with previous reports (Otsuka et al., 2003; Mishra et al., 2004). In addition, the type of phospholipid significantly influenced percent GFP expression. It was observed that LNP formulations with DSPC showed significantly lower percent GFP expression compared to LNP formulations with either DOPE or DPPC (FIG. 26B), an observation consistent with previous reports (Kauffman et al., 2015). Upon nebulization, increasing the molar ratio of PEG-lipid resulted in the same observed trend in HEK-293 (FIGS. 26D & 26F) and NuLi-1 cells (data not shown), but there were no significant effects of the type of phospholipid on percent GFP expression (FIG. 26E).

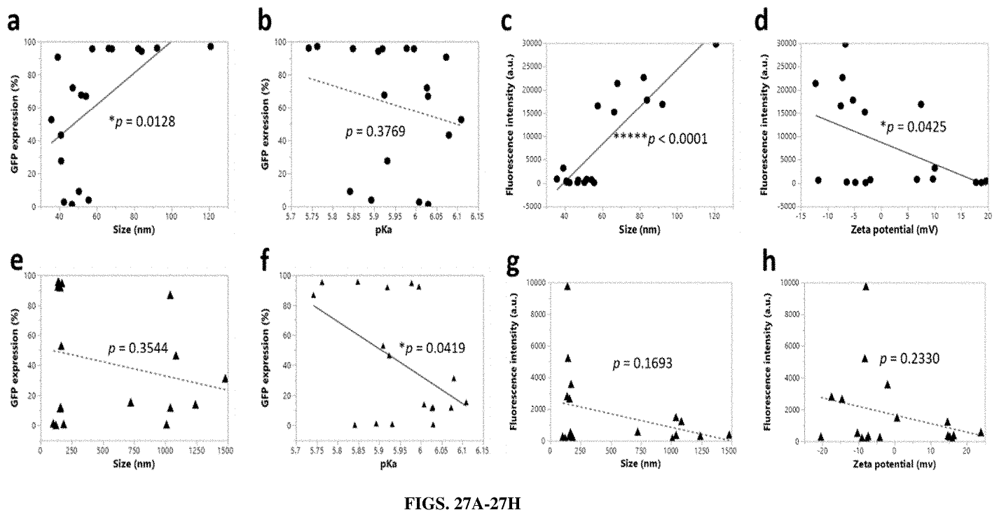

Correlation between physicochemical properties and intracellular uptake before and after nebulization. In order to explore the correlation between physicochemical properties and the potency of LNP formulations, size, zeta potential, encapsulation efficiency, and pKa were plotted against intracellular uptake and fluorescence intensity in HEK-293 cells. It was found that LNP formulations with a larger particle size showed a higher percent GFP expression and fluorescence intensity before nebulization (FIGS. 27A & 27C) as a significant trend of an increased percent GFP expression and fluorescence intensity was observed with an increased particle size (FIG. 27A). Furthermore, pre-nebulized formulations with a higher zeta potential showed a lower fluorescence intensity (FIG. 27D). After nebulization, the pKa appeared to be the significant parameter influencing percent GFP expression, whereby a lower pKa led to a higher percent GFP expression (FIG. 27F), while other parameters showed no significant effects on the intracellular uptake.

Conclusion. The in vitro performance formulations of LNPs for aerosol gene delivery are significantly influenced by lipid composition. Using the DOE approach for formulation discovery, four lead formulations that had relatively higher intracellular uptake before and after nebulization were identified and subsequently tested in vivo. These formulations when intratracheally delivered to mice, showed the ability to deliver mRNA to lungs in vivo both before and after nebulization. Extensive statistical analysis of formulations helped identify certain parameters that impacted stability and intracellular delivery of nanoparticles. DSPE-PEG was a negative factor for the stability of LNP nanoparticles as a significantly higher aggregate level appeared after nebulization compared to formulations with DMG-PEG and DMPE-PEG. It was also found that the PEG-lipid molar ratio and DSPC phospholipid significantly and negatively affected the intracellular uptake of LNPs. From this approach, LNP formulations can be more rapidly and easily identified that possess the optimal properties to facilitate effective aerosolized delivery of mRNA. While this work focused on the delivery of mRNA towards the treatment of pulmonary diseases, the DOE strategy could be broadly applied to discover LNP compositions and their properties that promote enhanced delivery of nucleic acid therapeutics for different indications.

### C. Further Comparative Examples

1. Materials

1,2-dioleoyl-3-trimethylammonium-propane (DOTAP), 1,2-dioleoyl-3-dimethylammonium-propane (DODAP), 1,2-dipalmitoyl-sn-glycero-3-phosphocholine (DPPC), 1,2-distearoyl-sn-glycero-3-phosphocholine (DSPC), 1,2-distearoyl-sn-glycero-3-phosphoethanolamine-N4Amino (Polyethylene Glycol) 2000 (DSPE-PEG 2000), and (Delta 9 cis)/1,2-dioleoyl-sn-glycero-3-phosphoethanolamine (DOPE) were purchased from Avanti Polar Lipids, AL, USA. N-(methylpolyoxyethyleneoxycarbonyl)-1,2-dimyristoyl-sn-glycero-3-phosphoethanolamine (DMPE-PEG 2000) was purchased from NOF Corporation, Tokyo, JP. Cholesterol was purchased from Sigma Aldrich, MO. Ethanol (molecular grade) was purchased from Decon Laboratories, Inc., PA. Edit-R Cas9 Nuclease mRNA with EGFP reporter (reference CAS11860) was purchased from Horizon Discovery Dharmacon Inc., Chicago, Ill., USA. Slide-A-Lyzer™ Gamma Irradiated Dialysis Cassette (10 kDa), Quanit-iT™ RiboGreen® RNA Reagent and Kit (Invitrogen), and Opti-MEM™ Reduced Serum Media (Gibco) were purchased from ThermoFisher Scientific Inc., Waltham, Mass., USA. Dulbecco's Modification of Eagle's Medium (DMEM), Fetal Bovine Serum (FBS), and Penicillin/Streptomycin (100×) were purchased from Corning, Manassas, Va., USA.

2. Methods

Preparation of LNP formulations. Lipid nanoparticles containing Edit-R Cas9 Nuclease mRNA were prepared by combining an aqueous phase (mRNA diluted in 50 mM sodium acetate citrate buffer, pH 4.0) and an organic phase containing ethanol and lipids according to each formulation (Table 1) using a microfluidic mixer (Precision Nanosystems, Canada; Leung et al., 2015). Flow ratio was 3:1 (aqueous:organic) and the nitrogen to phosphorus (N/P) ratio was 6. After preparation, LNP formulations were dialyzed into 1× PBS (pH 7.4) for 2 hours in 10K MWCO Slide-A-Lyzer dialysis cassettes (Thermo Fisher Scientific, MA).

Measurements of size and zeta potential. The size and zeta potential of LNP formulations were characterized by using Zetasizer Nano-ZS (Malvern Instruments MA). Each formulation was 10-fold diluted in 0.1× PBS buffer for size measurement and 40-fold diluted in 0.1× PBS for zeta potential measurement. Dynamic light scattering was performed on diluted samples at 25° C. with 173° and the reported z-average diameter is the mean of three measurements.

mRNA Encapsulation efficiency. mRNA encapsulation efficiency was evaluated by low range Quanti-iT RiboGreen RNA reagent assay (Thermo Fisher Scientific, MA). Each LNP sample was diluted into TE buffer down to a mRNA concentration of 0.2 ng/μL. Aliquots of each LNP working solution was further diluted 1:1 in TE buffer (measuring unencapsulated mRNA) or 1:1 in TE buffer with 4% Triton-X100 (measuring total mRNA-both encapsulated within LNPs and unencapsulated free mRNA) in a 96-well plate. Samples were prepared in duplicate and 100 μl of 2000-fold diluted Quanti-iT™ RiboGreen RNA reagent was added to each sample the fluorescence intensity was measured by plate reader at excitation and emission wavelengths of 480 and 520 nm (Infinite M200, Tecan, Switzerland), respectively.

Cell culture. HEK-293 cells were cultured with Dulbecco's Modified Eagle Medium containing 10% FBS and 1% penicillin streptomycin. NuLi-1 cells (ATCC CRL-4013) were cultured in flasks pre-coated with 60 μg/mL solution of human placental collagen type IV (Sigma Aldrich, MO) and grown in bronchial epithelial growth medium (BEGM) supplemented with SingleQuot additives from Lonza (BEGM Bullet Kit, reference CC-3170) and 50 μg/mL G-418. All cell lines were maintained as monolayer cultures at 37° C. and 5% CO₂.

Intracellular uptake in vitro. Cells were seeded in 96-well plates at a cell density of 12,500 cells/well and grown for 24 hours at 37° C. and 5% CO₂. Then 10 μL of LNP at a 10 ng EGFP mRNA/μL concentration was added to cells in 0.2 mL cell culture media for 24 hours. After, the cell culture media was removed, and cells were washed with 1×PBS. To detach the cells, 100 μL of 0.25% trypsin-EDTA solution was added to each well and incubated at 37° C. for 8-10 minutes. Next, 100 μL of 1% FBS in Dulbecco's phosphate buffered saline was added, cells were spun at 125×g for 5 to 10 minutes and the supernatant was discarded. Cells were resuspended in 50 μL 1× PBS with 0.25 μL propidium iodide (PI) (1 mg/mL) solution. Cell percent GFP expression (i.e. transfection efficiency) and fluorescence intensity were analyzed by flow cytometry.

3. Results

Based on a mixture design with constraints, 20 formulations with an N/P ratio=6 were prepared using the NanoAssemblr® benchtop system (Table 4).

TABLE 4
Composition of LNP formulations.

| | | | | Molar composition | | | |
|---|---|---|---|---|---|---|---|
| | Cationic | | | Cationic | | PEG- | |
| Formulation # | lipid | Phospholipid | PEG-lipid | lipid | Phospholipid | lipid | Cholesterol |
| 1 | DOTAP | DPPC | DSPE-PEG | 0.44 | 0.2 | 0.05 | 0.31 |
| 2 | DODAP | DPPC | DMPE-PEG | 0.5 | 0.2 | 0.01 | 0.29 |
| 3 | DODAP | DOPE | DSPE-PEG | 0.4 | 0.2 | 0.01 | 0.39 |
| 4 | DOTAP | DOPE | DMPE-PEG | 0.4 | 0.2 | 0.01 | 0.39 |
| 5 | DODAP | DPPC | DSPE-PEG | 0.6 | 0.2 | 0.05 | 0.15 |
| 6 | DODAP | DOPE | DSPE-PEG | 0.5 | 0.1 | 0.05 | 0.35 |
| 7 | DODAP | DOPE | DMPE-PEG | 0.6 | 0.2 | 0.01 | 0.19 |
| 8 | DODAP | DOPE | DMPE-PEG | 0.5 | 0.1 | 0.01 | 0.39 |
| 9 | DOTAP | DOPE | DSPE-PEG | 0.6 | 0.2 | 0.05 | 0.15 |
| 10 | DOTAP | DOPE | DSPE-PEG | 0.6 | 0.1 | 0.01 | 0.29 |
| 11 | DOTAP | DPPC | DMPE-PEG | 0.6 | 0.1 | 0.01 | 0.29 |
| 12 | DODAP | DOPE | DMPE-PEG | 0.4 | 0.2 | 0.05 | 0.35 |
| 13 | DOTAP | DPPC | DMPE-PEG | 0.6 | 0.2 | 0.05 | 0.15 |
| 14 | DODAP | DPPC | DMPE-PEG | 0.4 | 0.1 | 0.05 | 0.45 |
| 15 | DOTAP | DPPC | DSPE-PEG | 0.6 | 0.2 | 0.01 | 0.19 |
| 16 | DODAP | DPPC | DSPE-PEG | 0.4 | 0.1 | 0.01 | 0.49 |
| 17 | DOTAP | DPPC | DSPE-PEG | 0.4 | 0.1 | 0.01 | 0.49 |
| 18 | DOTAP | DOPE | DMPE-PEG | 0.4 | 0.1 | 0.05 | 0.45 |
| 19 | DODAP | DOPE | DMPE-PEG | 0.6 | 0.1 | 0.05 | 0.25 |
| 20 | DOTAP | DPPC | DSPE-PEG | 0.6 | 0.1 | 0.05 | 0.25 |

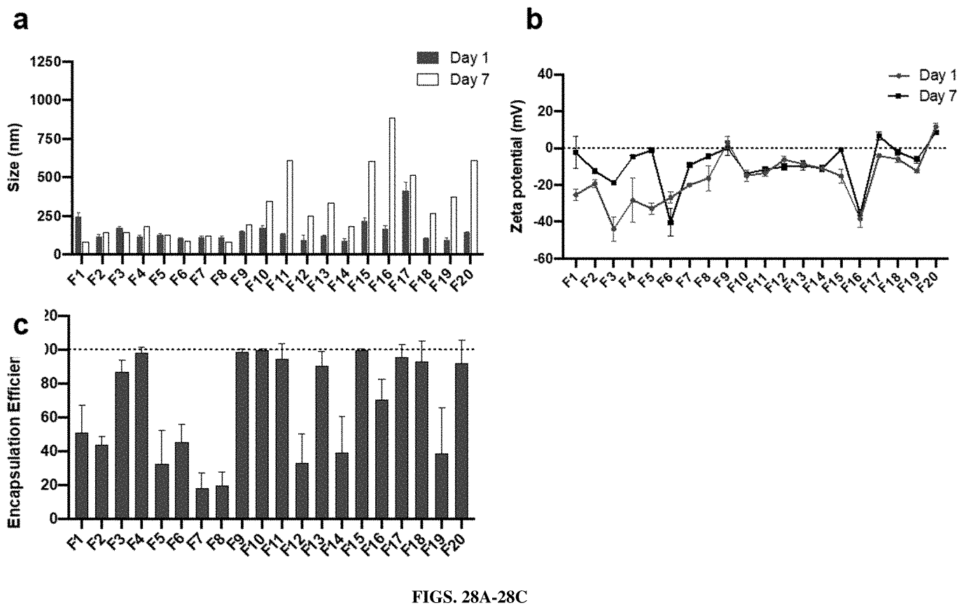

Characterization of mRNA-LNPs. The size and zeta potential of the LNPs were evaluated by dynamic light scattering (DLS) (Zetasizer Nano, Malvern Instruments, MA). Size and zeta potential measurements were performed in 0.1× PBS at 25° C. and a scattering angle of 173°. As shown in FIG. 28A, the particle size of the LNP formulations on day 1 varied from 83.3±14.7 nm (F8) to 416.30±41.1 nm (F17), while the zeta potential ranged from −43.95±4.75 mV (F3) to 11.7±1.4 mV (F20) (FIG. 28B). However, the size and zeta potential of the LNP formulations showed changes after 7 days of storage at 4° C., with an increase in particle size and changes in zeta potential for some formulations (FIGS. 28A & 28B). The encapsulation efficiency of the formulations was evaluated by RiboGreen assay according to the manufacturer protocol (Thermo Fisher Scientific, MA). Half of the formulations possessed a high encapsulation efficiency greater than 80% (F3, F4, F9, F10, F11, F13, F15, F17, F18, and F20), and F16 demonstrated an encapsulation efficiency of 70.28%. However, the other formulations demonstrated encapsulation efficiencies equal or lower than 50% (FIG. 28C).

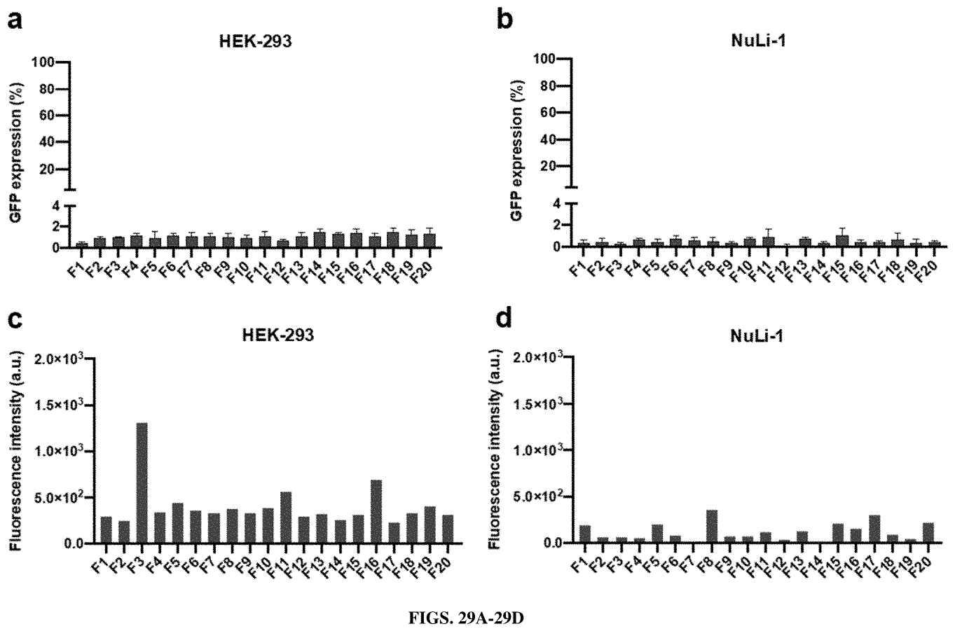

Intracellular uptake of LNP formulations in HEK-293 and NuLi-1 cells. The intracellular uptake of LNP mRNA formulations after 24 hs was assessed using flow cytometry by measuring percent GFP expression and fluorescence intensity in HEK-293 and NuLi-1 cell lines. It was found that all formulations showed less than 2% GFP expression (FIGS. 29A & 29B). Notably, although most formulations had relatively low percent GFP expression, the intracellular uptake in terms of fluorescence intensity varied among the formulations. F3 showed a significantly higher fluorescence intensity compared to F2, F14, and F17 in HEK-293 cells (FIG. 29C, p<0.05), but showed no significant differences in fluorescence intensities when tested in NuLi-1 cells (FIG. 29D).

### Example 3—Development of PEGylated Chitosan/CRISPR-Cas9 and Lipid Nanoparticle-mRNA Powders for Pulmonary Delivery via Thin Film Freezing

### A. Material and Methods

1. Materials

Poly (ethylene glycol) monomethyl ether MW 5000 kDa, mannitol, sucrose, trehalose, and leucine were purchased from Sigma-Aldrich (St. Louis, Mo., USA). Low molecular weight chitosan MW 15 kDa, was obtained from Polysciences Inc., USA. Nuclease-free water, Dulbecco's Modified Eagle's Medium (DMEM), Opti-MEM, and diethyl ether were obtained from Thermo Fisher Scientific Inc. (Waltham, Mass., USA). pSpCas9(BB)-2A-GFP (PX458) was a gift from Feng Zhang (Addgene plasmid #48138; http://n2t.net/addgene:48138; RRID:Addgene_48138; Ran et al., 2013).

2. Methods

Preparation of Dry Powder for Inhalation by TFF. Mannitol, sucrose, or trehalose at different concentration (10%-0.1%, w/v), and 0.3% leucine were mixed in PEGylated chitosan/DNA nanocomplexes which was prepared by the previously reported method (Zhang et al., 2018). Fluorescein sodium salt (0.02%) was added to formulations for in vitro aerodynamic performance evaluation. Approximately 15 μL of liquid was dropped from a height of 10 cm onto a rotating cryogenically cooled (−70° C.) stainless steel drum cooled by liquid nitrogen. The frozen samples were collected in a stainless-steel container filled with liquid nitrogen and transferred into a −80° C. freezer to remove extra liquid nitrogen. A VirTis Advantage Lyophilizer (VirTis Company Inc., Gardiner, N.Y.) was used to remove the water. The samples were kept at −40° C. for 40 h for primary drying, and the temperature was slowly increased to 25° C. over 650 min, and then kept at 25° C. for another 6 h to dry for secondary drying. The pressure was kept at 300 mTorr during the drying process. Four lipid nanoparticle dry power formulations were also formulated with mannitol, sucrose, and trehalose at a concentration of 20% (w/v).

Measurements of Size and Zeta Potential. Dry powder formulations were reconstituted in sterile nuclease-free water. The hydrodynamic diameter and zeta potential of reconstituted formulations were measured in triplicate by Zetasizer Nano ZS (Malvern Instruments, UK) at 25° C. Briefly, 20 μL of the nanocomplexes was added to 80 μL of sodium acetate buffer at pH 5.5 and mixed thoroughly before measurements.

Measurements of Geometric Particle Size Distribution. Geometric particle size distribution of refined dry powder formulations was evaluated by a HELOS laser diffraction instrument (Sympatec GmbH, Germany) using RODOS dispersion at 3 bar. Measurements were taken every 10 ms following powder dispersion. Measurements with optical density ranging from 5 to 25% were averaged to determine geometric particle size distribution.

Scanning electron microscopy (SEM). The surface morphology of 6 refined dry powder formulations were assessed by SEM (Zeiss Supra 40 VP SEM, Carl Zeiss Microscopy GmbH, Jena, Germany). Dry powder samples were mounted onto aluminum SEM stubs covered by carbon tape and sputter coated with 12 nm of platinum/palladium (Pt/Pd) by a Cressington sputter coater 208HR (Cressington Scientific Instruments Ltd., Watford, U.K.) before the image capture.

X-Ray Powder Diffraction. The crystallinity of CSP7 was identified by X-ray diffractometer (MiniFlex 600, Rigaku Co., Japan) under ambient conditions. Powders were placed on the glass slides and the scattered intensity was collected from 5 to 40° 20 (step size of 0.025°, 2°/min, Cu Kα radiation at 15 mA and 40 kV). Crystallinity was analyzed and calculated by Jade 9 software (KS Analytical Systems, Aubrey, Tex.).

Aerodynamic Particle Size Distribution by next generation impactor (NGI). In vitro aerodynamic performance was detected by the Next Generation Impactor (NGI, MSP Corporation, MN, USA). Dry powders were loaded into size 3 hypromellose (HPMC) capsules, a gift from Capsugel Inc. (Morristown, N.J., US). Dry powder formulations were aerosolized through a Monodose RS01 high resistance DPI (Plastiape, Osnago, Italy) or a Spiriva HandiHaler. Aerosols were produced at an air flow rate of 60 L/min over four seconds for to achieve an inhalation volume of 4 L. The pressure was generated by a High Capacity Pump (model HCPS, Copley Scientific, Nottingham, UK) and controlled by a Critical Flow Controller (model TPK 2000, Copley Scientific, Nottingham, UK). NGI plates were coated with 1% glycerol in ethanol and air dried before each run. Each dry powder sample was run in triplicate. After aerosolization, dry powders deposited in the capsule, device, induction port (IP), and stages 1—MOC were dissolved in Phosphate-Buffered Saline (PBS) pH 7.4 and measured by Tecan Infinite1 200 PRO multimode microplate reader (Tecan Systems, Inc., San Jose, Calif., USA). Geometric standard deviation (GSD), mass median aerodynamic diameter (MMAD) and fine particle fraction % (FPF %) were calculated and analyzed. The FPF % was defined as the mass fraction of dry powder less than 5.0 μm or 3.0 μm with the emitted dose or metered dose.

True density. The true density was measured by the Multipycnometer (Quantachrome Instruments, Boynton Beach, Fla.) with helium as the displacement gas, which is accurate to within 0.03% of reading values.

Brunauer-Emmett-Teller (BET) Specific Surface Area (SSA) Analysis. The SSA of dry powders were analyzed by Monosorb rapid surface area analyzer model MS-21 (Quantachrome Instruments, Boynton Beach, Fla.) by single-point BET method. Samples were outgassed with nitrogen gas at 20 psi at 37° C. overnight to remove surface impurities. A mixture of nitrogen/helium (30:70 v/v) was used as the adsorbate gas.

Transfection efficiency. The transfection efficiency of the DNA plasmid (pSpCas9(BB)-2A-GFP) and LNP-mRNA was evaluated in HEK293 cells. In brief, 5×10³ of HEK293 cells were seeded in 100 μL of DMEM media in each well of 96-well plates and incubated for 24 h to allow complete adherence. After incubation, the media was removed, and Opti-MEM reduced serum media was added to the cells. 10 μL of reconstituted formulation was added to cells cultured in media with different pH 6.5. After incubation for 24 h, the transfection efficiency was evaluated by flow cytometry.

Preparation of LNP formulations. Lipid nanoparticles containing enhanced green fluorescent protein (EGFP) mRNA were prepared by combining an aqueous phase (mRNA diluted in 100 mM sodium acetate citrate buffer, pH 3.0) and an organic phase containing ethanol and lipids according to each formulation (Table 5) using a microfluidic mixer (Precision Nanosystems, Canada; Leung et al., 2015). After preparation, LNP formulations were dialyzed into 1× PBS (pH 7.4) for 2 hours in 10K MWCO Slide-A-Lyzer dialysis cassettes (Thermo Fisher Scientific, MA).

TABLE 5
Composition of LNP formulations.

| | | | Molar composition | | | |
|---|---|---|---|---|---|---|
| | | | Dlin-MC3- | | PEG- | |
| Formulation # | Phospholipid | PEG-lipid | DMA | Phospholipid | lipid | Cholesterol |
| LNP-1 | DOPE | DMPE-PEG | 0.4 | 0.2 | 0.01 | 0.39 |
| LNP-2 | DPPC | DMG-PEG | 0.6 | 0.2 | 0.01 | 0.19 |
| LNP-3 | DPPC | DMPE-PEG | 0.6 | 0.2 | 0.01 | 0.19 |
| LNP-4 | DOPE | DMG-PEG | 0.4 | 0.16 | 0.01 | 0.43 |

Statistical analysis. The statistical analysis was performed using JMP 13. All experiments were performed in triplicate. Data values are expressed as mean±standard deviations (SD). When required, Student's t-test or one-way analysis of variance (ANOVA) was performed. *p-values<0.05 were considered statistically significant.

3. Results

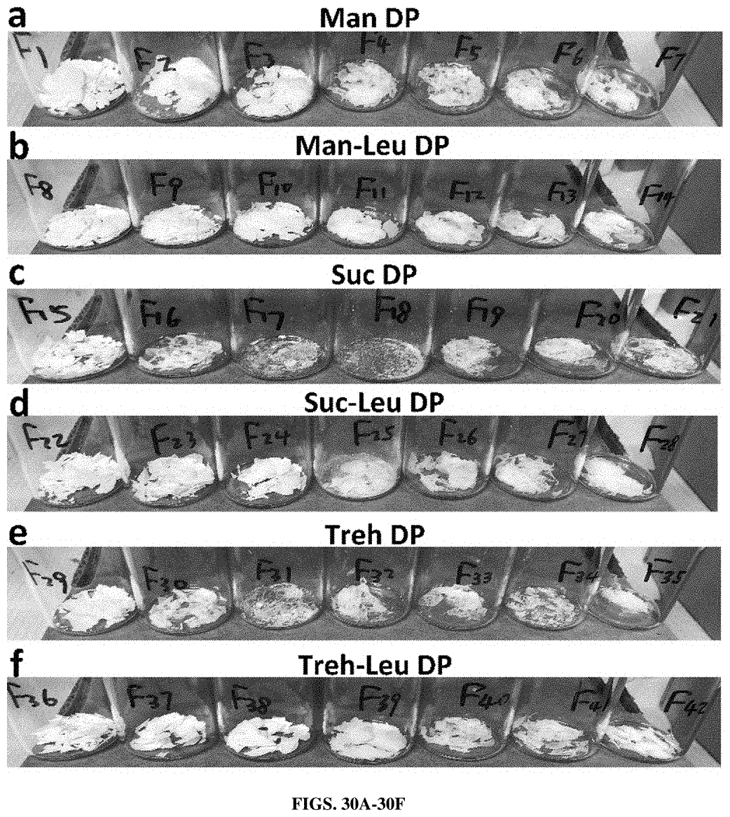

Experimental design and appearance of dry powder formulations. Three cryoprotective agents (mannitol, sucrose and trehalose) and one dispersion enhancer (leucine) were used to prepare dry powder nanocomplexes by TFF. Formulations containing seven different concentrations (10%, 5%, 3%, 1%, 0.5%, 0.25% and 0.1%; w/v) of each cryoprotective agent and PEGylated chitosan/DNA nanocomplex (50 ng/μl of DNA) were prepared with or without 0.3% of leucine in order to screen the optimal concentration for each cryoprotective agent (Table 6). Based on this experimental design, 42 formulations were prepared by TFF and the appearance of each DPI formulations were shown in FIG. 30. All formulations generally had the appearance of thin-film dry powder flakes (FIG. 30). For formulations with lower cryoprotectant amounts, the flakes were visibly smaller and/or more brittle. Formulations containing leucine were also visibly different in appearance relative to the leucine-free formulations and the thin films maintained the film structure. Specifically, for the Man DPI formulations, leucine enabled the formulation to maintain the original disk-like structure even at low mannitol concentrations (see F11-F14 versus F4-F7).

TABLE 6
Experimental design for PEGylated chitosan dry powder formulations. (DP:
dry powder, Man: mannitol, Suc: sucrose, Treh: trehalose, Leu: leucine).

| | Mannitol | Sucrose | Trehalose | Leucine | pDNA | PEGylated |
|---|---|---|---|---|---|---|
| Formulation | (w/v) | (w/v) | (w/v) | (w/v) | (ng/μl) | chitosan |
| Man DP | 10%-0.1% | — | — | — | 50 | 0.875% |
| | (F1-F7) | | | | | |
| Man-Leu DP | 10%-0.1% | — | — | 0.3% | 50 | 0.875% |
| | (F8-F14) | | | | | |
| Suc DP | — | 10%-0.1% | — | — | 50 | 0.875% |
| | | (F15-F21) | | | | |
| Suc-Leu DP | — | 10%-0.1% | — | 0.3% | 50 | 0.875% |
| | | (F22-F28) | | | | |
| Treh DP | — | — | 10%-0.1% | — | 50 | 0.875% |
| | | | (F29-F35) | | | |
| Treh-Leu DP | — | — | 10%-0.1% | 0.3% | 50 | 0.875% |
| | | | (F36-F42) | | | |

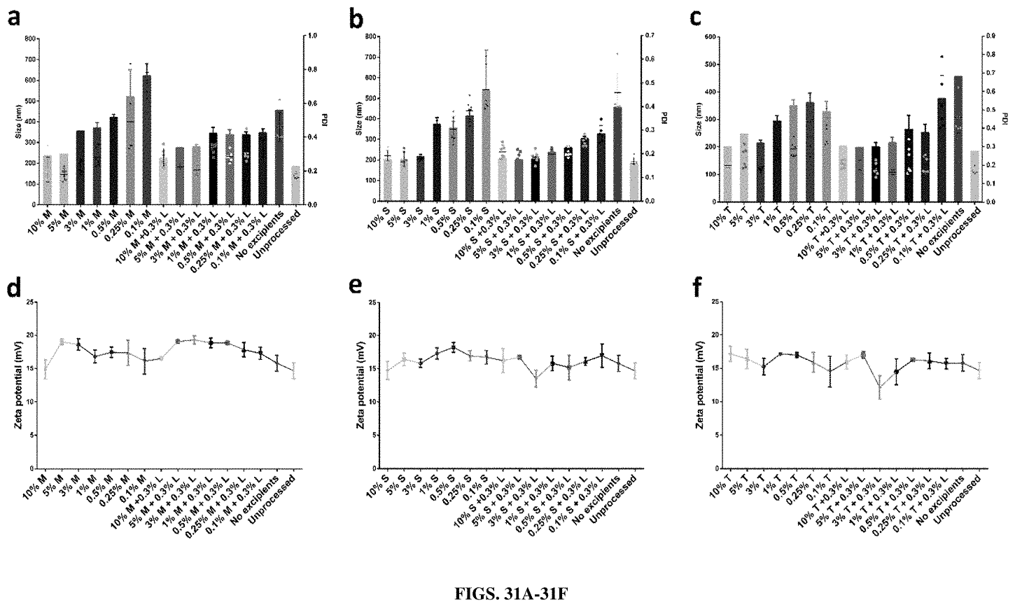

Size and zeta potential of nanocomplexes after reconstitution. The size and zeta potential were measured by zetasizer to assess size changes after processing and reconstitution to evaluate nanocomplex physical stability during manufacture. As shown in FIGS. 31A-31C, every reconstituted formulation had a statistically significant increase in particle size compared to the nanocomplex without TFF processing (184.1±6.6 nm). The size of reconstituted Man-DP formulations ranged from 235.0±39.2 nm (F1) to 621.2±58.3 nm (F7), while the size of reconstituted Man-Leu DP formulations ranged from 223.4±30.2 nm (F8) to 345.7±20.1 nm (F14). For Suc DP formulations, the particle size ranged from 200.4±9.2 nm (F15) to 536.0±198.8 nm (F21), while the particle size of Suc-Leu DP ranged from 206.8±11.1 nm (F22) to 326.4±21.6 nm (F28). For the Treh DP formulations, the smallest particle size was observed in F29 and the formulation showed largest particle size is F35. With the addition of leucine (Treh-Leu DP), the particle size ranged from 202.9±4.5 nm (F36) to 376.3±47.6 nm (F42). In sum, a trend of an increasing nanocomplex size was observed with a decreasing concentration of cryoprotective agents. In contrast, no obvious trend was observed in terms of the zeta potential of DP formulations.

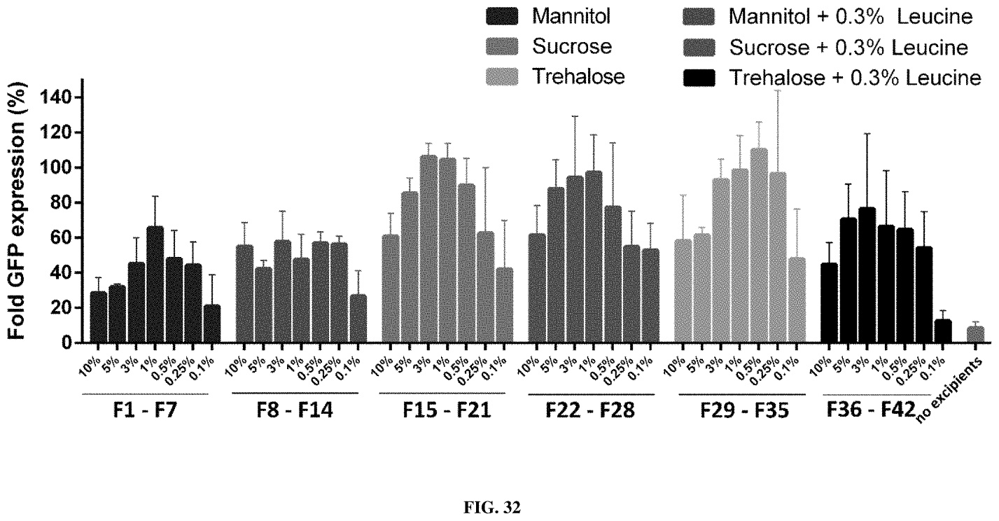

Transfection efficiency of nanocomplexes after reconstitution. The effects of type and concentration of cryoprotective agents on the transfection efficiency of nanocomplex was tested. FIG. 32 showed the transfection efficiency of the reconstituted formulations data normalized to the unprocessed nanocomplexes. It was found that the either high or low concentration of the cryoprotective agent was not able to protect the potency of nanocomplexes from the TFF/lyophilization or reconstitution steps. The highest transfection efficiency was observed in formulations containing 1% of mannitol, 3% of sucrose, 0.5% of trehalose, 3% of mannitol+0.3% of leucine, 1% of sucrose+leucine, and 3% of Trehalose+0.3% of leucine for Man DP, Suc DP, Treh DP, Man-Leu DP, Suc-Leu DP, and Treh-Leu DP, respectively. In contrast, the nanocomplexes without any excipients showed little transfection efficiency after TFF and lyophilization process.

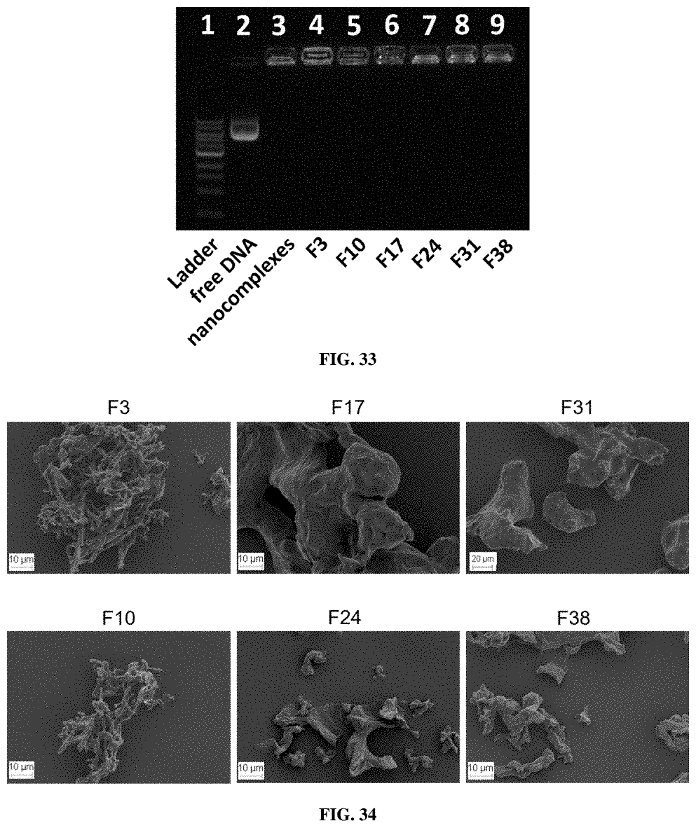

Based on these screening assays, it was found that a higher concentration of cryoprotective agent resulted in less aggregation of the nanocomplexes after reconstitution (i.e. lower particle size changes) however the highest transfection efficiency was found with formulations containing cryoprotectant concentrations ranging from 0.5-3%. Thus, six formulations (F3, F10, F17, F24, F31, and F38) containing 3% of cryoprotective agents were selected as lead formulations for further investigation (FIG. 33).

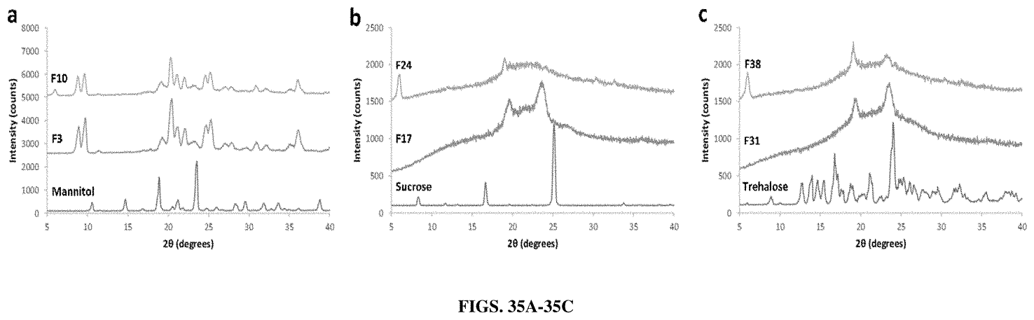

Characterization of lead dry powder formulations. SEM images (FIG. 34) revealed that all six of the dry powder formulations exhibited aggregation to different extents, among which, F3 and F10 demonstrated greater porosity compared to the other four formulations which exhibited smooth solid in appearance. These observations were combined with the particle size of the powders. The geometric particle size distribution of six lead dry powder formulations were characterized by HELOS laser diffraction using RODOS powder dispersion. As shown in Table 7, it appeared that the median geometric particle size (D50) of F3 and F10 were significantly smaller than the other formulations. Given the appearance and lower porosity of formulations F17, F24, F31, F38 with their larger D50 they are likely not respirable powders. This was confirmed by the aerodynamic particle sizing described above. X-ray diffractograms revealed that the unprocessed raw mannitol exhibited the β form with the characteristic diffraction peaks at 2θ of 10.54° and 14.69° while the TFF dry powder formulations (F3 and F10) demonstrated a δ form as a characteristic diffraction peak at 2θ of 9.69° and no diffraction peak from 10° to 16° were observed (FIG. 35A). As shown in FIGS. 6b and 6c, F17, F24, F31, and F38 appeared amorphous as no obvious crystalline peaks were observed compared to the unprocessed sucrose (FIG. 35B) and trehalose (FIG. 35C).

TABLE 7
Average geometric particle size distribution
of refined dry powder formulations.

| | D₁₀ | D₅₀ | D₉₀ | |
|---|---|---|---|---|
| F # | (μm) | (μm) | (μm) | span |
| F3 | 1.1 ± 0.1 | 3.4 ± 0.3 | 8.9 ± 1.5 | 2.3 |
| F10 | 1.1 ± 0.02 | 3.4 ± 0.3 | 9 ± 1.5 | 2.3 |
| F17 | 4.7 ± 0.7 | 42.5 ± 3.2 | 113.2 ± 9.3 | 2.6 |
| F24 | 2.1 ± 0.2 | 9.7 ± 1.0 | 24.4 ± 5.5 | 2.3 |
| F31 | 6.2 ± 2.8 | 27.7 ± 8.8 | 72.3 ± 16.2 | 2.5 |
| F38 | 2.0 ± 0.5 | 8.1 ± 2.7 | 21.1 ± 6.5 | 2.4 |

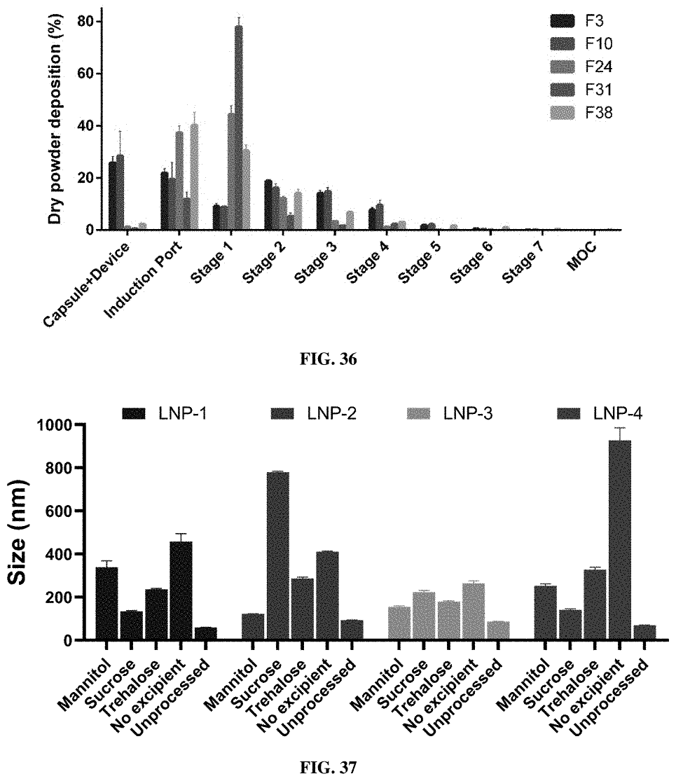

Aerodynamic performance of refined dry powder formulations in RS01 Monodose DPI. NGI was used to evaluate the aerodynamic performance of refined dry powder formulations which were aerosolized by the low resistance RSO1 Monodose DPI (flow rate 60 L/min). As shown in FIG. 36, F3 and F10 rendered a higher deposition below stage 2 (4.46 microns aerodynamic cutoff) compared to other formulations, which indicated a better aerodynamic particle size distribution of F3 and F10. Based on the deposition profile, MMAD, FPF %, and EF % were calculated and summarized in Table 8. F3 and F10 demonstrated a MMAD of 4.8 μm and 4.6 μm, respectively, which indicated a better potential for dry powder particle deposition in lung compared to other formulations which had MMADs larger than 5 μm. Furthermore, even though the EF % of F3 (74.2%) and F7 (71.5%) were lower than that of other formulations, F3 and F10 demonstrated a relatively higher FPF % (<5 μm) of 44.5% and 44.2% than other formulations, respectively. Based on these results, F3 and F10 were identified as the formulations suitable for inhalation and were tested further.

TABLE 8
Aerodynamic performance of refined TFF dry
powder formulations in RS01 at 60 L/min.

| | Flow | | | | | |
|---|---|---|---|---|---|---|
| | rate | | FPF % (<5 μm) | FPF % (<5 μm) | MMAD | |
| F # | (L/mtn) | EF % | (of emitted) | (of metered) | (μm) | GSD |
| F3 | 60 | 74.2 ± 2.5 | 60 ± 2.2 | 44.5 ± 2.5 | 4.8 ± 0.3 | 1.3 |
| F10 | 60 | 71.5 ± 9.4 | 62.3 ± 4.4 | 44.2 ± 3.0 | 4.6 ± 0.4 | 1.3 |
| F17 | 60 | — | — | — | — | — |
| F24 | 60 | 98.8 ± 0.2 | 24 ± 0.6 | 23.7 ± 0.5 | N/A | N/A |
| F31 | 60 | 99.4 ± 0.2 | 21.3 ± 0.6 | 21.2 ± 0.6 | N/A | N/A |
| F38 | 60 | 97.7 ± 0.3 | 32.4 ± 3.2 | 31.7 ± 3 | N/A | N/A |

Effects of type of inhaler and flow rate on the aerodynamic performance of F3 and F10. To further evaluate the aerodynamic performance of F3 and F10, two types of high resistance inhalers were used to aerosolize dry powder formulations at two different flow rates (Table 9). It appeared that F10, which contains leucine, showed a lower MMAD and a higher FPF % compared to F3 irrespective of inhaler type or flow rate. In addition, for either HandiHaler or RS01 DPI, both formulations had a significant higher EF % and FPF %, and a lower MMAD at the flow rate of 60 L/min compared to that at the flow rate of 45 L/min, which indicated a flow rate-dependent aerodynamic performance of F3 and F10 in these devices. Furthermore, at the same flow rate, HandiHaler DPI rendered a higher EF %, but a larger MMAD, for either F3 or F10 compared to that of RS01 Monodose DPI, which indicated that the aerodynamic performance of both formulations was also inhaler type dependent.

TABLE 9
Aerodynamic performance of F3 and F10 in different inhalers and at different flow rates. (N/A indicated the size was out of measurement range).

| | | Flow rate | Pressure | | FPF %(<5 μm) | FPF %(<5 μm) | FPF %(<3 μm) | FPF %(<3 μm) | MMAD | |
|---|---|---|---|---|---|---|---|---|---|---|
| inhaler | F # | (l/min) | drop | EF % | (emitted) | (metered) | (emitted) | (metered) | (μm) | GSD |
| RSO1 | F3 | 60 | 4.1 kPa | 74.2 ± 2.5 | 60 ± 2.2 | 44.5 ± 2.5 | 35.6 ± 2.5 | 26.4 ± 2.4 | 4.8 ± 0.3 | 1.3 |
| | F10 | 60 | 4.1 kPa | 71.5 ± 9.4 | 62.3 ± 4.4 | 44.2 ± 3.0 | 40.0 ± 0.9 | 27.7 ± 5.6 | 4.6 ± 0.4 | 1.3 |
| HandiHaler | F3 | 60 | 8.1 kPa | 98.7 ± 0.5 | 43.3 ± 4.6 | 42.7 ± 4.8 | 23.5 ± 2.5 | 23.2 ± 2.5 | N/A | N/A |
| | F10 | 60 | 8.1 kPa | 98 ± 0.7 | 63 ± 4.9 | 61.7 ± 4.4 | 40.3 ± 5.9 | 39.4 ± 5.5 | 4.9 ± 0.9 | 1.4 |
| RSO1 | F3 | 45 | 2.3 kPa | 29.9 ± 4.7 | 23 ± 5.4 | 7.1 ± 2.7 | 11.8 ± 3.5 | 3.6 ± 1.6 | N/A | N/A |
| | F10 | 45 | 2.3 kPa | 39.2 ± 4 | 39 ± 3.6 | 15.4 ± 2.7 | 24.1 ± 2.8 | 9.5 ± 1.9 | 7 ± 0.9 | 1.3 |
| HandiHaler | F3 | 45 | 4.2 kPa | 85.9 ± 5.3 | 18.4 ± 5.1 | 16 ± 5.4 | 9.6 ± 3.9 | 8.4 ± 3.9 | N/A | N/A |
| | F10 | 45 | 4.2 kPa | 90.6 ± 1.7 | 24 ± 3.2 | 21.8 ± 2.9 | 13.1 ± 1.5 | 11.8 ± 1.4 | N/A | N/A |

The moisture content, true density and specific surface area of F3 and F10 were evaluated by TGA, multipycnometer, and monosorb (rapid surface area analyzer BET). As shown in Table 10, F10 containing leucine had a lower moisture content and lower true density compared to that of F3. In contrast, the specific surface area of F10 was significantly higher than F3.

TABLE 10
True density and specific surface area of F3 and F10.

| | True density | Specific surface |
|---|---|---|
| Formulation | (g/cm³) | area (m²/g) |
| F3 | 1.681 ± 0.077 | 3.13 ± 0.12 |
| F10 | 1.554 ± 0.037 | 3.80 ± 0.14 |

Size of TFF lipid nanoparticle-mRNA (LNP) dry powder formulations. Four LNP formulations consisting of ionizable lipids, phospholipids, cholesterol, poly-(ethylene) glycol (PEG)-lipid), and mRNA encoding EGFP were formulated into dry powder by TFF with different excipients at a concentration of 20% (w/w): mannitol, sucrose, and trehalose were employed. After TFF and lyophilization, the dry powder formulations were reconstituted in distilled water and the LNP particle size were measured by DLS. As shown in FIG. 37, although all TFF formulations after reconstitution showed a significant increase in particle size compared to unprocessed LNP, different cryoprotective agents showed different cryoprotective effects on each formulation. For LNP-1 and LNP-4, sucrose showed a better protective effect on size than mannitol and trehalose due to the least size change after reconstitution, while for LNP-2 and LNP-3, mannitol showed a better protective effect on size than sucrose and trehalose.

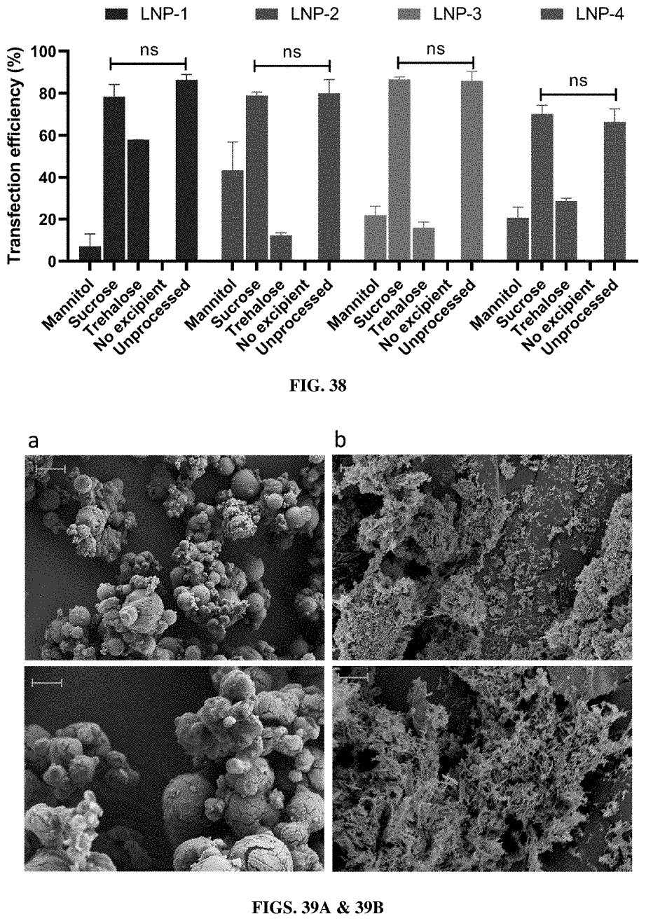

Intracellular uptake of TFF lipid nanoparticle (mRNA loaded) dry powder formulations. The transfection efficiency of dry powder LNP formulations after reconstitution was evaluated in HEK293 cells. As shown in FIG. 38, formulations with 20% of sucrose showed no significant difference in transfection efficiency compared to the unprocessed LNP formulations, while other cryoprotective agents showed a significant decrease in transfection efficiency compared to the unprocessed LNP formulations.

### Example 4—Thin-film Freeze-Dried siRNA-Encapsulated Solid Lipid Nanoparticles for Potential Pulmonary Delivery

### A. Material and Methods

1. Materials

Polyethylene glycol 2000-hydrazone-C18 (PHC) was synthesized following previously published methods and characterized by NMR (Zhu et al., 2013). Refined lecithin was obtained from Alfa Aesar (Tewksbury, Mass.). Mannitol (USP), lipopolysaccharides (LPS), cholesterol, Type III Mucin, Amicon Ultra centrifugal filter units Ultra-15 (MWCO 100 kDa) from the porcine stomach from Sigma-Aldrich (St. Louis, Mo., USA). Lipofectamine RNAiMAX Transfection Reagent, Dulbecco's Modified Eagle Medium (DMEM), fetal bovine serum (FBS), streptomycin/penicillin, FluoSpheres™ amine-modified polystyrene microspheres, and HEPES buffer were from Invitrogen (Carlsbad, Calif.). TopFluor® Cholesterol and 1,2-dioleoyl-3-trimethylammonium-propane (DOTAP) were from Avanti Polar Lipids (Alabaster, Ala., USA). TNF-α siRNA was purchased from Integrated DNA Technologies (Coralville, Iowa, USA) with sequence (5′-GUCUCAGCCUCUUCUCAUUCCUGCT-3′ (SEQ ID NO: 1), anti-sense: 5′-AGCAGGAAUGAGAAGAGGCUGAGACAU-3′ (SEQ ID NO: 2)) TNF-α ELISA kit was from BioLegend (San Diego, Calif.).

2. Methods

Preparation of nanoparticles suspension. SLNs were prepared followed a previously established solvent evaporation method with slight modifications (Aldayel et al., 2018). Briefly, lecithin (3.2 mg), cholesterol (1.6 mg), PHC (2 mg), and 8 μL of TopFluor cholesterol solution (0.25% w/v in THF) were dissolved in 0.5 mL of THF and filtered through 0.2 μm PTFE syringe filter. The mixture was added dropwise to 5 mL of water with stirring. The resultant nanoparticles suspension was stirred overnight to evaporate THF, then filtered with a 3.2 μm PTFE syringe filter and then stored at 4° C. before drying procedures.

To prepare siRNA incorporated SLNs, 100 μl of 20 μM siRNA in water was diluted with 400 μL of water and then added to 680 μL of 2.56% (v/v) DOTAP in chloroform and stirred vigorously for 30 min, followed by the addition of 1.3 mL of methanol stirred for 1 h. The siRNA/DOTAP complexes were extracted with chloroform from the mixture by phase separation. Lecithin (3.2 mg), cholesterol (1.6 mg), and PHC (2 mg) were dissolved in 0.5 mL of chloroform and mixed with the siRNA/DOTAP complexes. The mixture was dried under nitrogen gas and then re-dissolved in 500 μL of THF before adding dropwise to 5 mL of water. To fluorescently label the SLNs, a TopFluor cholesterol solution (0.25% w/v in chloroform) was added to the lecithin mixture before mixing with the siRNA/DOTAP complexes. The size, polydispersity index (PDI), and zeta potential of the resultant SLNs were measured by dynamic light scattering (DLS) using a Malvern Zeta Sizer Nano ZS (Westborough, Mass.).

Dry powder preparation by thin-film freeze-drying. To prepare thin-film freeze-dried SLNs powder, mannitol was dissolved in the nanoparticle suspension (40.8 mg/mL for SLNs siRNA-free SLNs and 48 mg/mL for siRNA-encapsulated SLNs) and then frozen by dropping the suspension to a rotation, pre-cooled, hollow stainless steel cylindrical drum as previously described (Zhang et al., 2012; Engstrom et al., 2008; Thakkar et al., 2017). Lyophilization cycle was −40° C. shelf temperature for 20 h, ramped to 25° C. over 20 h, then hold at 25° C. for another 20 h with pressure blow 100 mPa using a VirTis AdVantage Bench Top Lyophilizer (Gardiner, N.Y., USA). The mannitol to SLNs ratio was determined by a freeze-and-thaw experiment. Briefly, 1 mL of the SLNs in suspension were mixed with different amount of mannitol and froze at −80° C. for 2 h and then thawed at room temperature before measuring the particle size and PDI.

Dry powder preparation by spray drying. The spray-dried nanoparticle powder was prepared by dissolving mannitol into the nanoparticle suspension at 4.08 mg/mL, which was then dried using a Büchi B-290 Mini Spray Dryer (Flawil, Switzerland) with a ø0.5 mm two-fluid nozzle. The flow of the aerosolization gas was 29 L/min (nitrogen), the aspirator was set to 100 psi, the inlet temperature was 90° C. and the outlet temperature was 65° C., and the suspension feed rate was 3 mL/min. The powder was stored in a vacuum desiccator in dark until analysis. The powder was stored in a vacuum desiccator in dark until further analysis.

Powder characterizations. The morphology of spray-dried powder and TFFD powder was examined using Zeiss Supra 40V Scanning Electron Microscope (SEM) (Zeiss SMT AG, Oberkochen, Germany) in the Microscopy and Imaging Facility at the University of Texas at Austin (Austin, Tex.). Samples were tapped to the conductive tape and then coated with 15 nm Au/Pt with a Cressington 208 sputter coater (Cressington Scientific Instruments, Watford, UK) before loading to the SEM. Surface areas of dried powders were measured with a Quantachrome Nova 2000 Brunauer-Emmett-Teller (BET) surface area analyzer (Quantachrome Corporation, Boynton Beach, Fla.).

In vitro aerosolization properties. The in vitro aerosol performance of the powders was evaluated with a Next Generation Impactor (NGI) and CITDAS software from Copley Scientific (Colwick, Nottingham, UK). Approximately 10 mg dry powder was filled into a size 3 Hydroxypropyl methylcellulose (HPMC) capsule (Capsugel, Morristown, N.J.) and placed into the RS01 high resistance inhaler from Plastiape (Milano, Italy). Fine particle fraction (FPF) was defined as an aerodynamic diameter <5 microns. Quantification of the SLNs was achieved by measuring the fluorescence intensity of the NPs using a BioTek Synergy HT Microplate Reader (ex=485, em=528) (Winooski, Vt.) with the following equation: % deposition=100×(fluorescence intensity of the resuspended powder on each NGI stage/volume of resuspension media)/(fluorescence intensity of the resuspended powder standard/volume of resuspension media). An ethanol in water solution (50%, v/v) was chosen as the resuspension media since the fluorescence signal was relatively weak in pure water.

Nanoparticle diffusion in stimulated mucus. The diffusion of SLNs and polystyrene beads were compared in simulated mucus was measured using a previously developed assay (Leal et al., 2018). Mucin was dissolved in 20 mM HEPES buffer to make 2% (w/v) solution and gently agitated for 30 min, then 100 μL of the simulated mucus was transferred to the top compartment in the polyester membrane Corning® Transwell insert with 3.0 μm pore size (Corning, N.Y.) against 600 μL of 20 mM HEPES buffer in the bottom compartment, and the Transwells were left at room temperature. The pore size was selected to ensure the particle can move through the membrane while retain the mucin gel during the time course of the experiment (Norris and Sinko, 1997). Next, 10 μl of reconstituted SLNs or polystyrene beads (as a control) were gently added to the top compartment. The bottom HEPES buffer was collected and replaced with fresh HEPES buffer every hour for 5 h. Wells without mucin gel was used as a control. The particle amount in collected eluates was determined from the fluorescence intensity based on a 6 points linear calibration curve.

ELISA. TNF-α SLN powder (100 mg) was resuspended in 5 mL of serum-free media and then filtered with 3.2 μm PTFE filter. J774A.1 macrophage cells (American Type Culture Collection, Manassas, Va.) were seeded in a 96-well plate (7000 cells/well). After overnight incubation, the medium was replaced by 150 μL/well of the suspension. After 4 h, 150 μL of media with 20% FBS was added, and the cells incubated for forty-four (44) additional hours. The medium was then replaced with 300 μL/well of medium containing LPS at 300 ng/mL and incubated for 4 h before measuring the TNF-α concentration by a BioLegend ELISA kit.

Statistical analysis. Diffusion and ELISA data were processed with Prism (GraphPad Software, San Diego, Calif.).

### B. Results and Discussion

TFFD is a fast-freezing process followed by lyophilization. Dry powder prepared by TFFD is porous with a high surface area. The method has been successfully applied to small molecules (Zhang et al., 2012; Overhoff et al., 2008; Overhoff et al., 2007), proteins (Engstrom et al., 2008), and vaccines adjuvanted with insoluble aluminum salts (Thakkar et al., 2017; Li et al., 2015). In addition, the fluffiness and brittleness of the powder give it excellent aerosol properties for pulmonary drug delivery. Pulmonary delivery of small molecules (Patlolla et al., 2010; Nemati et al., 2019; Patil-Gadhe et al., 2016) and nucleic acid-based agents (Hyde et al., 2014; Deshpande et al., 2002) has proven feasible using lipid-based particles as carriers. Both spray drying (Nemati et al., 2019) and freeze-drying (Lball et al., 2017) have been used to prepare dry powder formulation of SLNs. However, it was reported that the aerodynamic properties of ethambutol-loaded SLN dry powder prepared by spray drying were not favorable for deep lung delivery, due to its large particle size (Nemati et al., 2019). Since only particles with the size between 1 μm to 5 μm can be deposited to the deep lung, SLNs with diameters in the range of 100-200 nm are too small and will be exhaled after inhalation (Rahimpour and Hamishehkar, 2012). Therefore, SLNs require excipient(s) to act as a carrier and cytoprotectant(s) for dry powder formation. In this study, the feasibility of applying TFFD to SLNs for pulmonary delivery was tested. The SLNs were prepared by the solvent evaporation method as previously described (Aldayel et al., 2018). They were prepared with lecithin, cholesterol, and PHC, with or without siRNA complexed with a cationic lipid. The resultant SLNs were approximately 100-150 nm in diameter (measured by DLS), relatively uniformly distributed, and spherical. The siRNA-free SLNs were then subjected to TFFD or spray-drying and the powders generated were compared. SLNs encapsulated with TNF-α siRNA were then subjected to TFFD. The dry powder of the TNF-α siRNA-SLNs was characterized, its aerosol properties measured, as well as the function of the TNF-α siRNA-SLNs after they were subjected to TFFD and reconstitution and the ability of the TNF-α siRNA-SLNs to permeate through simulated lung mucus.

Screening excipients to freeze SLNs. Before the SLNs were subjected to TFFD, potential cryoprotectants were screened for their ability to protect the SLNs during freezing. Mannitol was selected as the powder bulking agent and cryoprotectant because of its good aerosol performance property (D'Addio et al., 2013) and cryoprotection ability (Wang et al., 2018). To determine the SLNs to mannitol ratio needed, a freeze-and-thaw experiment was performed. Results in Table 11 showed that the best cryoprotection was achieved with the particle to a mannitol weight ratio of 1 to 30, which was then used for further studies.

TABLE 11
Freeze-and-thaw of SLNs in the presence of various amounts
of mannitol. Data are mean ± SD (n = 3).

| | Before freeze | After freeze-and-thaw | | |
|---|---|---|---|---|
| | Particle to mannitol ratio | | | |
| | | 1:10 | 1:20 | 1:30 |
| Particle size (nm) | 129.2 ± 5.7 | 329.7 ± 55.9 | 174.3 ± 11.7 | 140.2 ± 7.9 |
| PDI | 0.239 ± 0.014 | 0.582 ± 0.070 | 0.336 ± 0.052 | 0.288 ± 0.052 |

Preparation and characterization of thin-film freeze-dried powder of SLNs. Dry power of SLNs was prepared by dropping SLNs suspended in mannitol solution to a pre-cooled metal surface and lyophilized in a shelf-freeze dryer. As a control, a spray dried powder of the SLNs was also prepared with the same composition. The TFFD powder and spray dried (SD) powder of SLNs were first characterized by measuring the particle size, PDI, and zeta potential after reconstitution. As shown in Table 12, the size of the SLNs reconstituted from the SD and TFFD powders increased, as compared to the SLNs before drying. The PDI of the SLNs did not change after they were subjected to TFFD and reconstitution, although it was increased after subjected to SD and reconstitution. The mechanism underlining the increase in particle size is not known, but freezing stress (Chung et al., 2012) as well as stress during the drying step and particle excipient interaction may have contributed to the particle size increase (Niu and Panyam, 2017). The powders were then characterized by examining their morphology and specific surface area. As shown in FIG. 39, the TFFD powder demonstrated porous texture, while the SD powder showed beads-like microstructures. The specific surface area of TFFD powder is approximately 20 times higher than the SD powder (Table 12), which was consistent with previous literature (Engstrom et al., 2008).

TABLE 12
Physical properties of the SLNs before and after
they were subjected to spray drying or TFFD and
reconstitution. Data are mean ± SD (n = 3).

| | Before | Spray | |
|---|---|---|---|
| | drying | drying | TFFD |
| Particle size (nm) | 110.9 ± 0.73 | 141.8 ± 1.64 | 162.2 + 1.14 |
| PDI | 0.2 ± 0.01 | 0.4 ± 0.01 | 0.2 ± 0.02 |
| Zeta potential (mV) | −34 ± 0.01 | −35.2 ± 1.39 | −40.6 ± 0.26 |
| Specific surface | N/A | 0.92 ± 0.12 | 19.34 ± 2.57 |
| area (m²/g) | | | |

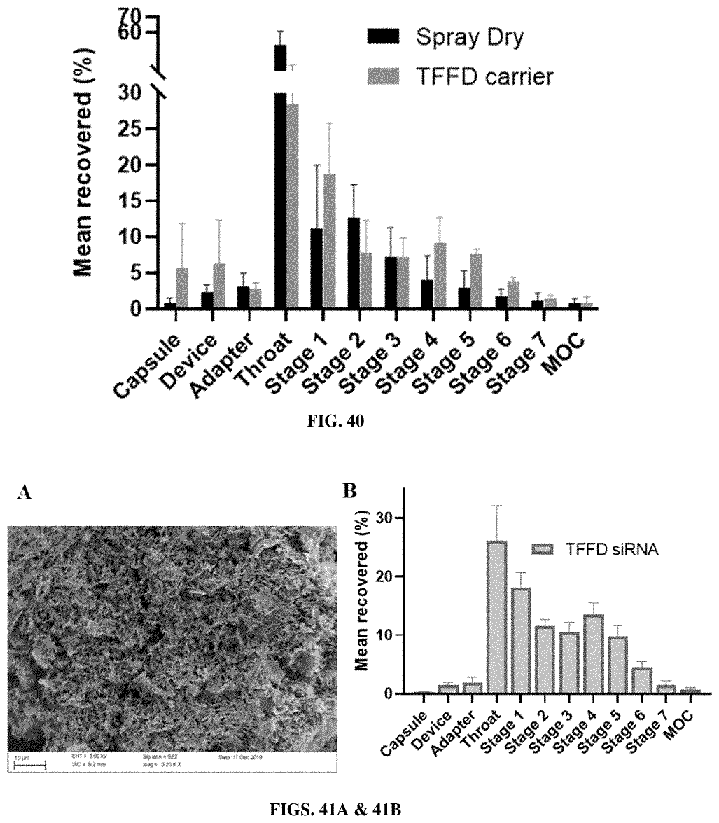

In vitro aerosolization performance. The aerosol performances of the SD powder and TFFD powder were determined and compared using NGI (FIG. 40). The TFFD powder demonstrated a higher FPF (Table 13) and better deposition in the deep lung area than the SD powder formulation (FIG. 40, see stages 4-7), likely due to the porous morphology and high surface area of the TFFD powder. Therefore, it was concluded that at the composition tested, the SLNs dry powder prepared by TFFD was better than that prepared by spray drying for pulmonary delivery of the SLNs.

TABLE 13
The fine particle fraction (FPF) %, mass median aerodynamic
diameter (MMAD), and geometric standard deviation
(GSD) values of dry powder of SLNs prepared by spray
drying or TFFD (data are mean ± SD, n ≥ 3)

| | Spray dried | TFFD powder |
|---|---|---|
| | powder of SLNs | of SLNs |
| FPF % | 22.42 ± 12.88 | 37.01 ± 4.52 |
| MMAD | 5.97 ± 1.73 | 3.96 ± 0.97 |
| GSD | 2.30 ± 0.49 | 3.25 ± 0.61 |

Preparation and characterization of thin-film freeze-dried powder of siRNA-encapsulated SLNs. To prepare siRNA-encapsulated SLNs, siRNA was mixed with a biocompatible cationic lipid, DOTAP, at a N to P ratio of 12 to 1 and then mixed with other ingredients followed by solvent evaporation as previously described. The resultant siRNA-SLNs had a slightly larger particle size compared to the siRNA-free SLNs (Table 14). The siRNA-SLNs in suspension were mixed with mannitol at ratio of 1:30, w/w, and subjected to TFFD. The powder as shown in FIG. 41A were fluffy with porous texture. The size of the siRNA-SLNs increased slightly after they were subjected to TFFD and reconstitution. FIG. 41B showed the aerosol performance characteristics of the siRNA-SLN powder prepared by TFFD. Again, the siRNA-SLN powder had a high FPF % (Table 15), and high deposition in stages representing the deep lung (FIG. 41B). The main factor for delivery to alveoli of the lung is the aerodynamic particle size. Thin-film freeze-dried siRNA-SLN powder demonstrated smaller MMAD, higher FPF %, and higher deposition to the NGI stages corresponding to alveoli than previously published methods (Nemati et al., 2019; Ohashi et al., 2009), suggesting that TFFD is ideal for generating dry power of siRNA-SLNs for aerosol delivery.

TABLE 14
A comparison of the physical properties of TNF-a siRNA-
SLNs before and after they were subjected to TFFD and
reconstitution. Data are mean ± SD (n = 3).

| | Before | After |
|---|---|---|
| | drying | TFFD |
| Particle size | 146.9 ± 0.78 | 193.4 ± 5.2 |
| PDI | 0.2 ± 0.01 | 0.3 ± 0.03 |
| Zeta potential (mV) | −39.6 ± 3.16 | −33.3 ± 0.03 |

TABLE 15
The fine particle fraction (FPF) %, mass median
aerodynamic diameter (MMAD), and geometric standard
deviation (GSD) values of dry powder of siRNA-SLNs
prepared by TFFD (data are ±mean SD, n ≥ 3).

| | TFFD powder of |
|---|---|
| | siRNA-SLNs |
| FPF % | 44.48 ± 5.65 |
| MMAD | 3.60 ± 0.43 |
| GSD | 2.81 ± 0.16 |

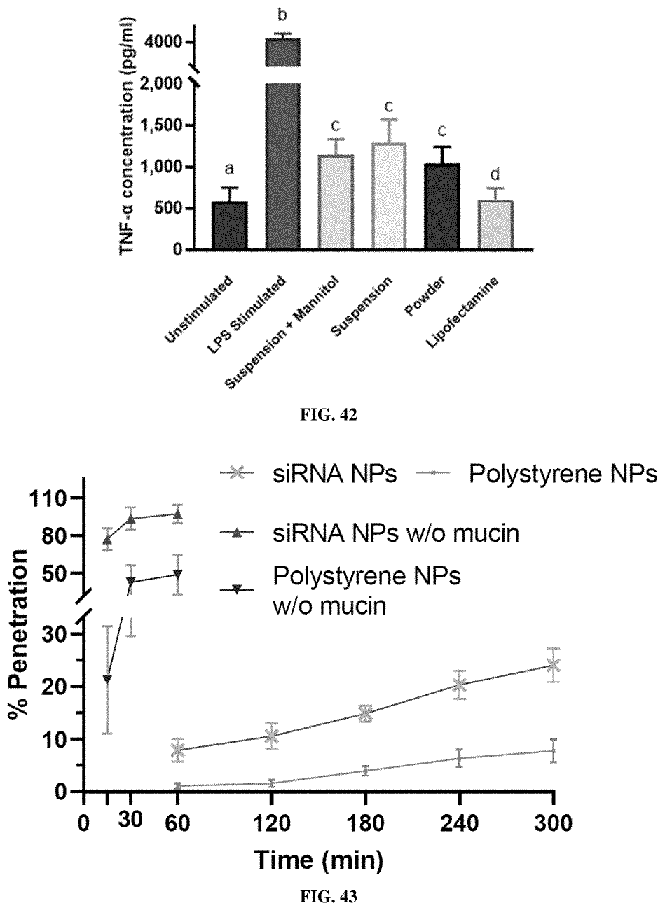

Verification of the function of siRNA in the siRNA-SLNs after they were subjected to TFFD and reconstitution. To verify the function of the siRNA after the siRNA-SLNs were subjected to TFFD and reconstitution, TNF-α siRNA-SLNs were used, and the siRNA-SLNs' ability to suppress the expression of TNF-α by J774A.1 mouse macrophages stimulated with LPS was measured. As shown in FIG. 42, the TNF-α siRNA-SLNs after subjected to TFFD and reconstitution were as effective as those before TFFD in downregulating TNF-α release from the cells, demonstrating that TFFD can be successfully applied to transform the siRNA-SLNs from a liquid suspension to dry powder without compromising the functionality of the siRNA.

Diffusion of the siRNA-SLNs across simulated mucus. For siRNA-SLNs delivered to the lung to have access to live cells, they need to permeate through the mucus layer. To evaluate if siRNA-SLNs can permeate through the mucus layer after delivery to the lung, a mucus penetration assay was performed using a system consist of a Transwell permeable support with or without a simulated mucus (Norris and Sinko, 1997; Desai et al., 1991). The SLNs in suspension were added gently on the mucus, in the center of the well without disturbing the mucus, and the particle concentration in the other side of the Transwell was quantified at different time points. Commercially available fluorescently labeled polystyrene beads (size, 279±4 nm; PDI, 0.10±0.02; Zeta potential, +36.0±0.4 mV) were used as a control. Shown in FIG. 43 are the percentages of particles diffused through the membrane with or without the simulated mucus layer. Without the simulated mucus, both SLNs and polystyrene beads diffused through the membrane rapidly and reached the plateau within one hour. The diffusion of the siRNA-SLNs across the mucus layer was clearly slower (FIG. 43), but about 25% of the SLNs diffused through the simulated mucus layer within 5 h, clearly indicating that the siRNA-SLNs can permeate the mucus in the lung after they are aerosolized into the lung as thin-film freeze-dried powder.

In the present study, it was demonstrated to be feasible to produce a dry powder formulation of SLNs with good aerosol properties for potential pulmonary delivery of a therapeutic agent such as TNF-α siRNA into the lung. Initially, dry powder of SLNs were prepared by spray drying and TFFD and compared their physical and aerosol properties. The powder prepared by TFFD were fluffy and brittle, demonstrated better aerosol properties than the spray-dried powder. It was further shown that the TFFD powder of TNF-α siRNA-encapsulated SLNs remained functional in their ability to downregulate TNF-α release by macrophages in culture. With their demonstrated ability to penetrate simulated mucus, likely due to surface-PEGylation of the nanoparticles (Huckaby and Lai, 2018), it is expected that the TFFD powder of siRNA-SLNs can be potentially used for pulmonary delivery of the siRNA to the lung to treat pulmonary diseases, such as asthma and other chronic inflammatory diseases, using siRNA specific to key proinflammatory cytokines such as TNF-α. Of course, the siRNA does not have to be TNF-α siRNA, and in fact, it is expected that other nucleic acid-based agents, such as mRNA, shRNA, plasmid DNA, minicircle DNA, DNA oligos, may also be formulated into the SLNs or lipid nanoparticles similar to the SLNs used in this study. In addition, the nanoparticles do not need to be lipid-based; nanoparticles of polymer-based or made of inorganic nanoparticles may also be converted from a liquid suspension to dry powder using TFFD for aerosolization. Furthermore, nanoparticles are commonly used as carriers to protect nucleic acid-based agents and to improve their uptake by target cells. If the nucleic acid-based agents are specially engineered to be stable and/or can be taken up by target cells without the help of the nanoparticles, then they can be directly converted into dry powder with good aerosol properties using TFFD. Moreover, it is obvious that the therapeutic and/or diagnostic agents encapsulated into the nanoparticles do not have to be nucleic acid-based. Small molecules, proteins, and even bacteria and viruses may be carried by the nanoparticles. Finally, any potential therapeutic and diagnostic agents may also be mixed with nanoparticles before they are subjected to TFFD.

Freeze drying of colloidal suspension has been described in detail before, and it was shown that the increase in the size of the colloidal nanoparticles caused by the bulking agent is universal in stable colloidal systems (Lintingre et al., 2016). This may explain the increase in the hydrodynamic diameters of the SLNs, encapsulated with siRNA or not, after they were subjected to TFFD. The ratio of SLNs to excipients plays a significant role in affecting the particle size and polydisperse index (PDI) of the SLNs. Freeze drying of colloidal suspension is a multiple-step process, and it is rather difficult to describe such a process. In the freezing step, the particle aggregation caused by freezing is mainly attributed to ice crystallization, which pushes particles to a small area with high freezing stress. In the drying step, the excipient(s) serve as a water surrogate, stabilizing the particles by establishing hydrogen bonds with the particle surface (Abdelwahed et al., 2006). TFFD technology is unique in two aspects: First, the cooling rate is in the range 500-1000 K/s17, compared to shelf freezing where the cooling rate is on the scale of 1 to 10 K/min. The faster cooling results in smaller ice crystals. Second, the TFFD process creates thin films with thickness below one millimeter, and the free space in the thin films provides channels for water to travel in the sublimation process. It is not known if the gas-liquid interfacial tension between liquid droplets and air during the dropping and freezing process causes aggregation on the nanoparticles, but the gas-liquid interfacial tension is lower than during spray freezing. Finally, the slight increase of the hydrodynamic size of the SLNs after they were subjected to TFFD and reconstitution may not be biologically significant for pulmonary delivery, as the particle size remained smaller than 200 nm and the functionality of the siRNA in the SLNs was not compromised. If needed, future efforts involving in modifying the excipients and the freezing and lyophilization procedures may be applied to minimize particle size change.

Thus, the studies show that thin-film freeze-drying can be applied to prepare dry powder of solid lipid nanoparticles, encapsulated with siRNA or siRNA-free, with good aerosol properties for potential pulmonary delivery to treat lung diseases.

C. The Aerosol Performance of TNF-α siRNA Solid Lipid Nanoparticles for Potential Pulmonary Delivery

1. Methods

The siRNA-solid lipid nanoparticles were engineered by encapsulating TNF-α siRNA complexed with a cationic lipid into solid lipid nanoparticles prepared with lecithin, cholesterol, and a polyethylene glycol (2000)-hydrazone-stearic acid (C18) derivative by nanoprecipitation. The nanoparticles were fluorescently labeled with TopFluor cholesterol. To prepare a dry powder formulation of the siRNA-solid lipid nanoparticles, mannitol was added to the nanoparticle suspension, and the suspension was then freeze-dried. The aerosol performance of the dry powder was examined using a next generation impactor (NGI).

For next generation impactor (NGI) experiment to evaluate the aerosol performance, the nanoparticles were fluorescently labeled with TopFluor cholesterol (Bodipy labelled) at 1.25% w/w of the total cholesterol. For spray drying study, the cationic lipid and siRNA was not added to the formulation. TEM image was taken and macrophage uptake study was performed. Buchi B290 spray dryer was used to prepare the dry powder formulation, using mannitol as the excipient. For freeze drying, a preliminary screening was performed for cryoprotectant and the ideal excipient concentration. The aerosol performance of the SLNs was determined by NGI.

2. Results and Conclusion

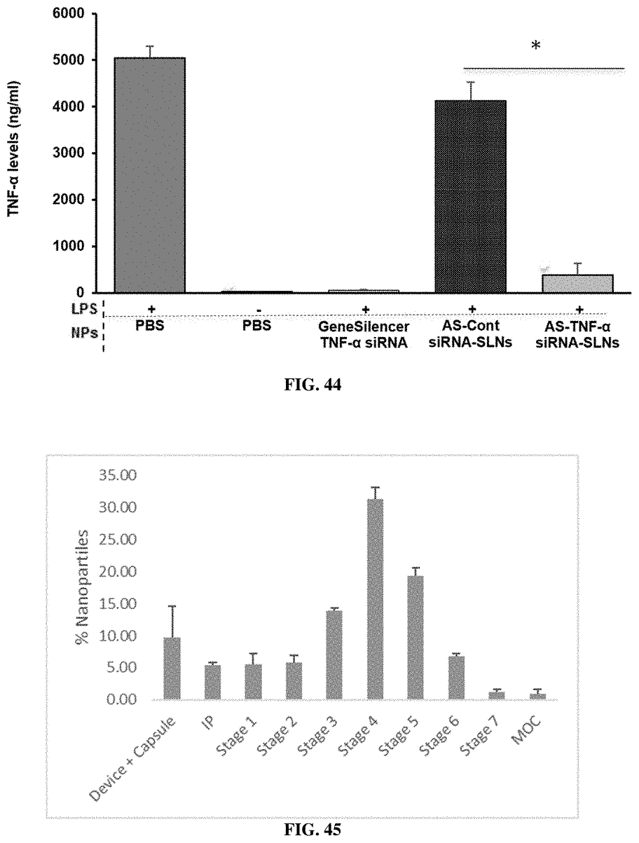

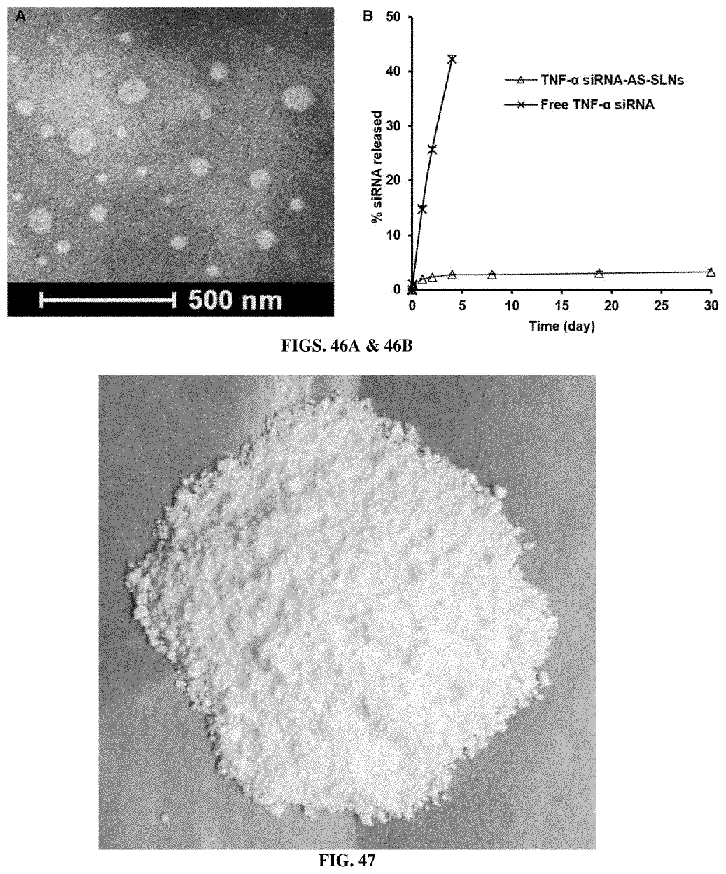

The TNF-α siRNA solid lipid nanoparticles were spherical. Their particle size and polydispersity Index were 118±7 nm and 0.16±0.01. In cell culture, the TNF-α siRNA solid lipid nanoparticles significantly downregulated the expression of TNF-α by J774A.1 mouse macrophages treated with lipopolysaccharide (FIG. 44). The NGI data demonstrated the dry powder of the nanoparticles has good aerosol performance with a fine particle fraction (FPF) of 78.5% (FIG. 45). The TNF-α siRNA solid lipid nanoparticles were spherical. Their particle size and polydispersity Index were 118±7 nm and 0.16±0.01. (FIG. 46).

Dry Powder Formulations of SLN. Physical appearance of dry powder formulations of SLN shown in FIG. 47. The specific surface area of spray dried SLN powder was 0.92±0.11 m²/g whereas the freeze-dried powder was 19.34±2.5 m²/g, both determined by Brunauer-Emmett-Teller (BET).

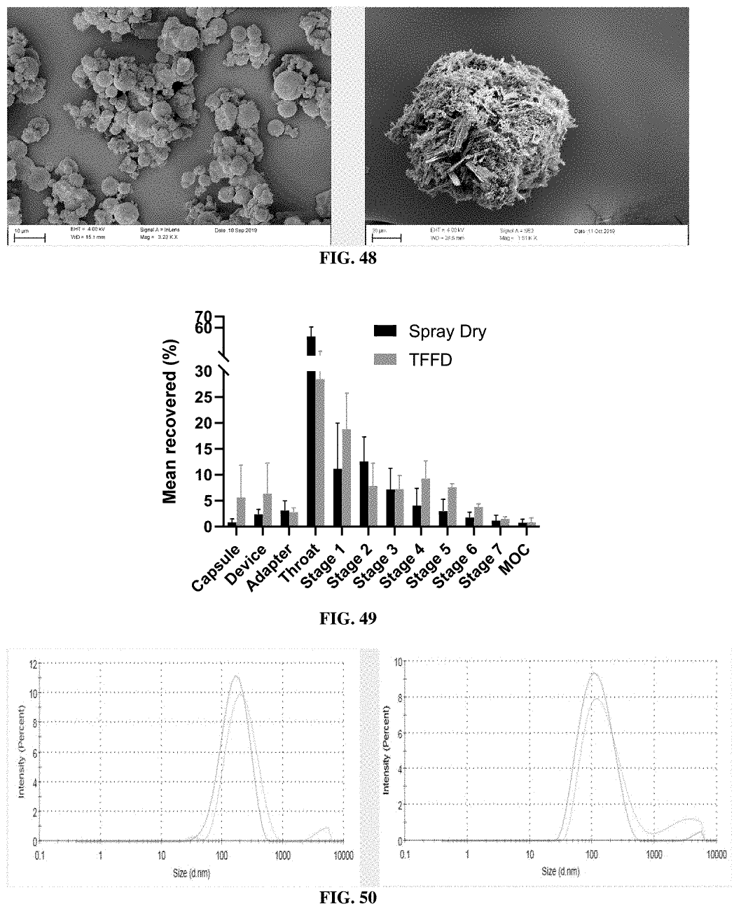

The SEM images showed the freeze-dried powder is more porous than the spray dried powder (FIG. 48). NGI experiment indicated better aerosol performance of freeze-dried powder formulation over the spray dried formulation (FIG. 49). The particle size and PDI increased slightly after both dry methods (Table 16).

TABLE 16
Particle size and PDI before and after each drying method.

| | Particle size difference | PDI difference |
|---|---|---|
| Drying Method | (d · nm) n = 3 | N = 3 |
| Spray drying | +40.17 | +0.121 |
| Freeze drying | +46.5 | +0.084 |

Comparison of size distribution before and after drying shown in FIG. 50. Excipients screening using different buffer and/or cryoprotectants to suspend the nanoparticle before drying steps was also performed, but no effective condition was obtained.

Thus the study shows that TNF-α siRNA solid lipid nanoparticle formulation was able to successfully inhibit TNF-α production by macrophages in culture and alleviated chronic inflammation in mouse model. A dry powder of the nanoparticles showed good aerosol performance for pulmonary delivery.

### Example 5—Thin-Film Freezing and Thin-Film Freeze-Drying of Bacteria

### A. Results

1. Thin-Film Freezing of Bacteria

A single colony of Escherichia coli DH5a (Invitrogen, Carlsbad, Calif.) was inoculated into 3 mL Loria Bertani broth (LB) medium (Invitrogen) starting culture and then transferred to 100 mL LB medium and incubated overnight at 33° C. with shaking. The bacteria were harvested by centrifugation at 2000 rcf for 15 min and washed with cold phosphate-buffered saline (PBS, pH7.4, 10 mM) once. After centrifugation, the bacteria were resuspended to a solution with 10% (w/v) sucrose to the original volume. For thin-film freezing, 250 μL of the bacterial suspension (0.7-5×10⁸ colony forming units (CFU) per ml) was added dropwise using a 21 Gauge needle attached to a syringe to the bottom of a 20 mL glass vial that was pre-cooled with dry ice. The glass vial with the frozen thin-films of bacteria was then capped and placed at room temperature to thaw or stored at −80° C. until further testing. Shelf freezing was used as a control. Briefly, 250 μL of the bacterial suspension was dispensed in a 20 mL glass vial and then frozen at −20° C. for 2 h. The number of live bacteria in the suspension, before or after freeze-and-thawing, was determined using the standard plate assay with LB agar plates following serial dilution with LB medium. Shown in Table 17 are the percent of live bacterial recovered and log CFU reduction after the bacteria were subjected thin-film freezing or shelf freezing. Overall, more bacteria remained alive after they were subjected to thin-film freezing than shelf freezing.

TABLE 17
A comparison of bacterial viability after they were
subjected to shelf freezing or thin-film freezing.

| | | Shelf | Thin-film |
|---|---|---|---|
| | | freezing | freezing |
| Experiment 1 | % recovery | 14.7% | 88.9% |
| | Log CFU reduction | 0.83 | 0.05 |
| Experiment 2 | % recovery | 86.2% | 97.7% |
| | Log CFU reduction | 0.06 | 0.01 |

2. Thin-Film Freeze-Drying of Bacteria

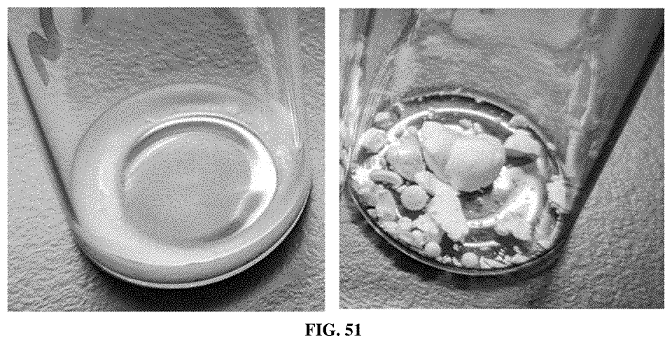

In order to prepare bacterial dry powder, bacteria suspended in 10% sucrose (w/v) was subjected to a standard lyophilization cycle (i.e. sample was dried with a Virtis Advantage freeze dryer (Warminster, Pa.); pressure was <10 mbar; shelf temperature was −40° C. for 24 h, ramped to 25° C. in 24 h, and then hold at 25° C. for 24 h, or Method A in Table 18). The dry powder was then reconstituted with LB medium and the number of live bacteria in the suspension was determined by the plate assay after serial dilution with sterile PBS (pH 7.4, 10 mM). Surprising, only 0.09% of the bacteria were alive, a log reduction of more than 3 (Table 19). Therefore, the effect of the lyophilization cycle as well as the composition of the excipient(s) on the viability of the bacteria after they were subjected to thin-film freeze-drying and reconstitution were studied (Tables 18 and 19). Ultimately, a composition and lyophilization method was found that preserved close to 30% of the bacteria (i.e. log reduction of 0.54) (Table 19). FIG. 51 shows that bacterial dry powder prepared with thin-film freeze-drying is different from that prepared by shelf freeze-drying.

TABLE 18
Lyophilization conditions used to remove water
from thin-film frozen bacterial thin-films.

| Method | Freeze dryer | Lyophilization cycle |
|---|---|---|
| A | Virtis Advantage freeze | Pressure <10 mbar, a shelf temperature −40° C. for |
| | dryer (Warminster, PA) | 24 h, ramped to 25° C. in 24 h, and then hold at 25° C. |
| | | for 24 h |
| B | Labconco manifold | Overnight drying, pressure <0.2 mbar, at ambient |
| | freeze drier | temperature |
| | (Kansas City, MO) | |
| C | Labconco manifold | overnight drying, pressure <0.2 mbar, manifold |
| | freeze drier | placed in ice bath |
| | (Kansas City, MO) | |

TABLE 19
Thin-film freeze-drying of E. coli using different excipients and lyophilization methods (TFF, thin-film freezing;
flash freezing, a 20 mL glass vial with 250 μL of bacterial suspension frozen in liquid nitrogen for less than 1 min).

| | 1 | 2 | 3 | 4 | 5 | 6 | 7 | 8 | 9 |
|---|---|---|---|---|---|---|---|---|---|
| Experiment | Jan. 25, 2020 | Feb. 5, 2020 | Feb. 8, 2020 | Mar. 1, 2020 | Mar. 1, 2020 | Mar. 1, 2020 | Mar. 1, 2020 | Mar. 1, 2020 | Mar. 1, 2020 |
| Date | | | | | | | | | |
| Freezing | TFF | TFF | TFF | Flash | Flash | TFF | TFF | TFF | TFF |
| method | | | | freezing | freezing | | | | |
| Drying | A | B | B | C | C | C | C | C | C |
| Method | | | | | | | | | |
| Excipients | 10% | 10% | No | 10% | 1.7% | 1.7% | Sucrose | 1.7% | 1.7% |
| (w/v) | sucrose | sucrose | | Sucrose | trehalose | trehalose | 5% & | trehalose | trehalose |
| | | | | | | | 3.75% | in 1 mM | & 3.75% |
| | | | | | | | Mannitol | CaCl2 | mannitol |
| % Viability | 0.09% | 1.82% | 1.04% | 17.78% | 0.00% | 1.27% | 10.48% | 0.32% | 28.57% |
| Log CFU | 3.04 | 1.74 | 1.98 | 0.75 | N/A | 1.90 | 0.98 | 2.49 | 0.54 |
| reduction | | | | | | | | | |

3. Freeze and Thaw

Table 20 shows results of freeze and thaw experiments. Cells were centrifuged on 4000 RPM for 30 min and then resuspended in 10% w/v sucrose solution. For the unfreeze group experiment, 100 μL suspension underwent serial dilution directly. For the shelf-freeze experiment, 500 μL of the suspension was placed on −20° C. fridge for 30 min and then warmed to RT. For the TFF experiment, 250 μL of the suspension was dropped to a 20 mL glass vial that was pre-cooled in dry ice-ethanol bath, then warmed to RT directly.

TABLE 20
Results of freeze and that experiments.

| | Plate count | | |
|---|---|---|---|
| Dilution | Unfreeze | Shelf-freeze | TFF |
| 10\hat{ }−5 | 300+ | 106 | 300+ |
| 10\hat{ }−6 | 72 | 10 | 64 |
| 10\hat{ }−7 | 4 | 1 | 8 |
| CFU | 7.20E+07 | 1.06E+07 | 6.40E+07 |
| % Recover | 100.00% | 14.72% | 88.89% |

Beyond the preparation of a thin-film freeze-dried bacterial dry powder, it is also contemplated that the methods disclosed herein may also be applied to prepare dry powder formulations of other organisms such as fungi, yeasts, archaea, viruses, pollens, etc. The organisms may be live, attenuated, or inactivated.

### B. Further Optimization of TFF Bacteria Preparation

In a separate study, bacteria were performed using thin-film freezing on a stainless-steel drum and water was sublimed from the frozen thin-films using a Virtis Advantage Pro lyophilizer (Warminster, Pa.). In brief, a single colony of E. coli DH5a with ampicillin resistant pUC19 vector (Invitrogen, Carlsbad, Calif.) was inoculated into 5 mL Miller Loria Bertani broth (LB) medium (Invitrogen) starting culture overnight and then transferred to 100 mL LB medium and incubated at 37° C. with shaking until OD600 reaches 0.4. The bacteria were harvested by centrifugation at 4300 rcf for 5 min at ambient temperature. After centrifugation, the bacteria were resuspended to cryoprotectant cocktails at 10% of the original culture volume. For thin-film freezing, 1000 μL of the bacterial suspension (0.1-2×10⁹ colony forming units (CFU) per ml) was added dropwise using a 21 Gauge needle attached to a syringe to the rotating stainless drum pre-cooled to −40° C. The frozen films were collected to a 5 mL amber glass vial stored at −80° C. until lyophilization using cycle shown in Table 21. The number of live bacteria in the suspension, before or after subject to the TFFD process, was determined using the standard serial dilution method with LB medium and spread to LB agar plates.

TABLE 21
Lyophilization Cycle for Bacteria using
a Virtis Advantage Pro 85 lyophilizer.

| | | | | |
|---|---|---|---|---|
| | Shelf | Time | | Pressure |
| Step | Temperature | (min) | Ramp/Hold | (mTorr) |

| | | | | | |
|---|---|---|---|---|---|
| 1 | −40° | C. | 60 | H | 100 |
| 2 | −25° | C. | 15 | R | 100 |
| 3 | −25° | C. | 960 | H | 100 |
| 4 | 25° | C. | 50 | R | 100 |
| 5 | 25° | C. | 120 | H | 100 |
| 6 | 4° | C. | 21 | R | 100 |
| End | 4° | C. | Storage | | 390 |

Shown in Table 22 are the different formulations of cryoprotectant cocktails, the CFU count and the log CFU reductions after the bacteria were subjected thin-film freeze-drying. A few formulations can minimize the loss of viability within one log after the bacteria were subjected to thin-film freezing than shelf freezing.

TABLE 22
Cryoprotectant cocktails and bacterial viability after TFFD

| | | CFU/mL | Log |
|---|---|---|---|
| | | after | reduction |
| | | TFFD | after |
| # | Cryoprotectant composition | (n = 1) | TFFD |
| 1 | 0.625% (w/v) LB medium, 10% (w/v) sucrose | 8.00E+07 | 1.4 |
| 2 | 0.3384% (w/v) M9 minimal salt was supplemented with | 5.20E+08 | 0.6 |
| | magnesium sulfate (2 mM) and calcium chloride (0.1 mM), | | |
| | 10% (w/v) sucrose | | |
| 3 | 0.625% (w/v) LB medium, 1.5% (w/v) PVP-40, 10% (w/v) | 3.20E+06 | 2.8 |
| | trehalose | | |
| 4 | 0.625% (w/v) LB medium, 10% (w/v) sucrose, 1% (w/v) | 4.08E+07 | 1.7 |
| | Poloxamer P188 | | |
| 5 | 0.625% (w/v) LB medium, 10% (w/v) sucrose | 2.92E+08 | 0.8 |
| | (resuspended to 4% of the original culture volume) | | |
| 6 | 0.625% (w/v) LB medium, 10% (w/v) sucrose, 0.75 mg/mL | 7.20E+08 | 0.4 |
| | L-leucine | | |
| 7 | 0.625% (w/v) LB medium, 10% (w/v) sucrose, 1.5% (w/v) | 2.62E+07 | 1.9 |
| | PVP-40 | | |

### Example 6—Thin-Film Freezing and Thin-Film Freeze-Drying of Plasmid DNA

### A. Materials and Methods

i. Materials

The β-galactosidase gene-encoding plasmid DNA pCMV-β was from the American Type Culture Collection (ATCC, Manassas, Va.), It was constructed based on pUC19 plasmid capable of expressing E. coli beta-galactosidase (β-Gal) under the control of different viral promoters in mammalian cells (MacGregor et al., 1989). E. coli DH5a competent cells and LB broth were from Invitrogen (Carlsbad, Calif.). The 1,4-dioxane and tert-butanol, Tris-EDTA (TE) buffer, and ampicillin were from Fisher Scientific (Fair Lawn, N.J.). Agarose was from Amresco (Atlanta, Ga.). Polysorbate 20, lactose monohydrate, and methanol anhydrate were from Sigma-Aldrich (St. Louis, Mo.). Quant-iT™ PicoGreen™ dsDNA Assay Kit was from Thermo Scientific (Waltham, Mass.). Size #3 hydroxypropyl methylcellulose capsules were from Quali-V-I capsules (Qualicaps US, Whitsett, N.C.).

ii. Plasmid Preparation

The pCMV-β plasmid was transformed into E. coli DH5a under selective growth conditions and then amplified and purified using a QIAGEN Midiprep Kit (Valencia, Calif.). Large scale plasmid preparation was performed by QIAGEN Plasmid Maxi kit. The plasmid was evaluated using agarose gels and Nanodrop 2000 Spectrophotometers from Thermo Scientific (Waltham, Mass.)

iii. Preparation of Plasmid DNA Dry Powder Using Thin Film Freezing

To screen for the best formulation of dry powder for inhalation, pCMV-β and excipients (i.e., mannitol and leucine) were dissolved in either water, Tris-EDTA (TE) buffer, 1,4-dioxane/water (10/90, v/v), or Tert-butanol/water (40/60, v/v) at various solid contents and plasmid loading as shown in Table 23. The formulations were temporarily stored in a refrigerator at 2-8° C. before applied to the thin-film freezing process.

TABLE 23
List of plasmid compositions and TFF parameters.

| | Plasmid | | | Solid | | Processing |
|---|---|---|---|---|---|---|
| | loading | Excipient ratio (w/w) | | content | | Temperature |
| Formulation | (% w/w) | Mannitol | Leucine | (% w/v) | Solvent | (° C.) |
| P1 | 5 | 7 | 3 | 1 | Water | −80 |
| P2 | 10 | 7 | 3 | 1 | Water | −80 |
| P3 | 5 | 7 | 3 | 0.25 | Water | −80 |
| P4 | 5 | 7 | 3 | 0.5 | Water | −80 |
| P5 | 10 | 7 | 3 | 0.25 | Water | −80 |
| P6 | 2.5 | 7 | 3 | 0.25 | Water | −80 |
| P7 | 5 | 7 | 3 | 0.25 | TE buffer | −80 |
| P8 | 5 | 7 | 3 | 0.25 | 1,4-dioxane/water | −80 |
| P9 | 5 | 7 | 3 | 0.25 | Terf-butanol/water | −80 |

TFF process and lyophilization was done as previously described (Li et al., 2015; Sahakijpijarn et al., 2020a; Moon et al., 2019; Sahakijpijarn et al., 2020b). Briefly, 0.25 mL of sample was dropped through a 21-gauge syringe dropwise onto a rotating cryogenically cooled stainless-steel surface (−80±10° C.). To form frozen thin-films, the speed at which the cryogenically cooled steel surface of the drum rotated was controlled at 5-7 rpm to avoid the overlap of droplets. The frozen thin-films were removed using a steel blade and collected in liquid nitrogen in a glass vial. The glass vial was capped with a rubber stopper with half open and transferred into a −80.0 freezer (Thermo Fisher Scientific) for a temporary storage, and then transferred to a VirTis Advantage bench top tray lyophilizer with stopper re-cap function (The VirTis Company, Inc. Gardiner, N.Y.). Lyophilization was performed over 60 h at pressures no more than 100 mTorr, while the shelf temperature was gradually ramped from −40° C. to 25° C. The lyophilization cycle is shown in Table 24.

TABLE 24
Lyophilization cycle used to lyophilize
the thin-film frozen plasmids.

| Lyophilization Stage | Parameters | |
|---|---|---|
| Loading/Freezing temp | −40° | C. |
| Primary drying temp | −40° | C. |
| Primary drying time | 20 | h |
| Ramp to secondary drying | 20 | h |
| Secondary drying temp | +25° | C. |
| Secondary drying time | 20 | h |

iv. In Vitro Aerosol Performance Evaluation

The aerosol performance properties of the thin-film freeze-dried plasmid powder samples were determined as previously described (Li et al., 2015; Sahakijpijarn et al., 2020a; Moon et al., 2019; Sahakijpijarn et al., 2020b). Briefly, a Next Generation Pharmaceutical Impactor (NGI) (MSP Corp, Shoreview, Minn.) connected to a High-Capacity Pump (model HCPS, Copley Scientific, Nottingham, UK) and a Critical Flow Controller (model TPK 2000, Copley Scientific, Nottingham, UK) was adopted to assess the aerosol performance. To avoid emitted particles bounce across NGI collection plates, the plates were precoated with 1.5%, w/v, polysorbate 20 in methanol and dried in air before analysis. Plasmid DNA powder (2-3 mg) was loaded into a Size #3 capsule, and the capsule was loaded into a high-resistance Plastiape® RS00 inhaler (Plastiape S.p.A, Osnago, Italy) attached to a United States Pharmacopeia (USP) induction port (Copley Scientific, Nottingham, UK). The powder was dispersed to the NGI at the flow rate of 60 L/min for 4 s per each actuation, providing a 4 kPa pressure drop across the device. Then, the deposited powders from the capsule, inhaler, adapter, induction port, stages 1-7, and the micro-orifice collector (MOC) were collected by diluting with water, and the amount of plasmid DNA deposited was quantified using a PicoGreen™ dsDNA Assay Kit following manufacturer's instruction.

The Copley Inhaler Testing Data Analysis Software (CITDAS) Version 3.10 (Copley Scientific, Nottingham, UK) was used to calculate the mass median aerodynamic diameter (MMAD), the geometric standard deviation (GSD), and the fine particle fraction (FPF). The FPF of recovered dose was calculated as the total amount of plasmid collected with an aerodynamic diameter below 5 μm as a percentage of the total amount of plasmid collected. The FPF of delivered dose was calculated as the total amount of plasmids collected with an aerodynamic diameter below 5 μm as a percentage of the total amount plasmids deposited on the adapter, the induction port, stages 1-7 and MOC.

v. Scanning Electron Microscopy (SEM)

The morphology of powder was examined using a Zeiss Supra 40C scanning electron microscope (SEM) (Carl Zeiss, Heidenheim an der Brenz, Germany) in the Institute for Cell and Molecular Biology Microscopy and Imaging Facility at The University of Texas at Austin. A small amount of bulk powder (e.g., a flake of the thin-Film Freeze-Dried powder) was deposited on the specimen stub using a double-stick carbon tape. A sputter was used to coat the sample with 15 mm of 60/40 of Pd/Pt before capturing images.

vi. Agrose Gel Electrophoresis

Plasmid pCMV-β was formulated into Formulation P7 (Table 23) and thin-film freeze-dried. The dry powder was then reconstituted and then digested with EcoR I or Hind III and EcoR I for 2 hours and applied to agarose gel (0.8%) for electrophoresis. Controls include pCVM-β alone or pCMV-β in Formulation P7 without thin-film freeze-drying, both digested and applied to electrophoresis.

### B. Results

Mannitol and leucine at a ratio of 7:3, w/w, were chosen as the excipients for thin-film freeze-drying plasmid DNA. Data showed that placebo powder prepared with mannitol and leucine, 7:3, w/w, at a solid content of 1%, w/v, had excellent aerosol performance properties, with an MMAD value of 0.99±0.25 μm, GSD of 2.39±0.09, recovered FPF of 84.7±9.0%, delivered FPF of 91.1±5.5%, and emitted dose (ED) of 92.7±3.9%.

i. In Vitro Aerosol Performance

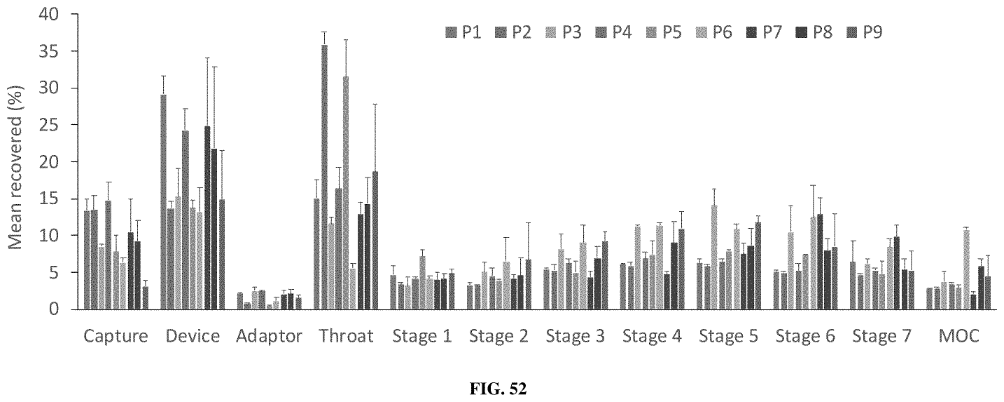

The aerosol performance properties of the thin-film freeze-dried plasmid DNA dry powders are shown in FIG. 52 and Table 25. It is clear that dry powders prepared with lower solid contents showed better aerosol performance. For example, the FPF_{<5 μm} (of the recovered dose) of plasmid formulations prepared with 1.0, 0.5 and 0.25%, w/v, of solid content (P1, P4 and P3) were 32.92±2.52%, 34.55±2.34% and 55.13±2.36%, respectively, and the MMAD values of these powders were 1.58±0.07 μm, 1.77±0.22 μm and 1.44±0.16 μm, respectively (Table 25). As to the effect of the plasmid loading (plasmid vs. total excipients) on the aerosol performance, lower plasmid loading showed better aerosol performance. For example, the FPF_{<5 μm} (of the recovered dose) of plasmid formulations prepared with 10.0, 5.0 and 2.5%, w/w, of plasmid (P5, P3 and P6, respectively) were 36.13±2.53%, 55.13±2.36% and 64.70±3.53%, respectively and the MMAD values of these powders were 1.69±0.30 μm, 1.44±0.16 μm and 1.27±0.40 μm, respectively (Table 25). However, considering the actual amount of delivered dose into deep lung (FPF delivered dose multiplied by drug loading), Formulation P3 (5% plasmid DNA loading, 0.25% solid content) was considered the best formulation.

The effect of co-solvent and TE buffer on the aerosol performance was also investigated. Including TE buffer, 1,4-dioxane, or Tert-butanol in the solvent did not help improve the FPF_{<5 μm} (of the recovered dose) (FIG. 52, Table 25). However, it is noted that the TE buffer in P7 was intended to protect plasmid DNA from DNase. The EDTA in the TE buffer is a chelator of divalent cations such as Mg²⁺, which is required by the enzyme (Nurakami et al., 2013). It appeared that including the TE buffer in the solvent slightly reduced the aerosol performance of the resultant dry powder (P3 vs. P7, in FIG. 52 and Table 25). In the future, if the stability of the plasmid during or after TFFD needs improvement, then TE buffer or ETDA alone may be included in the powder.

TABLE 25
Aerosol performance properties of thin-film freeze-dried
pCMV-β plasmid powders. Data are mean ± S.D.
(n = 3) (MMAD, mass median aerodynamic diameter;
GSD, geometric standard deviation; FPF, fine particle fraction).

| | | | FPF | FPF |
|---|---|---|---|---|
| | | | (recovered) | (delivered) |
| Formulation | MMAD | GSD | % | % |
| P1 | 1.58 ± 0.07 | 3.73 ± 0.67 | 32.92 ± 2.52 | 57.26 ± 3.19 |
| P2 | 1.62 ± 0.10 | 3.19 ± 0.29 | 30.27 ± 1.12 | 41.58 ± 1.80 |
| P3 | 1.44 ± 0.16 | 2.77 ± 0.19 | 55.13 ± 2.36 | 72.32 ± 0.41 |
| P4 | 1.77 ± 0.22 | 3.15 ± 0.21 | 34.55 ± 2.34 | 56.63 ± 3.82 |
| P5 | 1.69 ± 0.30 | 4.24 ± 1.38 | 36.13 ± 2.53 | 46.16 ± 4.44 |
| P6 | 1.27 ± 0.40 | 4.24 ± 1.38 | 64.70 ± 3.53 | 80.39 ± 3.23 |
| P7 | 0.96 ± 0.05 | 3.30 ± 0.67 | 42.45 ± 4.73 | 65.68 ± 4.12 |
| P8 | 1.50 ± 0.15 | 3.11 ± 0.54 | 44.94 ± 7.27 | 65.01 ± 4.22 |
| P9 | 1.74 ± 0.19 | 2.85 ± 1.20 | 51.92 ± 10.52 | 62.96 ± 8.86 |

ii. Morphology of the thin film freeze-dried plasmid DNA powder

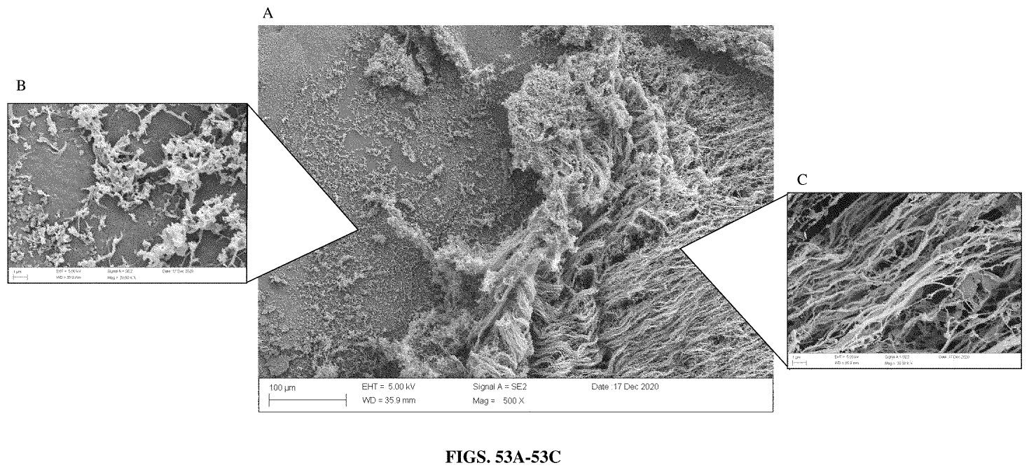

The morphology of the plasmid powder prepared by TFFD (formulation P3) was examined using SEM (FIG. 53). Dry powder formulation P3 contained nanostructure aggregates (FIGS. 53A & 53B), with highly porous matrix structure (FIG. 53C), which explains the good aerosol performance properties as shown in FIG. 52 and Table 25.

iii. Integrity of Plasmid DNA after Subject to TFFD

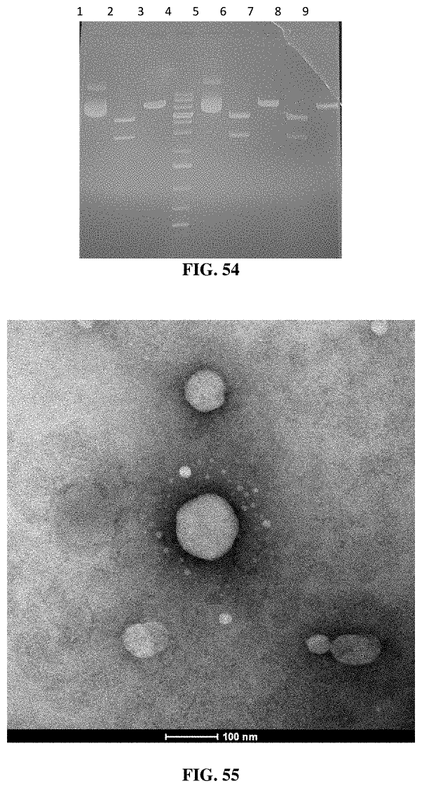

Formulation 7 has 5% plasmid DNA loading, contains TE, and have overall good aerosol performance properties. This formulation was chosen to test the integrity of the plasmid DNA after it was subjected to TFFD and reconstitution. Plasmid pCMV-β was formulated to Formulation 7 and thin-film freeze-dried. It was then reconstituted, digested with EcoR I or Hind III and EcoR I for 2 hours and applied to agrose gel for electrophoresis. Controls include pCVM-β alone or pCMV-β in Formulation 7 without thin-film freeze-drying, digested and applied to electrophoresis. As shown in FIG. 54, subjecting pCMV-β to TFFD did not cause any significant change in the plasmid integrity.

Taken together, it is concluded that thin-film freeze-drying can be applied to transform pure plasmid DNA into aerosolizable dry powders while preserving it chemical integrity.

### Example 6—Thin-Film Freezing and Thin-Film Freeze-Drying of mRNA-LNPs

### A. Preparation of TFF-mRNA/LNP Dry Powder

Formulation 1: To a scintillation vial, 3.5 mL of poloxamer 188 (1.0 mg/mL) was added, followed by the addition 10.0 mL of a mRNA COVID-19 vaccine that has received emergency use authorization (diluted, 2.567 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer. The solvent was removed by lyophilizer by a processing of holding at −40° C. for 20h at or below 100 mTorr, ramping to 25° C. for 20h at 100 mTorr, and holding at 25° C. for 5h at 100 mTorr. The dry nitrogen gas was backfilled, and the lid of the vial was closed by the stoppering system before open the lyophilizer door. The vial was sealed with an aluminum cap for storage.

Formulation 2: To a scintillation vial, 10.5 mL of sucrose (20.0 mg/mL) and 4.2 mL of poloxamer 188 (1.0 mg/mL) were added, followed by the addition of 3.0 mL of a mRNA COVID-19 vaccine (diluted, 2.567 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer. The solvent was removed by lyophilizer by a processing of holding at −40° C. for 20h at or below 100 mTorr, ramping to 25° C. for 20h at 100 mTorr, and holding at 25° C. for 5h at 100 mTorr. The dry nitrogen gas was backfilled, and the lid of the vial was closed by the stoppering system before open the lyophilizer door. The vial was sealed with an aluminum cap for storage.

Formulation 3: To a scintillation vial, 8.0 mL of trehalose (20.0 mg/mL) and 4.6 mL of poloxamer 188 (1.0 mg/mL) were added, followed by the addition of 2.0 mL of a mRNA COVID-19 vaccine (diluted and dialyzed to remove excipients, 2.127 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer. The solvent was removed by lyophilizer by a processing of holding at −40° C. for 20h at or below 100 mTorr, ramping to 25° C. for 20h at 100 mTorr, and holding at 25° C. for 5h at 100 mTorr. The dry nitrogen gas was backfilled, and the lid of the vial was closed by the stoppering system before open the lyophilizer door. The vial was sealed with an aluminum cap for storage.

Formulation 4: To a scintillation vial, 8.0 mL of sucrose (20.0 mg/mL) and 4.6 mL of poloxamer 188 (1.0 mg/mL) were added, followed by the addition of 2.0 mL of a mRNA COVID-19 vaccine (diluted and dialyzed to remove excipients, 2.127 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer. The solvent was removed by lyophilizer by a processing of holding at −40° C. for 20h at or below 100 mTorr, ramping to 25° C. for 20h at 100 mTorr, and holding at 25° C. for 5h at 100 mTorr. The dry nitrogen gas was backfilled, and the lid of the vial was closed by the stoppering system before open the lyophilizer door. The vial was sealed with an aluminum cap for storage.

Formulation 5: To a 200 μL centrifuge tube, 40 μL of sucrose (20.0 mg/mL) and 13 μL of poloxamer 188 (1.0 mg/mL) were added, followed by the addition of 10 μL of a mRNA COVID-19 vaccine (diluted and dialyzed to remove excipients, 2.16 mg LNP/mL). The mixture was gently shaken and dropped dropwise onto the cryogenically cooled (−180° C.) stainless steel drum. The frozen sample was collected in a stainless-steel container, filled with liquid nitrogen. The sample was transferred in a glass lyophilized vial and stored in a −80° C. freezer until placing in a lyophilizer. The solvent was removed by lyophilizer by a processing of holding at −40° C. for 20h at or below 100 mTorr, ramping to 25° C. for 20h at 100 mTorr, and holding at 25° C. for 5h at 100 mTorr. The dry nitrogen gas was backfilled, and the lid of the vial was closed by the stoppering system before open the lyophilizer door. The vial was sealed with an aluminum cap for storage.

Shelf freeze-drying: For mRNA-LNP formulations 1, 2, and the original mRNA COVID vaccine upon dilution as mentioned above, dry powders were also prepared with conventional shelf freeze-drying. The mRNA-LNPs in suspension (0.6 mL) were placed into 2 mL lyophilized vials and the vials were placed in an Advantage EL shelf freeze dryer. The shelf temperature was cooled from room temperature to −50° C. at the rate of 1° C./min and maintained at 50° C. for 1 h before drying. The drying cycle was the same as one used to sublime water from the thin-film frozen samples.

### B. Dialysis

The approved mRNA COVID vaccines were dialyzed against at least 1,000 fold-volume of diethyl pyrocarbonate (DEPC)-treated water at 4° C. for 24 h. The concentration of LNPs was then adjusted based on the volume change after dialysis.

For example, 1.200 mL of the approved mRNA COVID vaccine was placed into a dialysis tube (Spectrum, Stamford, Conn.), then the dialysis tube was placed in 1,500 mL of DEPC-treated water in an external beaker with a gentle stirring speed of 100 rpm at 4° C. for 24 h. The dialysis solution (DEPC-treated water) was changed every 8 h. Finally, 1.398 mL of sample was recovered from the dialysis tube. The concentration of LNPs was calculated based on the volume change for the formulation preparation for TFF.

### C. Characterization of the TFF-mRNA/LNP Dry Powder

i. Particle Size Distribution (PSD)

A small quantity of TFF powder was placed into a disposable UV cuvette and reconstituted with filtered water (Evoqua, Warrendale, Pa.). Particle size distribution was measured using a Zetasizer Nano ZS (Malvern Panalytical Ltd, Malvern, UK) with dispersant refractive index of 1.33 and material refractive index of 1.45. Shown in Table 1 below are the particle size (Z-average) of the mRNA-LNPs before they were subjected to thin-film freeze-drying (TFFD), after they were subjected to TFFD and reconstitution, and after the dry powders were storated at in a refrigerator (−4° C.) or at temperature (−25° C.) for three weeks.

TABLE 26
Particle size distribution of dry powder. Data are mean ± SD (n = 3).

| | | | Z-average (d · nm) | | |
|---|---|---|---|---|---|
| | Composition | Freezing | | 25° C., | 4° C., |
| Formulation | (weight ratio)* | Method | Initial | 3 weeks | 3 weeks |
| mRNA/LNP | 8.5 LNPs/ | Liquid | 87.8 ± 2.2 | — | — |
| COVID Vac | 63.8 Sucrose/ | | | | |
| | 27.7 PBS, | | | | |
| | diluted in | | | | |
| | normal saline | | | | |
| | | Shelf-freeze | ** | — | — |
| mRNA/LNP | 100 LNPs | Liquid | 95.2 ± 0.4 | — | — |
| COVID Vac, | with sucrose, | | | | |
| Dialyzed | buffer and | | | | |
| | salts | | | | |
| | removed | | | | |
| Formulation 1 | 8.4 LNPs/ | TFF | 122.9 ± 3.5 | 144.0 ± 2.2 | 117.9 ± 1.7 |
| | 63.1 Sucrose/ | | | | |
| | 27.4 PBS/ | | | | |
| | 1.1 P188 | | | | |
| | | Shelf-freeze | ** | — | — |
| Formulation 2 | 2.6 LNPs/ | TFF | 151.7 ± 3.6 | 172.7 ± 3.3 | 164.1 ± 5.8 |
| | 87.6 Sucrose/ | | | | |
| | 8.5 PBS/ | | | | |
| | 1.4 P188 | | | | |
| | | Shelf-freeze | ** | — | — |
| Formulation 3 | 2.6 LNPs/ | TFF | 112.0 ± 1.1 | 144.4 ± 1.9 | 115.4 ± 0.9 |
| | 94.7 Trehalose/ | | | | |
| | 2.7 P188 | | | | |
| Formulation 4 | 2.6 LNPs/ | TFF | 116.9 ± 2.0 | 244.3 ± 3.3 | 125.5 ± 1.3 |
| | 94.7 Sucrose/ | | | | |
| | 2.7 P188 | | | | |
| Formulation 5 | 2.7 LNPs/ | TFF | 118.8 | — | — |
| | 95.8 Sucrose/ | | | | |
| | 1.6 P188 | | | | |

*26.67 parts of LNPs includes 1 part of mRNA (w/w)
** Powder did not completely disperse in the reconstitution medium, with large particles floating on the surface of the dispersion medium.

ii. Quantification of mRNA Encapsulation Efficiency

The mRNA loading in a mRNA/LNP COVID vaccine formulation was quantified using a Quanti-iT RiboGreen assay kit (Invitrogen, Carlsbad, Calif.) as previously described (Blakney et al., 2019; Yang et al., 2020). Powder samples were reconstituted to the same concentration as the liquid formulations before TFF process. All samples were diluted two, twenty, two-hundred, and two-thousand times in 1× TE buffer (RNase-free) containing 0.5% (v/v) Triton X-100 (Sigma Aldrich, St. Louis, Mo.) for a 15 min of incubation to detect total mRNA. For detecting free mRNA, all samples were diluted two, twenty, two-hundred, and two-thousand time in 1× TE buffer (RNase-free). All the Triton X-100 treated and untreated samples were incubated with RiboGreen reagent in a black, 96 well-plate (Costar, Corning, N.Y.). The fluorescence intensity was recorded by a BioTek Synergy HT Multi-Mode Microplate Reader (Winooski, Vt., Ex=485 nm, Em=528 nm, gain=35). Fluorscence intensity values were converted to mRNA concentrations based on standard curves built for total mRNA and mRNA outside of LNPs, respectively. The encapsulation efficiency was calculated according to the following formula:

$$Encapsulation efficiency ( EE , \% ) = \frac{total mRNA - free mRNA}{total mRNA} \times 100 \%$$

TABLE 2
Encapsulation efficiency

| | | Encapsulation |
|---|---|---|
| Formulation | | (%) |
| mRNA/LNP COVID Vac | Diluted, original Vac | 87.9 |
| Formulation 1 | Before TFF | 91.5 |
| | After TFF, dried and | 93.0 |
| | reconstituted | |

iii. Transmission Electron Microscope (TEM) Analysis

The morphology of LNP formulations was studied using FEI Tecnai transmission electron microscopy. Thin-film freeze-dried mRNA/LNP powder was reconstituted in water and diluted with purified water to obtain an LNP concentration of 0.1-0.3 mg/mL. Five μL of LNP dispersion was added on a 200-mesh carbon film, copper grid (Electron Microscopy Sciences, Hatfield, Pa.). After one minute, a filter paper was used to gently remove the liquid from the edge of the grid. Five μL of 1% phosphotungstic acid was dropped on the grid to negatively stain the sample. After one minute, a filter paper was used to remove the stain from the edge of the grid. The sample was air-dried before capturing images. See FIG. 55.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.
Abdelwahed et al., Adv. Drug Deliv. Rev., 58(15):1688-1713, 2006.
Akinc et al., Mol. Ther. 17:872-879, 2009.
Aldayel et al., J. Control. Release, 283:280-289, 2018.
Belliveau et al., Mol. Ther. Nucleic Acids, 1:e37, 2012.
Blakney et al., Gene Therapy, 26(9):363-372, 2019.
Chung et al., Int. J. Pharm., 437(1-2):42-50, 2012.
D'Addio et al., Pharm. Res., 30(11):2891-2901, 2013.
Desai and Vadgama, Analyst, 116(11):1113, 1991.
Deshpande et al., AAPS PharmSci., 4(3):12-21, 2002.
Elhissi et al., Int. J. Pharm., 444:193-199, 2013.
Engstrom et al., Pharm. Res., 25(6):1334-1346, 2008.
Huckaby and Lai, Adv. Drug Deliv. Rev., 124:125-139, 2018.
Hyde et al., Hum. Gene Ther. Clin. Dev., 25(2):97-107, 2014.
Jayaraman et al., Angew. Chem. Int. Ed., 51:8529-8533, 2012.
Kauffman et al., Nano Lett., 15:7300-7306, 2015.
Kontogiannopoulos et al., J. Liposome Res., 24:230-240, 2014.
Lball et al., Int. J. Nanomed., 12:305-315, 2017.
Leal et al., Int. J. Pharm., 553(1-2):57-64, 2018.
Leung et al., J. Phys. Chem. B, 119:8698-8706, 2015.
Li et al., Nano Lett., 15:8099-8107, 2015.
Li et al., J. Control Release, 204:38-50, 2015.
Lintingre et al., Soft Matter, 12(36):7435-7444, 2016.
MacGregor et al., Nucleic Acids Research, 17(6):2365, 1989.
Mishra et al., Eur. J. Cell Biol., 83:97-111, 2004.
Moon et al., J. Drug Delivery Science and Technology, 54:101295, 2019.
Murakami et al., The Open Biotechnology J., 7(1), 2013.
Nemati et al., AAPS PharmSciTech, 20(3):1-9, 2019.
Niu and Panyam, J. Control. Release, 248. 125-132, 2017.
Norris and Sinko. J. Appl. Polym. Sci., 63(11): 1481-1492. 1997.
Ohashi et al., J. Control. Release. 135(1): 19-24. 2009.
Osman et al., J. Controlled Rei, 285:35-45. 2018.
Olsuka et al., Adv. Drug Deliv. Rev., 55:403-419. 2003.
Overhoff et al., Eur. J. Pharin. Biopharm., 65(l):57-67, 2007.
Overhoff et al.. Phartn. Res., 25(1): 167-175, 2008.
Patil-Gadhe and Pokharkar, Int. J. Pharm., 501 (1-2): 199-210, 2016.
Pallolla et al.; J. Control. Release. 144(2):233-241. 2010.
Rahimpour and Hamishehkar. Adv. Pharm. Bull., 2(2): 183-187. 2012.
Ran et al., Nat. Protoc., 8(11):2281-2308, 2013.
Sahakijpijarn et al., International J. Pharmaceutics, 586:119490, 2020.
Sahakijpijarn et al., Pharmaceutics, 12(11): 1002. 2020.
Sriwongsitanont and Ueno, Colloid Polymer Sci., 282:753-760, 2004.
Thakkar et al., Hum. Vaccines Immunother., 13(4):936-946, 2017.
Wagner et al., J. Liposome Res., 16:113-125, 2006.
Wang et al., J. Microencapsul., 35(3):241-248. 2018.
Yang ct al., Bioactive Materials. 5(4): 1053-1061, 2020.
Zhang et al., Eur. J. Pharm. Biopharm., 82(3):534-544, 2012.
Zhang et al., Mol. Pharm., 15(11): 4814-4826. 2018.
Zhu et al., Mol. Pharm., 10(9):3525-3530, 2013.

1. A dry powder comprising a biologically active polynucleotides and at least a first excipient, said dry powder having been produced by an ultra-rapid freezing process (URF), wherein the biologically active polynucleotides retain substantial biological activity and/or have been stabilized by the URF process.

2. The dry powder of claim 1, wherein the biologically active polynucleotides retain at least about 0.5% of a biological activity compared to an equal amount of the biologically active polynucleotides in solution prior to the URF process.

3. The dry powder of claim 1, wherein the biologically active polynucleotides have been stabilized such that at least 50% more of the biologically active polynucleotides in the dry powder are undegraded relative the same biologically active polynucleotides in a solution.

4. The dry powder of claim 1, wherein the URF process comprises thin film freezing (TFF).

5.-6. (canceled)

7. The dry powder of claim 1, wherein the biologically active polynucleotides comprise siRNA, shRNA, dsRNA, ssRNA, mRNA, plasmid DNA and/or DNA oligonucleotides.

8. The dry powder of claim 1, wherein the dry powder has a geometric particle size distribution Dv50, measured by dry Rodos method, of less than about 100 μm.

9.-11. (canceled)

12. The dry powder of claim 1, wherein the first excipient comprises a sugar or sugar alcohol.

13.-14. (canceled)

15. The dry powder of claim 1, wherein the first excipient comprises at least about 50% of the dry powder by weight.

16.-21. (canceled)

22. The dry powder of claim 1, further comprising at least a second, third and/or fourth excipient.

23. The dry powder of claim 22, wherein the second, third and/or fourth excipient comprises an amino acid, protein, a polymer, a sugar, a sugar alcohol, or a surfactant.

24.-31. (canceled)

32. The dry powder of claim 1, wherein the biologically active polynucleotides comprises a virus.

33.-48. (canceled)

49. The dry powder of claim 1, wherein the biologically active polynucleotides comprise the biologically active polynucleotides encapsulated in a lipid nanoparticles (LNPs).

50.-92. (canceled)

93. The dry powder of claim 1, wherein the biologically active polynucleotides comprise genomic material.

94. (canceled)

95. The dry powder of claim 1, wherein the dry powder comprises intact bacterial cells.

96.-106. (canceled)

107. An inhaler comprising the dry powder of claim 1.

108.-113. (canceled)

114. A method of producing powder pharmaceutical composition comprising:
(a) admixing a biologically active polynucleotide molecule and a first excipient in a solvent to form a precursor solution;
(b) depositing the precursor solution onto a surface at a temperature suitable to cause the solvent to freeze; and
(c) removing the solvent to obtain the powder pharmaceutical composition.

115. The method of claim 114, further comprising:
(d) disaggregating the powder pharmaceutical composition to reduce particle size and/or homogenize particle size.

116.-118. (canceled)

119. The method of claim 114, wherein the temperature in step (b) is about −40° C. to −180° C.

120.-174. (canceled)

175. A pharmaceutical composition prepared according to the methods of claim 114.

176.-184. (canceled)

185. A method of treating a disease in a subject comprising administering an effective amount of a composition of claim 1 to the subject.

186.-189. (canceled)
